(19) United States
(12) Patent Application Publication
(10) Pub. No.: US 2024/0305768 A1
(43) Pub. Date: Sep. 12, 2024

(54) LIGHT FIELD DISPLAY FOR RENDERING PERCEPTION-ADJUSTED CONTENT, AND DYNAMIC LIGHT FIELD SHAPING SYSTEM AND LAYER THEREFOR

(71) Applicant: EVOLUTION OPTIKS LIMITED, Christ Church (BB)

(72) Inventors: Raul MIHALI, Westport, CT (US); Faleh Mohammad Faleh ALTAL, Montreal (CA); Thanh Quang TAT, Montreal (CA); Mostafa DARVISHI, Brossard (CA); Joseph Ivar ETIGSON, Toronto (CA)

(21) Appl. No.: 18/006,448

(22) PCT Filed: Jul. 23, 2021
(86) PCT No.: PCT/US2021/070944

§ 371 Date: Jan. 23, 2023

Related U.S. Application Data
(60) Provisional application No. 63/056,188, filed on Jul. 24, 2020.
(60) Provisional application No. 63/104,468, filed on Oct. 22, 2020.

(51) Int. Cl. H04N 13/32; H04N 13/305; H04N 13/327; H04N 13/383; H04N 13/398

(52) U.S. Cl. CPC H04N 13/32; H04N 13/305; H04N 13/327; H04N 13/383; H04N 13/398

(57) ABSTRACT

Described are various embodiments of a light field shaping systems for interfacing with light emanated from pixels of a digital display to govern display of perception-adjusted content. Various embodiments comprise a light field shaping layer (LFSL) disposable relative to the digital display to align an array of light field shaping elements with the pixels of the digital display, thereby defining a perception adjustment of displayed content. Various embodiments further comprise an actuator operable on by a digital processor for translating the LFSL to adjust an optical path length between the LSFL and the digital display, thereby defining an adjusted perception adjustment of displayed content in accordance with an adjusted geometry.

### CROSS REFERENCE TO RELATED APPLICATIONS

This application claims priority to U.S. Provisional Application No. 63/056,188 filed Jul. 24, 2020, and to U.S. Provisional Application No. 63/104,468 filed Oct. 22, 2020, the entire disclosure of each of which is hereby incorporated herein by reference.

### FIELD OF THE DISCLOSURE

The present disclosure relates to light field displays, and, in particular, to a light field display for rendering perception-adjusted content, and dynamic light field shaping system and method, and layer therefor.

### BACKGROUND

Individuals routinely wear corrective lenses to accommodate for reduced vision acuity in consuming images and/or information rendered, for example, on digital displays provided, for example, in day-to-day electronic devices such as smartphones, smart watches, electronic readers, tablets, laptop computers and the like, but also provided as part of vehicular dashboard displays and entertainment systems, to name a few examples. The use of bifocals or progressive corrective lenses is also commonplace for individuals suffering from near and farsightedness.

The operating systems of current electronic devices having graphical displays offer certain “Accessibility” features built into the software of the device to attempt to provide users with reduced vision the ability to read and view content on the electronic device. Specifically, current accessibility options include the ability to invert images, increase the image size, adjust brightness and contrast settings, bold text, view the device display only in grey, and for those with legal blindness, the use of speech technology. These techniques focus on the limited ability of software to manipulate display images through conventional image manipulation, with limited success.

The use of 4D light field displays with lenslet arrays or parallax barriers to correct visual aberrations have since been proposed by Pamplona et al. (PAMPLONA, V., OLIVEIRA, M., ALIAGA, D., AND RASKAR, R.2012. “Tailored displays to compensate for visual aberrations.” ACM Trans. Graph. (SIGGRAPH) 31.). Unfortunately, conventional light field displays as used by Pamplona et al. are subject to a spatio-angular resolution trade-off; that is, an increased angular resolution decreases the spatial resolution. Hence, the viewer sees a sharp image but at the expense of a significantly lower resolution than that of the screen. To mitigate this effect, Huang et al. (see, HUANG, F.-C., AND BARSKY, B. 2011. A framework for aberration compensated displays. Tech. Rep. UCB/EECS-2011-162, University of California, Berkeley, December; and HUANG, F.-C., LANMAN, D., BARSKY, B. A., AND RASKAR, R. 2012. Correcting for optical aberrations using multilayer displays. ACM Trans. Graph. (SIGGRAPH Asia) 31, 6, 185:1-185:12) proposed the use of multilayer display designs together with prefiltering. The combination of prefiltering and these particular optical setups, however, significantly reduces the contrast of the resulting image.

Finally, in U.S. Patent Application Publication No. 2016/0042501 and Fu-Chung Huang, Gordon Wetzstein, Brian A. Barsky, and Ramesh Raskar. “Eyeglasses-free Display: Towards Correcting Visual Aberrations with Computational Light Field Displays”. ACM Transaction on Graphics, 33(4), August 2014, the entire contents of each of which are hereby incorporated herein by reference, the combination of viewer-adaptive pre-filtering with off-the-shelf parallax barriers has been proposed to increase contrast and resolution, at the expense however, of computation time and power.

Optical devices, such as refractors and phoropters, are commonly used to test or evaluate the visual acuity of its users, for example, in the prescription of corrective eyewear, contact lenses or intraocular implants.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for a light field display for rendering perception-adjusted content, and dynamic light field shaping system and method, and layer therefor that overcome some of the drawbacks of known techniques, or at least, provides a useful alternative thereto. Some aspects of this disclosure provide examples of such systems and methods.

In accordance with one aspect, there is provided a light field shaping system for interfacing with light emanated from pixels of a digital display to govern display of perception-adjusted content, the system comprising: a light field shaping layer (LFSL) comprising an array of light field shaping elements and disposable relative to the digital display so to align said array of light field shaping elements with the pixels in accordance with a current light field shaping geometry to thereby define a perception adjustment of displayed content in accordance with said current geometry; an actuator operable to adjust an optical path length between said LSFL and the digital display to adjust alignment of said light field shaping elements with the pixels in accordance with an adjusted geometry thereby defining an adjusted perception adjustment of displayed content in accordance with said adjusted geometry; and a digital data processor operable to activate said actuator to translate said LFSL to adjust the perception-adjusted content.

In one embodiment, the LFSL comprises a microlens array.

In one embodiment, the adjusted perception adjustment corresponds to a reduced visual acuity of a user.

In one embodiment, the actuator is operable to translate said LFSL in a direction perpendicular to the digital display.

In one embodiment, the light field shaping geometry relates to a physical distance between the digital display and said LFSL.

In one embodiment, the adjusted geometry corresponds to a selectable range of perception adjustments of displayed content, wherein distinctly selectable geometries correspond with distinct selectable ranges of perception adjustments.

In one embodiment, the distinct selectable ranges comprise distinct dioptric correction ranges.

In one embodiment, the digital data processor is further operable to: receive as input a requested perception adjustment as said adjusted perception adjustment; based at least in part on said requested perception adjustment, calculate an optimal optical path length to thereby define an optimal geometry as said adjusted geometry; and activate said actuator to adjust said optical path length to said optimal optical path length and thereby optimally achieve said requested perception adjustment.

In one embodiment, the digital data processor is further operable to: receive as input feedback data related to a quality of said adjusted perception adjustment; and dynamically adjust said optical path length via said actuator in response to said feedback data.

In one embodiment, the light field shaping system comprises a system for administering a vision-based test.

In one embodiment, the vision-based test comprises a visual acuity examination, and the perception-adjusted content comprises an optotype.

In one embodiment, the vision-based test comprises a cognitive impairment test.

In one embodiment, the actuator selectively introduces an optical path length increasing medium within said optical path length to selectively adjust said optical path length.

In one embodiment, the digital data processor is operable to translate said LFSL to adjust the perception-adjusted content while satisfying a visual content quality parameter.

In one embodiment, the visual content quality parameter comprises one or more of a perception-adjusted content resolution, a corneal beam size, a view zone size, or a distance between a pupil and a view zone edge.

In accordance with another aspect, there is provided a method for dynamically adjusting a perception adjustment of displayed content in a light field display system comprising a digital processor and a digital display defined by an array of pixels and a light field shaping layer (LFSL) disposed relative thereto, the method comprising: accessing display geometry data related to one or more of the light field display system and a user thereof, said display geometry data at least in part defining the perception adjustment of displayed content; digitally identifying a preferred display geometry based, at least in part, on said display geometry data, said preferred display geometry comprising a desirable optical path length between said LFSL and the pixels to optimally produce a requested perception adjustment of displayed content; automatically adjusting said optical path length, via the digital processor and an actuator operable to adjust said optical path length and thereby adjust the perception adjustment of displayed content to said requested perception adjustment.

In accordance with another aspect, there is provided a light field shaping system for interfacing with light emanated from underlying pixels of a digital screen in a light field display to display content in accordance with a designated perception adjustment, the system comprising: a light field shaping layer (LFSL) comprising an array of light field shaping elements and disposable relative to the digital screen so to define a system configuration, said system configuration at least in part defining a subset of perception adjustments displayable by the light field display; an actuator operable to adjust a relative distance between said LFSL and the digital screen to adjust said system configuration; and a digital data processor operable to activate said actuator to selectively adjust said relative distance and thereby provide a preferred system configuration defining a preferred subset of perception adjustments; wherein said preferred subset of perception adjustments comprises the designated perception adjustment.

In one embodiment, the digital data processor is further operable to: receive as input data related to said designated perception adjustment; based at least in part on said data related to said designated perception adjustment, calculate said preferred system configuration.

In one embodiment, the digital data processor is further operable to dynamically adjust said system configuration during use of the light field display.

In accordance with another aspect, there is provided a light field display system for displaying content in accordance with a designated perception adjustment, the system comprising: a digital display screen comprising an array of pixels; a light field shaping layer (LFSL) comprising an array of light field shaping elements shaping a light field emanating from said array of pixels and disposable relative thereto in accordance with a system configuration at least in part defining a subset of displayable perception adjustments; an actuator operable to translate said LFSL relative to said array of pixels to adjust said system configuration; and a digital data processor operable to activate said actuator to translate said LFSL and thereby provide a preferred system configuration defining a preferred subset of perception adjustments; wherein said preferred subset of perception adjustments comprises the designated perception adjustment.

In accordance with another aspect, there is provided a light field shaping layer (LFSL) to be used in conjunction with a digital display comprising an array of digital pixels, wherein an optimal rendering of a perceptively adjusted image is provided by minimizing a spread of light from the display pixels through the LFSL in accordance with the following expression:

$${Δθ}_{Req}^{P} = \frac{Max ( \vert {y}_{p} \vert ) + \frac{{w}_{ppl}}{2}}{{D}_{PL} + {D}_{LE}} + \frac{{W}_{L}}{2 {D}_{PL}} .$$

In accordance with another aspect, there is provided a light field shaping system for performing a vision-based assessment using perception-adjusted content, the system comprising: a pixelated digital display; a light field shaping layer (LFSL) comprising an array of light field shaping elements and disposable relative to the pixelated digital display so to align said array of light field shaping elements with pixels of the pixelated digital display in accordance with a current light field shaping geometry to thereby define a perception adjustment of displayed content in accordance with said current geometry; an actuator operable to adjust an optical path length between said LSFL and the pixelated digital display to adjust alignment of said light field shaping elements with the pixels in accordance with an adjusted geometry thereby defining an adjusted perception adjustment of displayed content in accordance with said adjusted geometry; and a digital data processor operable to activate said actuator to translate said LFSL to adjust the perception-adjusted content for the vision-based assessment.

In one embodiment, the vision-based assessment comprises a cognitive impairment assessment.

In one embodiment, the vision-based assessment comprises a visual acuity assessment.

In one embodiment, the digital data processor is operable to translate said LFSL to adjust the perception-adjusted content while maintaining a visual content quality parameter associated with the vision-based assessment.

In one embodiment, the visual content quality parameter comprises one or more of a perception-adjusted content resolution, a corneal beam size of said perception-adjusted content, a view zone size, or a distance between a pupil and a view zone edge.

In one embodiment, the adjusted perception adjustment comprises a range of perception adjustments corresponding to said adjusted geometry.

In one embodiment, the vision-based assessment comprises the display of content in accordance with an assessment range of perception adjustments.

In one embodiment, the range of perception adjustments corresponds at least in part to said assessment range of perception adjustments.

In one embodiment, the system further comprises an optical component intersecting an optical path of the perception-adjusted content and configured to adjust an optical power of the perception-adjusted content for the vision-based assessment.

In one embodiment, the optical component comprises a lens or a tunable lens.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIGS. 1A and 1B are schematic diagrams of an exemplary light field vision testing or previewing system, in accordance with one embodiment;

FIGS. 2A to 2C schematically illustrate normal vision, blurred vision, and corrected vision in accordance with one embodiment, respectively;

FIGS. 3A and 3B are schematic diagrams of a light field display in which respective pixel subsets are aligned to emit light through a corresponding microlens or lenslet, in accordance with one embodiment;

FIGS. 4A to 4C are schematic diagrams of exemplary light field vision testing or previewing systems (e.g. refractors/phoropters), in accordance with different embodiments;

FIG. 5 is a plot of the angular resolution of an exemplary light field display as a function of the dioptric power generated, in accordance with one embodiment;

FIGS. 6A to 6D are schematic plots of the image quality generated by a light field refractor/phoropter as a function of the dioptric power generated by using in combination with the light field display (A) no refractive component, (B) one refractive component, (C) and (D) a multiplicity of refractive components;

FIGS. 7A, 7B and 7C are perspective views of exemplary light field refractors/phoropters, showing a casing thereof in cross-section (A and B) and a unit combining side-by-side two of the units (C) shown in 7A and 7B, in accordance with one embodiment;

FIG. 8 is a process flow diagram of an exemplary dynamic subjective vision testing method, in accordance with one embodiment;

FIG. 9 is a schematic diagram of an exemplary light field image showing two columns of optotypes at different dioptric power for the method of FIG. 8, in accordance with one embodiment;

FIG. 10 is a schematic diagram of an exemplary vision testing system configuration employing a microlens array, in accordance with at least one embodiment;

FIG. 11 is a schematic diagram of an exemplary pixel and lenslet system, in accordance with various embodiments;

FIG. 12 is a schematic diagram illustrating a focusing of a rectangular beam, in accordance with some embodiments;

FIG. 13 is an exemplary plot illustrating a spacing of retinal beam spots as a function of various parameters, in accordance with at least one embodiment;

FIG. 14 is an exemplary plot illustrating an interplay of various parameters in an exemplary light field system, in accordance with some embodiments;

FIGS. 15A and 15B are exemplary plots of a minimum beam size in an exemplary light field display system as a function of light field shaping layer focal length, with the condition that interference from different lenslets is avoided, and FIGS. 15C and 15D are exemplary plots of a minimum beam size in the exemplary light field display system of FIGS. 15A and 15B, without the condition that interference from different lenslets is avoided, in accordance with various embodiments;

FIGS. 16A and 16B are exemplary plots illustrating the effect of lenslet size on retinal spot size, in accordance with various embodiments;

FIGS. 17A and 17C, and FIGS. 17B and 17D, are exemplary plots illustrating the field of view and retina spread, respectively, for an exemplary light field display system, in accordance with some embodiments;

FIGS. 18A and 18B are schematic diagrams illustrating an exemplary light field display system comprising a dynamic light field shaping layer, in accordance with various embodiments;

FIG. 19 is a schematic diagram illustrating an exemplary process for displaying adjusted content using a dynamic light field shaping layer, in accordance with some embodiments;

FIGS. 20A to 201 are exemplary plots illustrating various parameters for an exemplary light field system, in accordance with one embodiment;

FIGS. 21A to 21J are exemplary plots illustrating various parameters for another exemplary light field system, in accordance with one embodiment;

FIGS. 22A to 22J are exemplary plots illustrating various parameters for another exemplary light field system, in accordance with one embodiment;

FIGS. 23A to 23J are exemplary plots illustrating various parameters for another exemplary light field system, in accordance with one embodiment;

FIGS. 24A and 24B are perspective views of an exemplary portable cognitive impairment assessment system, in accordance with one embodiment,

FIGS. 25A and 25B are schematic diagrams illustrating an exemplary cognitive impairment assessment device comprising a user-interfacing portion and a load-bearing portion and comprising a displaceable light field shaping layer, in accordance with one embodiment;

FIGS. 26A to 26J are exemplary plots illustrating various parameters for another exemplary light field system, in accordance with one embodiment; and

FIGS. 27A to 27K are exemplary plots illustrating various parameters for another exemplary light field system, in accordance with one embodiment.

Elements in the several figures are illustrated for simplicity and clarity and have not necessarily been drawn to scale. For example, the dimensions of some of the elements in the figures may be emphasised relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

Unless defined otherwise, all technical and scientific terms used herein have the same meaning as commonly understood by one of ordinary skill in the art to which this invention belongs.

Throughout the specification and claims, the following terms take the meanings explicitly associated herein, unless the context clearly dictates otherwise. The phrase “in one of the embodiments” or “in at least one of the various embodiments” as used herein does not necessarily refer to the same embodiment, though it may. Furthermore, the phrase “in another embodiment” or “in some embodiments” as used herein does not necessarily refer to a different embodiment, although it may. Thus, as described below, various embodiments may be readily combined, without departing from the scope or spirit of the innovations disclosed herein.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The term “comprising” as used herein will be understood to mean that the list following is non-exhaustive and may or may not include any other additional suitable items, for example one or more further feature(s), component(s) and/or element(s) as appropriate.

The systems and methods described herein provide, in accordance with different embodiments, different examples of a light field display, adjusted pixel rendering method therefor, and adjusted vision perception system and method using same. For example, some of the herein-described embodiments provide improvements or alternatives to current light field display technologies, for instance, in providing a range of dioptric corrections that may be displayed or a given light field display system having a particular light field shaping layer geometry and display resolution. Various embodiments relate to the provision of an increased range of perception adjustments accessible to, for instance, a vision testing system (e.g. a refractor or phoropter), or other display system operable to provide a perception adjustment through the provision of a light field, such as a smart phone, television, pixelated display, car dashboard interface, or the like.

These and other such applications will be described in further detail below. For example, devices, displays and methods described herein may allow a user's perception of one or more input images (or input image portions), where each image or image portion is virtually located or perceived to be at a distinct image plane/depth location, to be adjusted or altered using the described light field display technology, again allowing for, for instance, corrective assessment, or the display of media content (e.g. images or videos) in accordance with a dioptric shift or perception adjustment that may not be enabled by a light field display technology having a fixed geometry or configuration. According, some of the herein-described embodiments provide a light field display for rendering perception-adjusted content in which a geometry, disposition and/or relative positioning of an integrated or cooperative light field shaping element array (e.g. light field shaping layer (LFSL)) can be dynamically adjusted to improve or optimize image rendering and perception adjustment capabilities (e.g. adjustment range, resolution, brightness, and/or overall quality).

Some of the herein described embodiments provide for digital display devices, or devices encompassing such displays, for use by users having reduced visual acuity, whereby images ultimately rendered by such devices can be dynamically processed to accommodate the user's reduced visual acuity so that they may comfortably consume rendered images without the use of corrective eyewear, contact lenses, or surgical intervention, as would otherwise be required. As noted above, embodiments are not to be limited as such as the notions and solutions described herein may also be applied to other technologies in which a user's perception of an input image to be displayed can be altered or adjusted via the light field display. Again, similar implementation of the herein described embodiments can allow for implementation of digitally adaptive vision tests or corrective or adaptive vision previews or simulations such that individuals with such reduced visual acuity can be exposed to distinct perceptively adjusted versions of an input image(s) to subjectively ascertain a potentially required or preferred vision correction.

Generally, digital displays as considered herein will comprise a set of image rendering pixels and a corresponding set of light field shaping elements that at least partially govern a light field emanated thereby to produce a perceptively adjusted version of the input image, notably distinct perceptively adjusted portions of an input image or input scene, which may include distinct portions of a same image, a same 2.5D/3D scene, or distinct images (portions) associated with different image depths, effects and/or locations and assembled into a combined visual input. For simplicity, the following will generally consider distinctly addressed portions or segments as distinct portions of an input image, whether that input image comprises a singular image having distinctly characterised portions, a digital assembly of distinctly characterised images, overlays, backgrounds, foregrounds or the like, or any other such digital image combinations.

In some examples, light field shaping elements may take the form of a light field shaping layer or like array of optical elements to be disposed relative to the display pixels in at least partially governing the emanated light field. As described in further detail below, such light field shaping layer elements may take the form of a microlens and/or pinhole array, or other like arrays of optical elements, or again take the form of an underlying light field shaping layer, such as an underlying array of optical gratings or like optical elements operable to produce a directional pixelated output.

Within the context of a light field shaping layer, as described in further detail below in accordance with some embodiments, the light field shaping layer may be disposed at a preset or adjustable distance from the pixelated display so to controllably shape or influence a light field emanating therefrom. For instance, each light field shaping layer can be defined by an array of optical elements centered over a corresponding subset of the display's pixel array to optically influence a light field emanating therefrom and thereby govern a projection thereof from the display medium toward the user, for instance, providing some control over how each pixel or pixel group will be viewed by the viewer's eye(s). As will be further detailed below, arrayed optical elements may include, but are not limited to, lenslets, microlenses or other such diffractive optical elements that together form, for example, a lenslet array; pinholes or like apertures or windows that together form, for example, a parallax or like barrier; concentrically patterned barriers, e.g. cut outs and/or windows, such as a to define a Fresnel zone plate or optical sieve, for example, and that together form a diffractive optical barrier; and/or a combination thereof, such as for example, a lenslet array whose respective lenses or lenslets are partially shadowed or barriered around a periphery thereof so to combine the refractive properties of the lenslet with some of the advantages provided by a pinhole barrier.

In operation, the display device may also generally invoke a hardware processor operable on image pixel (or subpixel) data for an image to be displayed to output corrected or adjusted image pixel data to be rendered as a function of a stored characteristic of the light field shaping elements and/or layer (e.g. layer distance from display screen, distance between optical elements (pitch), absolute relative location of each pixel or subpixel to a corresponding optical element, properties of the optical elements (size, diffractive and/or refractive properties, etc.), or other such properties, and a selected vision correction or adjustment parameter related to the user's reduced visual acuity or intended viewing experience. Image processing may, in some embodiments, be dynamically adjusted as a function of the user's visual acuity or intended application so to actively adjust a distance of a virtual image plane, or perceived image on the user's retinal plane given a quantified user eye focus or like optical aberration(s), induced upon rendering the corrected/adjusted image pixel data via the optical layer and/or elements, for example, or otherwise actively adjust image processing parameters as may be considered, for example, when implementing a viewer-adaptive pre-filtering algorithm or like approach (e.g. compressive light field optimization), so to at least in part govern an image perceived by the user's eye(s) given pixel or subpixel-specific light visible thereby through the layer.

The skilled artisan will appreciate that various ray tracing processes may be employed, in accordance with various embodiments, for rendering, for instance, adjusted content to accommodate a user's reduced visual acuity. For example, various embodiments relate to computationally implementing one or more of the ray tracing processes described in Applicant's U.S. Pat. No. 10,394,322 issued Aug. 27, 2019, U.S. Pat. No. 10,636,116 issued Apr. 28, 2020, and/or U.S. Pat. No. 10,761,604 issued Sep. 1, 2020, the entire contents of which are hereby incorporated herein by reference. Similarly, while various embodiments herein described employ a LFSL layer disposed parallel to a display screen, the skilled artisan will appreciate that ray tracing with non-parallel planes, as described in, for instance, some of the above noted patents, the entire contents of which are hereby incorporated herein by reference, is also herein considered, in accordance with various embodiments. Various embodiments may, additionally or alternatively, relate to self-identifying light field display systems and processes, whereby a user may self-identify themselves for display of preferred content (e.g. content displayed with a perception adjustment corresponding to a particular dioptric adjustment) while, for instance, maintaining user privacy. Such embodiments are further described in co-pending U.S. Patent Application No. 63/056,188, the entire contents of which are also hereby incorporated herein by reference.

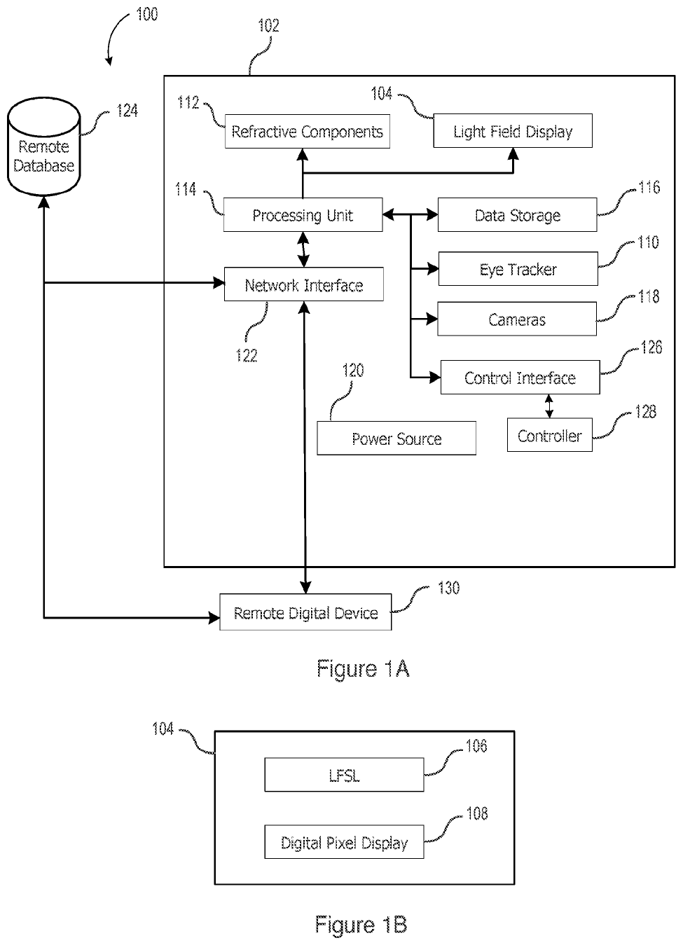

While various embodiments may apply to various configurations of light field display systems known in the art, exemplary light field display systems in which a dynamic light field shaping layer as described herein may apply will be described with reference to exemplary vision testing systems (FIGS. 1A, 1B, 4A to 4C, and 7 to 10). Such examples are not intended to limit the scope of the systems and methods herein described, and are included to provide context, only, for non-limiting exemplary light field display systems and methods that may employ a dynamic light field shaping layer.

With reference to FIGS. 1A and 1B, and in accordance with different embodiments, an exemplary subjective vision testing device/system (interchangeably referred to as a corrective vision previewing device/system), generally referred to using the numeral 100, will now be described. At the heart of this exemplary system is a light field vision testing device such as a light field refractor or phoropter device 102. Generally, light field refractor 102 is a device comprising, as mentioned above, a light field display 104 and which is operable to display or generate one or more images, including optotypes, to a user or patient having his/her vision acuity (e.g. refractive error) tested.

In some embodiments, as illustrated in FIG. 1B, light field display 104 comprises a light field shaping layer (LFSL) 108 overlaid or placed in front of a digital pixel display 110 (i.e. LCD, LED, OLED, etc.). For the sake of illustration, the following embodiments will be described within the context of a LFSL 108 defined, at least in part, by a lenslet array comprising an array of microlenses (also interchangeably referred to herein as lenslets) that are each disposed at a distance from a corresponding subset of image rendering pixels in an underlying digital display. It will be appreciated that while a light field shaping layer may be manufactured and disposed as a digital screen overlay, other integrated concepts may also be considered, for example, where light field shaping elements such as a textured or masked glass plate, beam-shaping light sources (e.g. directional light sources and/or backlit integrated optical grating array), or like component, may be coupled with integrally formed or manufactured integral components of a digital display system.

Accordingly, each lenslet will predictively shape light emanating from these pixel subsets to at least partially govern light rays being projected toward the user by the display device. As noted above, other light field shaping layers may also be considered herein without departing from the general scope and nature of the present disclosure, whereby light field shaping will be understood by the person of ordinary skill in the art to reference measures by which light, that would otherwise emanate indiscriminately (i.e. isotropically) from each pixel group, is deliberately controlled to define predictable light rays that can be traced between the user and the device's pixels through the shaping layer.

For greater clarity, a light field is generally defined as a vector function that describes the amount of light flowing in every direction through every point in space. In other words, anything that produces or reflects light has an associated light field. The embodiments described herein produce light fields from an object that are not “natural” vector functions one would expect to observe from that object. This gives it the ability to emulate the “natural” light fields of objects that do not physically exist, such as a virtual display located far behind the light field display.

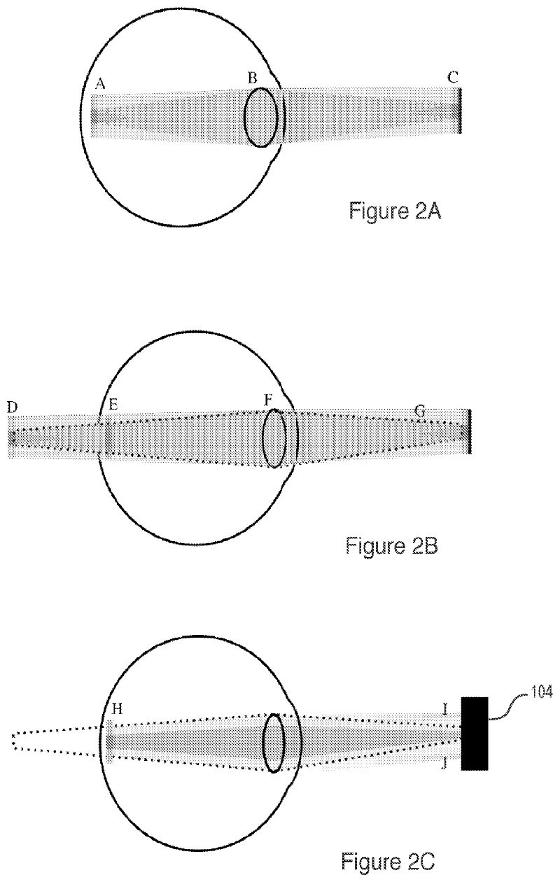

In one example, to apply this technology to vision correction, consider first the normal ability of the lens in an eye, as schematically illustrated in FIG. 2A, where, for normal vision, the image is to the right of the eye (C) and is projected through the lens (B) to the retina at the back of the eye (A). As comparatively shown in FIG. 2B, the poor lens shape and inability to accommodate (F) in presbyopia causes the image to be focused past the retina (D) forming a blurry image on the retina (E). The dotted lines outline the path of a beam of light (G). Naturally, other optical aberrations present in the eye will have different impacts on image formation on the retina. To address these aberrations, a light field display 104, in accordance with some embodiments, projects the correct sharp image (H) on the retina for an eye with a crystalline lens which otherwise could not accommodate sufficiently to produce a sharp image. The other two light field pixels (I) and (J) are drawn lightly, but would otherwise fill out the rest of the image.

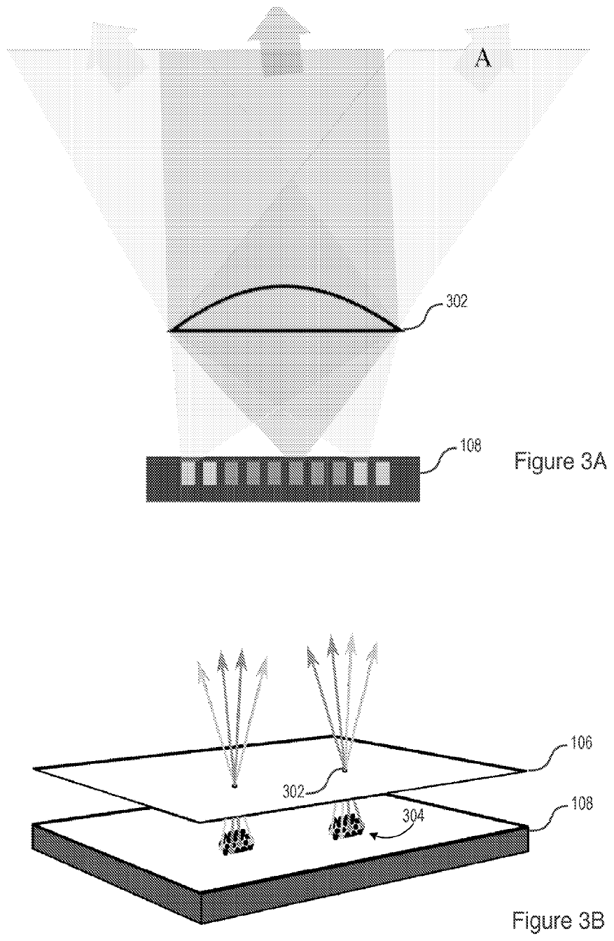

As will be appreciated by the skilled artisan, a light field as seen in FIG. 2C cannot be produced with a ‘normal’ two-dimensional display because the pixels' light field emits light isotopically. Instead it is necessary to exercise tight control on the angle and origin of the light emitted, for example, using a microlens array or other light field shaping layer such as a parallax barrier, or combination thereof. Following with the example of a microlens array for LFSL 106, FIG. 3A schematically illustrates a single light field pixel defined by a convex microlens 302 disposed at its focus from a corresponding subset of pixels in a digital pixel display 108 to produce a substantially collimated beam of light emitted by these pixels, whereby the direction of the beam is controlled by the location of the pixel(s) relative to the microlens. The single light field pixel produces a beam similar to that shown in FIG. 2C where the outside rays are lighter and the majority inside rays are darker. The digital pixel display 108 emits light which hits the microlens 302 and it results in a beam of substantially collimated light (A).

Accordingly, upon predictably aligning a particular microlens array with a pixel array, a designated “circle” of pixels will correspond with each microlens and be responsible for delivering light to the pupil through that lens. FIG. 3B schematically illustrates an example of a light field display assembly in which a LFSL 106 sits above a pixel display 108 to have pixels 304 emit light through the microlens array. A ray-tracing algorithm can thus be used to produce a pattern to be displayed on the pixel array below the microlens in order to create the desired virtual image that will effectively correct for the viewer's reduced visual acuity.

As will be detailed further below, the separation between the LFSL 106 and the pixel array 108, as well as the pitch of the lenses, can be selected as a function of various operating characteristics, such as the normal or average operating distance of the display, and/or normal or average operating ambient light levels.

In some embodiments, LFSL 106 may be a microlens array (MLA) defined by a hexagonal array of microlenses or lenslet disposed so to overlay a corresponding square pixel array of digital pixel display 108. In doing so, while each microlens can be aligned with a designated subset of pixels to produce light field pixels as described above, the hexagonal-to-square array mismatch can alleviate certain periodic optical artifacts that may otherwise be manifested given the periodic nature of the optical elements and principles being relied upon to produce the desired optical image corrections. Conversely, other geometries, such as a square microlens array, or an array comprising elongated hexagonal lenslets, may be favoured when operating a digital display comprising a hexagonal pixel array.

In some embodiments, the MLA may further or alternatively be overlaid or disposed at an angle (rotation) relative to the underlying pixel array, which can further or alternatively alleviate period optical artifacts.

In yet some further or alternative embodiments, a pitch ratio between the microlens array and pixel array may be deliberately selected to further or alternatively alleviate periodic optical artifacts. For example, a perfectly matched pitch ratio (i.e. an exact integer number of display pixels per microlens) is most likely to induce periodic optical artifacts, whereas a pitch ratio mismatch can help reduce such occurrences.

Accordingly, in some embodiments, the pitch ratio will be selected to define an irrational number, or at least, an irregular ratio, so to minimise periodic optical artifacts. For instance, a structural periodicity can be defined so to reduce the number of periodic occurrences within the dimensions of the display screen at hand, e.g. ideally selected so to define a structural period that is greater than the size of the display screen being used.

While this example is provided within the context of a microlens array, similar structural design considerations may be applied within the context of a parallax barrier, diffractive barrier or combination thereof. In some embodiments, light field display 104 can render dynamic images at over 30 frames per second on the hardware in a smartphone.

Accordingly, a display device as described above and further exemplified below, can be configured to render a corrected or adjusted image via the light field shaping layer that accommodates, tests or simulates for the user's visual acuity. By adjusting the image correction in accordance with the user's actual predefined, set or selected visual acuity level, different users and visual acuity may be accommodated using a same device configuration, whereas adjusting such parameters for a given user may allow for testing for or simulation of different corrective or visual adjustment solutions. For example, by adjusting corrective image pixel data to dynamically adjust a virtual image distance below/above the display as rendered via the light field shaping layer, different visual acuity levels may be accommodated, and that, for an image input as a whole, for distinctly various portions thereof, or again progressively across a particular input.

As noted in the examples below, in some embodiments, light field rendering may be adjusted to effectively generate a virtual image on a virtual image plane that is set at a designated distance from an input user pupil location, for example, so to effectively push back, or move forward, a perceived image, or portion thereof, relative to the light field refractor device 102. In yet other embodiments, light field rendering may rather or alternatively seek to map the input image on a retinal plane of the user, taking into account visual aberrations, so to adaptively adjust rendering of the input image on the display device to produce the mapped effect. Namely, where the unadjusted input image would otherwise typically come into focus in front of or behind the retinal plane (and/or be subject to other optical aberrations), this approach allows to map the intended image on the retinal plane and work therefrom to address designated optical aberrations accordingly. Using this approach, the device may further computationally interpret and compute virtual image distances tending toward infinity, for example, for extreme cases of presbyopia. This approach may also more readily allow, as will be appreciated by the below description, for adaptability to other visual aberrations that may not be as readily modeled using a virtual image and image plane implementation. In both of these examples, and like embodiments, the input image is digitally mapped to an adjusted image plane (e.g. virtual image plane or retinal plane) designated to provide the user with a designated image perception adjustment that at least partially addresses designated visual aberrations. Naturally, while visual aberrations may be addressed using these approaches, other visual effects may also be implemented using similar techniques.

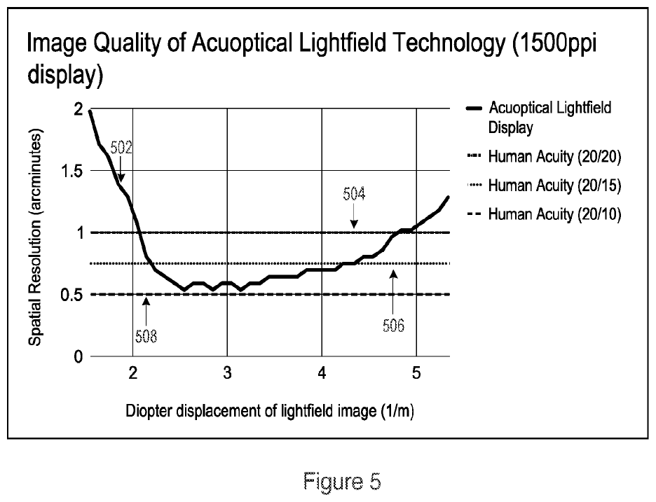

As an example of the effectiveness of the light field display in generating a diopter displacement (e.g. simulate the effect of looking through an optical component (i.e. a lens) of a given diopter strength or power) is shown in FIG. 5, where a plot is shown of the angular resolution (in arcminutes) of an exemplary light field display comprising a 1500 ppi digital pixel display, as a function of the dioptric power of the light field image (in diopters). From this plot, it is clear that, in this particular example employing a static LFSL, the light field display is able to generate displacements (line 502) in diopters that have higher resolution corresponding to 20/20 vision (line 504) or better (e.g. 20/15—line 506) and close to (20/10—line 508)), here within a dioptric power range of 2 to 2.5 diopters.

Thus, in the context of a refractor 102, light field display 104 (in conjunction with light field rendering or ray-tracing methods referenced above) may, according to different embodiments, be used to replace, at least in part, traditional optical components.

In some embodiments, the light field display can display a virtual image at optical infinity, meaning that any level of accommodation-based presbyopia (e.g. first order) can be corrected for. In some further embodiments, the light field display can both push the image back or forward, thus allowing for selective image corrections for both hyperopia (far-sightedness) and myopia (nearsightedness). In yet further embodiments, variable displacements and/or accommodations may be applied as a function of non-uniform visual aberrations, or again to provide perceptive previewing or simulation of non-uniform or otherwise variable corrective powers/measures across a particular input or field of view.

However, the light field rendering system introduced above, in conjunction with various ray-tracing methods as referenced above, may also be used with other devices which may similarly comprise a light field display. For example, this may include a smartphone, tablets, e-readers, watches, televisions, GPS devices, laptops, desktop computer monitors, televisions, smart televisions, handheld video game consoles and controllers, vehicular dashboard and/or entertainment displays, and the like, without limitation.

Accordingly, any light field processing or ray-tracing methods as referenced herein, and related light field display solutions, can be equally applied to image perception adjustment solutions for visual media consumption, as they can for subjective vision testing solutions, or other technologically related fields of endeavour. As alluded to above, the light field display and rendering/ray-tracing methods discussed above may all be used to implement, according to various embodiments, a subjective vision testing device or system such as a phoropter or refractor. Indeed, a light field display may replace, at least in part, the various refractive optical components usually present in such a device. Thus, vision correction light field ray tracing methods may equally be applied to render optotypes at different dioptric power or refractive correction by generating vision correction for hyperopia (far-sightedness) and myopia (nearsightedness), as was described above in the general case of a vision correction display. Light field systems and methods described herein, according to some embodiments, may be applied to create the same capabilities as a traditional instrument and to open a spectrum of new features, all while improving upon many other operating aspects of the device. For example, the digital nature of the light field display enables continuous changes in dioptric power compared to the discrete change caused by switching or changing a lens or similar; displaying two or more different dioptric corrections seamlessly at the same time; and, in some embodiments, the possibility of measuring higher-order aberrations and/or to simulate them for different purposes such as, deciding for free-form lenses, cataract surgery operation protocols, IOL choice, etc.

Going back to FIG. 1A, as producing a light field with angular resolution sufficient for accommodation correction over the full viewing ‘zone’ of a display would generally require an astronomically high pixel density, instead, a correct light field can be produced, in some embodiments, only at or around the location of the user's pupil(s). To do so, where a location or position of the user's eye is not otherwise rigidly constrained (e.g. within the context of a subject eye testing device or the like) the light field display can be paired with pupil tracking technology, as will be discussed below, to track a location of the user's eyes/pupils relative to the display. The display can then compensate for the user's eye location and produce the correct virtual image, for example, in real-time. Thus, in some embodiments, light field refractor 102 may include, integrated therein or interfacing therewith, a pupil/eye tracking system 110 to improve or enhance corrective image rendering by tracking a location of the user's eye(s)/pupil(s) (e.g. both or one, e.g. dominant, eye(s)) and adjusting light field corrections accordingly one or more eye/pupil tracking light sources, such as one or more infrared (IR) or near-IR (NIR) light source(s) to accommodate operation in limited ambient light conditions, leverage retinal retro-reflections, invoke corneal reflection, and/or other such considerations. For instance, different IR/NIR pupil tracking techniques may employ one or more (e.g. arrayed) directed or broad illumination light sources to stimulate retinal retro-reflection and/or corneal reflection in identifying a tracking a pupil location. Other techniques may employ ambient or IR/NIR light-based machine vision and facial recognition techniques to otherwise locate and track the user's eye(s)/pupil(s). To do so, one or more corresponding (e.g. visible, IR/NIR) cameras may be deployed to capture eye/pupil tracking signals that can be processed, using various image/sensor data processing techniques, to map a 3D location of the user's eye(s)/pupil(s). As mentioned above, in some embodiments, such eye/pupil tracking hardware/software may be integral to device 102, for instance, operating in concert with integrated components such as one or more front facing camera(s), onboard IR/NIR light source(s) (not shown) and the like. In other user environments, such as in a vehicular environment, eye/pupil tracking hardware may be further distributed within the environment, such as dash, console, ceiling, windshield, mirror or similarly-mounted camera(s), light sources, etc.

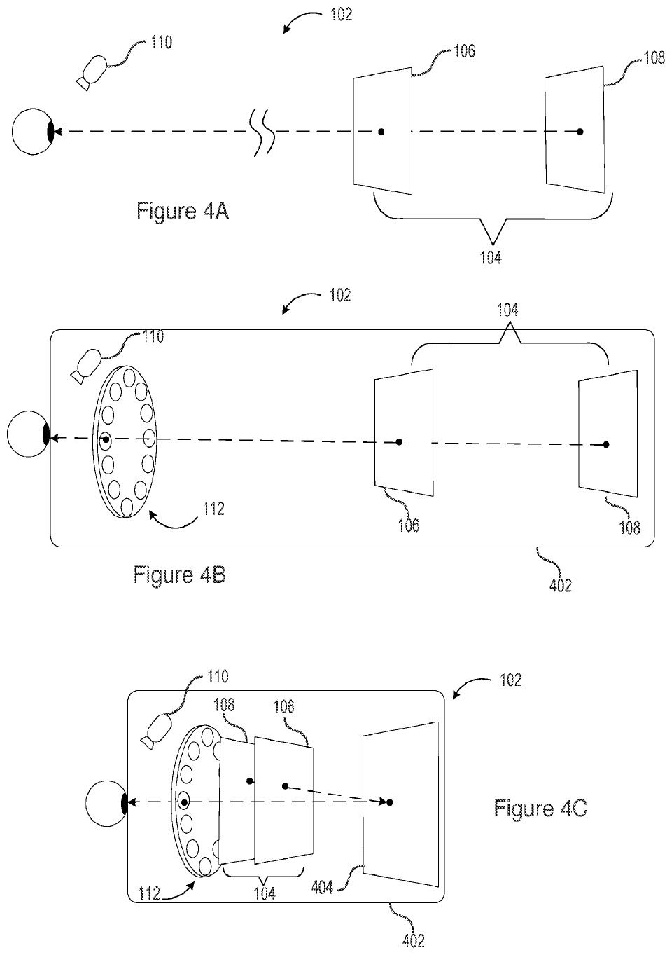

In one embodiment and as illustrated in FIG. 4A, light field refractor 102 may be configured with light field display 104 located relatively far away (e.g. one or more meters) from the user's eye currently being diagnosed. Note that the pointed line in FIGS. 4A to 4C is used to schematically illustrate the direction of the light rays emitted by light field display 104. Also illustrated is eye-tracker 110, which may be provided as a physically separate element, for example, installed at a given location in a room or similar. In some embodiments, the noted eye/pupil tracker 110 may include the projection of IR markers/patterns to help align the patient's eye with the light field display. In some embodiments, a tolerance window (e.g. “eye box”) may be considered to limit the need to refresh the ray-tracing iteration. An exemplary value of the size of the eye box, in some embodiments, is around 6 mm, though smaller (e.g. 4 mm) or larger eye boxes may alternatively be set to impact image quality, stability or like operational parameters.

Going back to FIG. 1A, light field refractor 102 may also comprise, according to different embodiments and as will be further discussed below, one or more refractive optical components 112, a processing unit 114, a data storage unit or internal memory 116, one or more cameras 118, a power source 120, a network interface 122 for communicating via network to a remote database or server 124.

In some embodiments, power source 120 may comprise, for example, a rechargeable Li-ion battery or similar. In some embodiments, it may comprise an additional external power source, such as, for example, a USB-C external power supply. It may also comprise a visual indicator (screen or display) for communicating the device's power status, for example whether the device is on/off or recharging.

In some embodiments, internal memory 116 may be any form of electronic storage, including a disk drive, optical drive, read-only memory, random-access memory, or flash memory, to name a few examples. In some embodiments, a library of chart patterns (Snellen charts, prescribed optotypes, forms, patterns, or other) may be located in internal memory 116 and/or retrievable from remote server 124 via network interface 122.

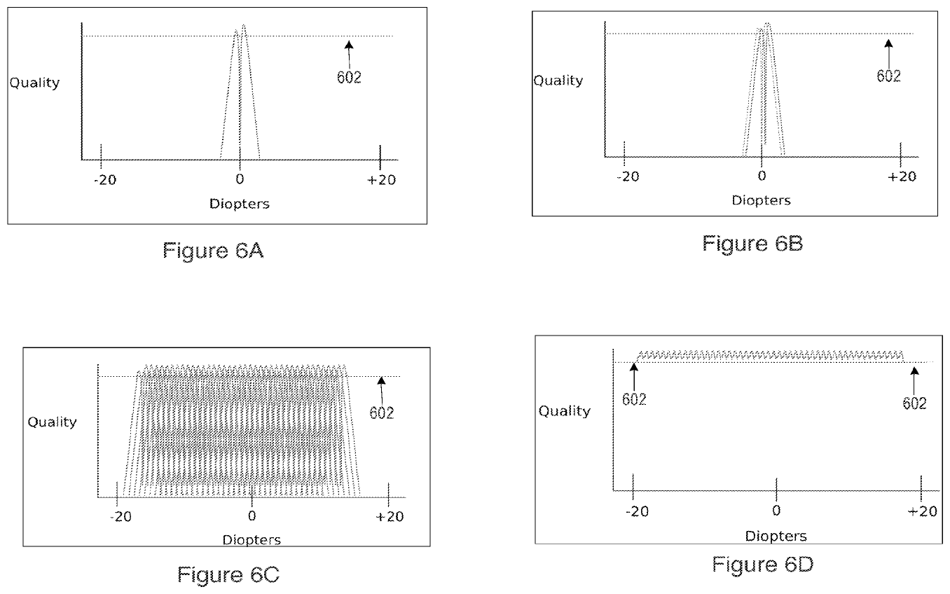

In some embodiments, one or more optical components 112 may be used in combination with the light field display 104, for example to shorten the size of refractor 102 and still offer an acceptable range in dioptric power. The general principle is schematically illustrated in the plots of FIGS. 6A to 6D. In these schematic plots, the image quality (e.g. inverse of the angular resolution, higher is better) at which optotypes are small enough to be useful for vision testing in this plot is above horizontal line 602 which represents typical 20/20 vision. FIG. 6A shows the plot for the light field display having a static LFSL only, where we see the characteristic two peaks corresponding to the smallest resolvable point, one of which was plotted in FIG. 5 (here inverted and shown as a peak instead of a basin), and where each region above the line may cover a few diopters of dioptric power, according to some embodiments. While the dioptric range may, in some embodiments, be more limited than needed when relying only on the light field display with a static LFSL, it is possible to shift this interval by adding one or more refractive optical components. This is shown in FIG. 6B where the regions above the line 602 is shifted to the left (negative diopters) by adding a single lens in the optical path.

Thus, by using a multiplicity of refractive optical components 112 or by alternating sequentially between different refractive components 112 of increasing or decreasing dioptric power, it is possible to shift the center of the light field diopter range to any required value, as shown in FIG. 6C, and thus the image quality may be kept above line 602 for any required dioptric power as shown in FIG. 6D. In some embodiments, a range of 30 diopters from +10 to-20 may be covered for example. In the case of one or more reels of lenses, the lens may be switched for a given larger dioptric power increment, and the light field display would be used to provide a finer continuous change to accurately pin-point the required total dioptric power required to compensate for the patient's reduced visual acuity. This would still result in light field refractor 102 having a reduced number of refractive optical components compared to the number of components needed in a traditional refractor, while drastically enhancing the overall fine-tuning ability of the device.

One example, according to one embodiment, of such a light field refractor 102 is schematically illustrated in FIG. 4B, wherein the light field display 104 (herein shown comprising LFSL 106 and digital pixel display 108) is combined with a multiplicity of refractive components 112 (herein illustrated as a reel of lenses as an example only). By changing the refractive component used in combination with light field display 104 with a static LFSL, a larger dioptric range may be covered. This may also provide means to reduce the dimension of device 102 as mentioned above, making it more portable, so that all its internal components may be encompassed into a shell, housing or casing 402. In some embodiments, light field refractor 102 may thus comprise a durable ABS housing that may be shock and harsh-environment resistant. In some embodiments, light field refractor 102 may also comprise a telescopic feel for fixed or portable usage; optional mounting brackets, and/or a carrying case (not shown). In some embodiments, all components may be internally protected and sealed from the elements.

In some embodiments, casing 402 may further comprise a head-rest or similar (not shown) to keep the user's head still and substantially in the same location, thus, in such examples, foregoing the general utility of a pupil tracker or similar techniques by substantially fixing a pupil location relative to this headrest.

In some embodiments, it may also be possible to further reduce the size of device 102 by adding, for example, a mirror or any device which may increase the optical path. This is illustrated in FIG. 4C where the length of the device was reduced by adding a mirror 404. This is shown schematically by the pointed arrow which illustrates the light being emitted from pixel display 108 travelling through LFSL 106 before being reflected back by mirror through refractive components 112 and ultimately hitting the eye.

The skilled technician will understand that different examples of refractive components 112 may be include, without limitation, one or more lenses, sometimes arranged in order of increasing dioptric power in one or more reels of lenses similar to what is typically found in traditional refractors/phoropters; an electrically controlled fluid lens; active Fresnel lens; and/or Spatial Light Modulators (SLM). In some embodiments, additional motors and/or actuators (not shown) may be used to operate refractive components 112. The motors/actuators may be communicatively linked to processing unit 114 and power source 120, and operate seamlessly with light field display 102 to provide the required dioptric power.

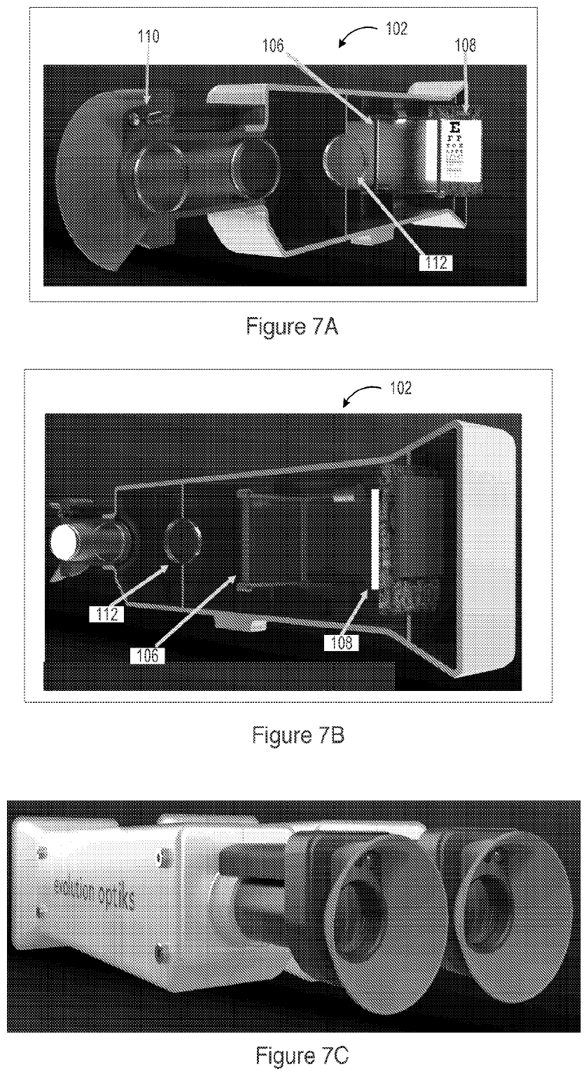

For example, FIGS. 7A and 7B show a perspective view of an exemplary light field phoropter 102 similar to the one schematically shown in FIG. 3B, but wherein the refractive component 112 is an electrically tunable liquid lens. In some embodiments, the electrically tunable lens may have a range of ±13 diopters. In accordance with other embodiments, one or more refractive components 112 may be removed from the system 102, as the dioptric range enabled by the refractive components 112 may be provided through mechanical displacement of a LFSL, as discussed below. That is, as further detailed below, the dynamic range of the light field display solution can be significantly increased by dynamically adjusted a relative distance of the LFSL, such as a microlens array, relative to the pixelated display such that, in combination with a correspondingly adaptive ray tracing process, a greater perceptive adjustment range can be achieved (e.g. greater perceived dioptric correction range can be implemented).

In one illustrative embodiment, a 1000 dpi display is used with a MLA having a 65 mm focal distance and 1000 μm pitch with the user's eye located at a distance of about 26 cm. A similar embodiment uses the same MLA and user distance with a 3000 dpi display.

Other displays having resolutions including 750 dpi, 1000 dpi, 1500 dpi and 3000 dpi may also be used, as may be MLAs with a focal distance and pitch of 65 mm and 1000 μm, 43 mm and 525 μm, 65 mm and 590 μm, 60 mm and 425 μm, 30 mm and 220 μm, and 60 mm and 425 μm, respectively, and user distances of 26 cm, 45 cm or 65 cm.

Going back to FIG. 1A, in some embodiments, eye-tracker 110 may further comprise a digital camera, in which case it may be used to further acquire images of the user's eye to provide further diagnostics, such as pupillary reflexes and responses during testing for example. In other embodiments, one or more additional cameras 118 may be used to acquire these images instead. In some embodiments, light field refractor 102 may comprise built-in stereoscopic tracking cameras.

In some embodiments, feedback and/or control of the vision test being administered by system 100 may be given via a control interface 126. In some embodiments, the control interface 126 may comprise a dedicated handheld controller-like device 128. This controller 128 may be connected via a cable or wirelessly, and may be used by the patient directly and/or by an operator like an eye professional. In some embodiments, both the patient and operator may have their own dedicated controller 128. In some embodiments, the controller may comprise digital buttons, analog thumbstick, dials, touch screens, and/or triggers.

In some embodiments, control interface 126 may comprise a digital screen or touch screen, either on refractor 102 itself or part of an external module (not shown). In other embodiments, control interface 126 may let on or more external remote devices (i.e. computer, laptop, tablet, smartphone, remote, etc.) control light field refractor 102 via network interface 122. For example, remote digital device 130 may be connected to light field refractor 102 via a cable (e.g. USB cable, etc.) or wirelessly (e.g. via Wi-Fi, Bluetooth or similar) and interface with light field refractor 102 via a dedicated application, software or website (not shown). Such a dedicated application may comprise a graphical user interface (GUI), and may also be communicatively linked to remote database 124.

In some embodiments, the user or patient may give feedback verbally and the operator may control the vision test as a function of that verbal feedback. In some embodiments, refractor 102 may comprise a microphone (not shown) to record the patient's verbal communications, either to communicate them to a remote operator via network interface 122 or to directly interact with the device (e.g. via speech recognition or similar).

Going back to FIG. 1A, processing unit 114 may be communicatively connected to data storage 116, eye tracker 110, light field display 104 and refractive components 112. Processing unit 114 may be responsible for rendering one or more images or optotypes via light field display 104 and, in some embodiments, jointly control refractive components 112 or a LFSL 106 position to achieve a required total change in dioptric power. It may also be operable to send and receive data to internal memory 116 or to/from remote database 124 via network interface 122.

In some embodiments, diagnostic data may be automatically transmitted/communicated to remote database 124 or remote digital device 130 via network interface 122 through the use of a wired or wireless network connection. The skilled artisan will understand that different means of connecting electronic devices may be considered herein, such as, but not limited to, Wi-Fi, Bluetooth, NFC, Cellular, 2G, 3G, 4G, 5G or similar. In some embodiments, the connection may be made via a connector cable (e.g. USB including microUSB, USB-C. Lightning connector, etc.). In some embodiments, remote digital device 130 may be located in a different room, building or city.

In some embodiments, two light field refractors 102 may be combined side-by-side to independently measure the visual acuity of both left and right eye at the same time. An example is shown in FIG. 7C, where two units corresponding to the embodiment of FIG. 7A or 7B (used as an example only) are placed side-by-side or fused into a single device.

In some embodiments, a dedicated application, software or website may provide integration with third party patient data software. In some embodiments, software required to operate and installed on refractor 102 may be updated on-the-fly via a network connection and/or be integrated with the patient's smartphone app for updates and reminders.

In some embodiments, the dedicated application, software or website may further provide a remote, real-time collaboration platform between an eye professional and user/patient, and/or between different eye professionals. This may include interaction between different participants via video chat, audio chat, text messages, etc.

In some embodiments, light field refractor 102 may be self-operated or operated by an optometrist, ophthalmologist or other certified eye-care professional. For example, in some embodiments, a user/patient may use refractor 102 in the comfort of his/her own home, in a store or a remote location.

In accordance with various embodiments, the light field display 102 may comprise various forms of systems for performing vision-based tests. For instance, while some embodiments described above relate to the light field display 102 comprising a refractor or phoropter for assessing a user's visual acuity, various other embodiments relate to a light field system 102 operable to perform, for instance, a cognitive impairment assessment. In some embodiments, the light field system 102 may display, for instance, moving content to assess a user's ability to track motion, or perform any number of other cognitive impairment evaluations known in the art, such as those related to saccadic movement and/or fixation. Naturally, such embodiments may further relate to light field display systems that are further operable to track a user's gaze and/or eye movement and store data related thereto for further processing and assessment.

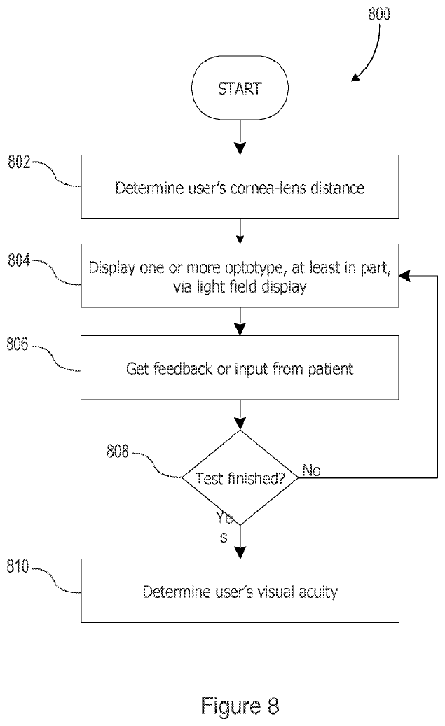

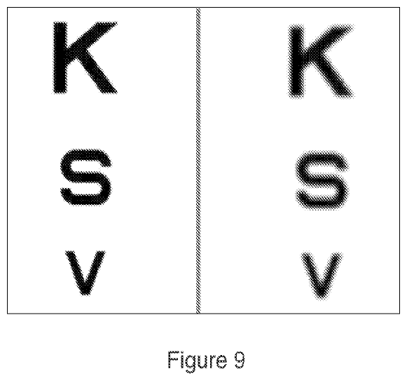

With reference to FIG. 8 and in accordance with one exemplary embodiment, a dynamic subjective vision testing method using vision testing system 100, generally referred to using the numeral 800, will now be described. As mentioned above, the use of a light field display enables refractor 102 to provide more dynamic and/or more modular vision tests than what is generally possible with traditional refractors/phoropters. Generally, method 800 seeks to diagnose a patient's reduced visual acuity and produce therefrom, in some embodiments, an eye prescription or similar.

In some embodiments, eye prescription information may include, for each eye, one or more of: distant spherical, cylindrical and/or axis values, and/or a near (spherical) addition value.

In some embodiments, the eye prescription information may also include the date of the eye exam and the name of the eye professional that performed the eye exam. In some embodiments, the eye prescription information may also comprise a set of vision correction parameter(s) for operating any vision correction light field displays using the systems and methods described below. In some embodiments, the eye prescription may be tied to a patient profile or similar, which may contain additional patient information such as a name, address or similar. The patient profile may also contain additional medical information about the user. All information or data (i.e. set of vision correction parameter(s), user profile data, etc.) may be kept on external database 124. Similarly, in some embodiments, the user's current vision correction parameter(s) may be actively stored and accessed from external database 124 operated within the context of a server-based vision correction subscription system or the like, and/or unlocked for local access via the client application post user authentication with the server-based system.

Refractor 102 being, in some embodiments, portable, a large range of environments may be chosen to deliver the vision test (home, eye practitioner's office, etc.). At the start, the patient's eye may be placed at the required location. This may be done by placing his/her head on a headrest or by placing the objective (i.e. eyepiece) on the eye to be diagnosed. As mentioned above, the vision test may be self-administered or partially self-administered by the patient. For example, the operator (e.g. eye professional or other) may have control over the type of test being delivered, and/or be the person who generates or helps generate therefrom an eye prescription, while the patient may enter inputs dynamically during the test (e.g. by choosing or selecting an optotype, etc.).

As will be discussed below, light field rendering methods described herein generally requires an accurate location of the patient's pupil center. Thus, at step 802, such a location is acquired. In some embodiments, such a pupil location may be acquired via eye tracker 110, either once, at intervals, or continuously. In other embodiments, the location may be derived from the device or system's dimension. For example, in some embodiments, the use a head-rest and/or an eye-piece or similar provides an indirect means of deriving the pupil location. In some embodiments, refractor 102 may be self-calibrating and not require any additional external configuration or manipulation from the patient or the practitioner before being operable to start a vision test.

At step 804, one or more optotypes is/are displayed to the patient, at one or more dioptric power (e.g. in sequence, side-by-side, or in a grid pattern/layout). The use of light field display 104 offers multiple possibilities regarding how the images/optotypes are presented, and at which dioptric power each may be rendered. The optotypes may be presented sequentially at different dioptric power, via one or more dioptric power increments. In some embodiments, the patient and/or operator may control the speed and size of the dioptric power increments.

In some embodiments, optotypes may also be presented, at least in part, simultaneously on the same image but rendered at a different dioptric power. For example, FIG. 9 shows an example of how different optotypes may be displayed to the patient but rendered with different dioptric powers simultaneously. These may be arranged in columns or in a table or similar. In FIG. 9, we see two columns of three optotypes (K. S. V), varying in size, as they are perceived by a patient, each column being rendered at different degrees of refractive correction (e.g. dioptric power). In this specific example, the optotypes on the right are being perceived as blurrier than the optotypes on the left. Ray tracing and adjusted image rendering techniques applicable to the generation of respective dioptric corrections for distinct portions of a rendered image (or for distinct optotypes or distinct renditions of a same optotype) are detailed in Applicant's above-reference U.S. Pat. No. 10,761,604.

Thus, at step 806, the patient would communicate/verbalise this information to the operator or input/select via, for example, control interface 126 the left column as the one being clearer. Thus, in some embodiments, method 800 may be configured to implement dynamic testing functions that dynamically adjust one or more displayed optotype's dioptric power in real-time in response to a designated input, herein shown by the arrow going back from step 808 to step 804 in the case where at step 808, the user or patient communicates that the perceived optotypes are still blurry or similar. In the case of sequentially presented optotypes, the patient may indicate when the optotypes shown are clearer. In some embodiments, the patient may control the sequence of optotypes shown (going back and forth as needed in dioptric power), and the speed and increment at which these are presented, until he/she identifies the clearest optotype. In some embodiments, the patient may indicate which optotype or which group of optotypes is the clearest by moving an indicator icon or similar within the displayed image.

In some embodiments, the optotypes may be presented via a video feed or similar.

In some embodiments, when using a reel of lenses or similar (for refractive components 112), discontinuous changes in dioptric power may be unavoidable. For example, the reel of lenses may be used to provide a larger increment in dioptric power, as discussed above. Thus, step 804 may in this case comprise first displaying larger increments of dioptric power by changing lens as needed, and when the clearest or less blurry optotypes are identified, fine-tuning with continuous or smaller increments in dioptric power using the light field display. In accordance with some embodiments, a LFSL position may be dynamically adjusted to provide larger increments in dioptric power, or again to migrate between different operative dioptric ranges, resulting in a smoother transition than would otherwise be observed when changing lenses in a reel. Similarly, the LFSL may be displaced in small increments or continuously for fine-tuning a displayed optotype. In the case of optotypes presented simultaneously, the refractive components 112 may act on all optotypes at the same time, and the change in dioptric power between them may be controlled by the light field display 104, for example in a static position to accommodate variations within a given dioptric range (e.g. applicable to all distinctly rendered optotypes), or across different LFSL positions dynamically selected to accommodate different operative ranges. In such embodiments, or, for example, when using an electrically tunable fluid lens or similar, the change in dioptric power may be continuous.

In some embodiments, eye images may be recorded during steps 802 to 806 and analyzed to provide further diagnostics. For example, eye images may be compared to a bank or database of proprietary eye exam images and analyzed, for example via an artificial intelligence (AI) or machine learning (ML) system, or similar. This analysis may be done by refractor 102 locally or via a remote server or database 124.

Once the correct dioptric power needed to correct for the patient's reduced visual acuity is defined at step 810, an eye prescription or vision correction parameter(s) may be derived from the total dioptric power used to display the best perceived optotypes.

In some embodiments, the patient, an optometrist or other eye-care professional may be able to transfer the patient's eye prescription directly and securely to his/her user profile store on said server or database 124. This may be done via a secure website, for example, so that the new prescription information is automatically uploaded to the secure user profile on remote database 124. In some embodiments, the eye prescription may be sent remotely to a lens specialist or similar to have prescription glasses prepared.

In some embodiments, vision testing system 100 may also or alternatively be used to simulate compensation for higher-order aberrations. Indeed, the light field rendering methods described above may be used to compensation for higher order aberrations (HOA), and thus be used to validate externally measured or tested HOA, in that a measured, estimated or predicted HOA can be dynamically compensated for using the system described herein and thus subjectively visually validated by the viewer in confirming whether the applied HOA correction satisfactory addresses otherwise experienced vision deficiencies.

As will be appreciated by the skilled artisan, different light field image processing techniques may be considered, such as those introduced above and taught by Pamplona and/or Huang, for example, which may also influence other light field parameters to achieve appropriate image correction, virtual image resolution, brightness and the like.

In accordance with various embodiments, various systems and methods described herein may provide an improvement over conventional light field displays through the provision or actuation of a translatable LFSL to, for instance, expand the dioptric range of perception adjustments accessible to a light field display system. For instance, and in accordance with some embodiments, a smartphone, tablet, television screen, embedded entertainment system (e.g. in a car, train, airplane, etc.) or the like, may benefit from a translatable LFSL to, for instance, increase a range of visual acuity corrections that may be readily accommodated with a similar degree of accommodation and rendering quality, or again, to expand a vision-based testing or accommodation range in a device so to effectively render optotypes or testing images across a greater perceptive adjustment, with, for instance, a given LFSL geometry and/or display resolution.

In accordance with at least one embodiment, a vision testing system, such as that described above, may comprise a translatable LFSL so to enable removal of one or more optical components (e.g. an electrically tunable lens, or one or more reels of lenses, or the like) from the light field system, while providing sufficient resolution to perform a vision-based assessment, non-limiting examples of which may include an eye exam or a cognitive impairment assessment. For instance, and in accordance with at least one embodiment, such a translatable LFSL may increase the widths of the peaks in FIGS. 6A to 6D, increasing the range of diopters that may be displayed with sufficient quality to perform an eye exam or cognitive assessment without requiring changing of optical components. Similarly, a translatable LFSL may further, in accordance with various embodiments, relate to reducing the size, weight, form factor, complexity, or the like, of a vision-based testing system. This may, for instance, reduce device cost while increasing the portability of a device for in-field assessments.

Various embodiments described hereafter may relate systems and methods related to vision testing systems that may employ a translatable LFSL. However, the skilled artisan will appreciate the scope and nature of the disclosure is not so limited, and that a dynamic or translatable LFSL may be applied in various other light field display systems and methods. Further, description will be provided with reference to mathematical equations and relationships that may be evaluated or considered in the provision of a light field system used to generate corrected content. Table 2 at the end of this section of the disclosure provides a summary of the terms and variables henceforth referred to for the reader's convenience.

The performance of a conventional eye exam may comprise placing a corrective lens in front of the eye being tested to correct the focal length of the eye having a depth DE, wherein the object may have a minimum feature separation of approximately 1 arcminute. To achieve this condition with, for instance, a light field-based visual testing system, the required retinal spot spacing is given by Equation 1.

$$Δ {y}_{ret}^{req} = 290 \times 1 {0}^{- 6} {D}_{E} \qquad ( Equation 1 )$$

As the depth of an unaberrated eye is typically assumed to be 25 mm, this results in a required spot spacing of approximately 7.27 μm, which, in accordance with some embodiments, increases by 1 mm per refractive error of −2.5 diopters.

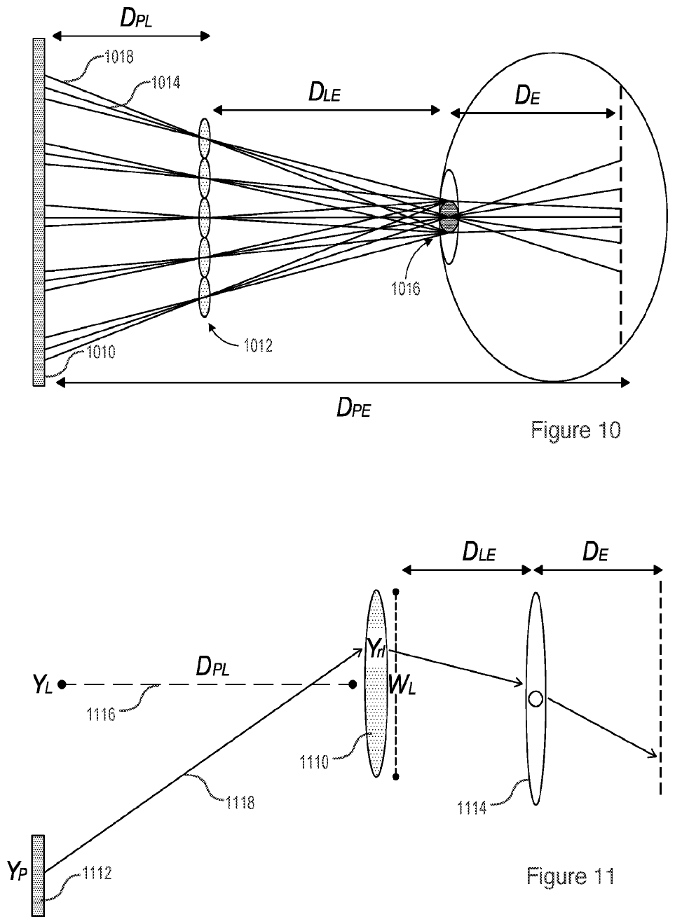

FIG. 10 schematically illustrates such an exemplary vision testing system 1000 comprising a pixelated display screen 1010 and MLA 1012, in accordance with at least one embodiment. In this non-limiting example, the nodal rays 1014 cross the centre of a pupil 1016, yielding a retinal nodal spot spacing of:

$$Δ {y}_{rr}^{N} = \frac{{D}_{e}}{{D}_{LE}} {P}_{L}$$

In this example, the spread around the nodal ray spot points is given in consideration of marginal rays 1018 reaching the edges of the pupil 1016:

$$Δ {y}_{rr}^{Δ N} = ( 1 - \frac{{D}_{e}}{{f}_{e}} + \frac{{D}_{e}}{{D}_{LE}} ) {W}_{ppl}$$

This corresponds to a width and number of pixels on the display 1010 equal to:

$$Δ {y}_{dis}^{Δ N} = \frac{{D}_{PL}}{{D}_{LE}} {W}_{ppl}$$

$${N}_{P}^{Δ N} = \frac{Δ {y}_{dis}^{Δ N}}{Δ {y}_{P}} = \frac{{D}_{PL}}{{D}_{LE}} \frac{{W}_{ppl}}{Δ {y}_{P}}$$

Accordingly, the beam spot spacing on the retina for one nodal band is given by:

$$Δ {y}_{ret} = \frac{Δ {y}_{rr}^{Δ N}}{{N}_{P}^{Δ N}} = ( 1 - \frac{{D}_{e}}{{f}_{e}} + \frac{{D}_{e}}{{D}_{L E}} ) \frac{{D}_{L E}}{{D}_{PL}} Δ {y}_{P}$$

In order to achieve a continuous spread of beam spots on a retina, the spread of each nodal band, in accordance with some embodiments, may be equal to or greater than the modal spacing (i.e. Δyᵣᵣ^{ΔN}≥yᵣᵣ^{N}), assuming uniform retinal spot spacing in a single nodal band of width Δyᵣᵣ^{ΔN}. For a number O of overlapping bands, to obtain uniform retinal sub-spacing, the central spot of each band may be shifted, in accordance with some embodiments, according to:

$$Δ {y}_{rr}^{N} \% Δ {y}_{ret} = \frac{Δ {y}_{r e t}}{ceil ( {O}_{ret} )}$$

$${O}_{ret} = \frac{Δ {y}_{rr}^{Δ N}}{Δ {y}_{r r}^{N}} = \frac{( 1 - \frac{{D}_{e}}{{f}_{e}} + \frac{{D}_{e}}{{D}_{L E}} ) {W}_{ppl}}{\frac{{D}_{e}}{{D}_{L E}} {P}_{L}} = [ \frac{{D}_{L E}}{{D}_{e}} - \frac{{D}_{LE}}{{f}_{e}} + 1 ] \frac{{W}_{ppl}}{{P}_{L}}$$

To avoid light from the one or more pixels hitting two points on the retina, the position of the MLA 1012 may be set, in accordance with some embodiments, to prevent overlap between different nodal bands on the display 1010. For nodal spacing on the display 1010 and a display spread of Δy_{dis}^{ΔN}:

$$Δ {y}_{dis}^{N} = \frac{{P}_{L}}{{D}_{LE}} ( {D}_{LE} + {D}_{PL} ) \geq Δ {y}_{dis}^{Δ N} = \frac{{D}_{PL}}{{D}_{LE}} {W}_{ppl} \qquad ( Equation 2 )$$

$${D}_{LE} \geq {D}_{PL} ( \frac{{W}_{ppl}}{{P}_{L}} - 1 )$$

The uniform retinal spacing of the overlapped nodal beams 1014 may therefore be given by the following (Equation 3), where the approximation denotes the possibility of a non-integer overlap factor O, in accordance with some embodiments:

$$Δ {y}_{Oret}^{uni} \approx \frac{Δ {y}_{ret}}{O} = \frac{( 1 - \frac{{D}_{e}}{{f}_{e}} + \frac{{D}_{e}}{{D}_{L E}} )}{[ \frac{{D}_{LE}}{{D}_{e}} - \frac{{D}_{LE}}{{f}_{e}} + 1 ] \frac{{W}_{ppi}}{{P}_{L}}} \frac{{D}_{L E}}{{D}_{PL}} Δ {y}_{P} \qquad ( Equation 3 )$$

To increase intensity delivered to the retina and increase contrast, the spread of light from the display pixels 1010 may, in accordance with some embodiments, be decreased as much as possible. This may be calculated, in some embodiments, in consideration of the distance between the display 1010, the pupil 1016, as well as the pitch and lenslet width of MLA 1012. The nodal ray 1014 from an extremum pixel to an extremum of the pupil 1016 on the other side of the optical axis may give one portion of the angular spread. Another portion of the angular spread may arise from the spread to fill the MLA 1012. Accordingly,

$${Δθ}_{Req}^{P} = \frac{Max ( \vert {y}_{p} \vert ) + \frac{{W}_{ppl}}{2}}{{D}_{PL} + {D}_{L E}} + \frac{{W}_{L}}{2 {D}_{PL}}$$

Assuming the resolving threshold of the eye for two retinal spots is ρ (i.e. the ratio of from maximum at which two beams cross), the required beam size may be calculated, in accordance with various embodiments. For instance, FIG. 11 schematically illustrates one lenslet 1110 where the pixel 1112 position is given by y_{P}. In FIG. 11, y_{L} is the lenslet central coordinate, W_{L} is the width of the lenslet 1110, and the eye 1114 is centered at the optical axis 1116 with the eye lens O centered at the origin. The ray 1118 propagation distance in this example is denoted as positive from left to right, while angles are denoted as positive in counter-clockwise direction, and y is positive above the optical axis 1116. Using a transfer matrix formulation of a thin lens, and in accordance with some embodiments, a ray 1118 that is emitted towards the lenslet 1110 is characterised by the following angle, where y_{RL} is the coordinate at which the ray 1118 hits the lenslet 1110:

$${θ}_{rp} = {tan}^{- 1} ( \frac{{y}_{rt} - {y}_{p}}{{D}_{PL}} )$$

Similarly, the ray angle exiting the lens may be given by, in accordance with some embodiments:

$${θ}_{rl} = - \frac{{y}_{rl} - {y}_{L}}{{f}_{L}} + {θ}_{rp}$$

In this exemplary embodiment, the position at the eye lens plane yᵣₑ and the angle θᵣₑ, after the eye lens O, may be given by, respectively:

$${y}_{re} = {y}_{rl} + {D}_{LE} {θ}_{rl}$$

$${y}_{re} = {y}_{ri} + {D}_{LE} ( {tan}^{- 1} ( \frac{{y}_{rl} - {y}_{p}}{{D}_{PL}} ) - \frac{{y}_{rl} - {y}_{L}}{{f}_{L}} , and$$

$${θ}_{re} = - \frac{{y}_{re}}{{f}_{ε}} + {θ}_{ri}$$

$${θ}_{re} = - \frac{{y}_{re}}{{f}_{ε}} - \frac{{y}_{rl} - {y}_{L}}{{f}_{L}} + {tan}^{- 1} ( \frac{{y}_{rl} - {y}_{P}}{{D}_{PL}} ) .$$

Further, ray separation on the eye lens O may be described, in accordance with some embodiments, by Equation 4:

$$( Equation 4 )$$

$$Δ {y}_{re}^{ab} = {y}_{rl}^{a} + {D}_{LE} ( {tan}^{- 1} ( \frac{{y}_{rl}^{a} - {y}_{p}^{a}}{{D}_{PL}} ) - \frac{{y}_{rl}^{a} - {y}_{L}^{a}}{{f}_{L}} ) -$$

$$[ {y}_{rl}^{b} + {D}_{LE} ( {tan}^{- 1} ( \frac{{y}_{rl}^{b} - {y}_{P}^{b}}{{D}_{PL}} ) - \frac{{y}_{rl}^{b} - {y}_{L}^{b}}{{f}_{L}} ) ]$$

$$Δ {θ}_{re}^{ab} = \text{} - \frac{Δ {y}_{re}^{ab}}{{f}_{E}} + [ \text{} {tan}^{- 1} ( \frac{{y}_{rl}^{a} - {y}_{P}^{a}}{{D}_{PL}} ) - \frac{{y}_{rl}^{a} - {y}_{L}^{a}}{{f}_{L}} ] -$$

$$[ {tan}^{- 1} ( \frac{{y}_{rl}^{b} - {y}_{P}^{b}}{{D}_{PL}} ) - \frac{{y}_{rl}^{b} - {y}_{L}^{b}}{{f}_{L}} ]$$

In accordance with some embodiments, the spot size from one lenslet 1010 (i.e. y_{L}^{a}=y_{L}^{b}) on the eye lens O may be found by tracing marginal rays Mr (e.g. marginal rays 1018 in FIG. 10):

$$Δ {y}_{Mre}^{ab} = {W}_{L} + {D}_{LE} ( {tan}^{- 1} ( \frac{{D}_{PL} {W}_{L}}{{D}_{PL}^{2} + {( {y}_{L} - {y}_{p} )}^{2} - {( \frac{{w}_{L}}{2} )}^{2}} ) - \frac{{W}_{L}}{{f}_{L}} ) = {W}_{L} ( 1 - \frac{{D}_{LE}}{{f}_{L}} ) + {D}_{LE} {tan}^{- 1} ( \frac{{D}_{PL} {W}_{L}}{{D}_{PL}^{2} + {( {y}_{L} - {y}_{p} )}^{2} - {( \frac{{W}_{L}}{2} )}^{2}} ) \approx {W}_{L} ( 1 - \frac{{D}_{LE}}{{f}_{L}} ) + {D}_{LE} \frac{{W}_{L}}{{D}_{PL}} , and$$

$${Δθ}_{Mre}^{ab} = - \frac{Δ {y}_{Mre}^{ab}}{{f}_{E}} - \frac{{W}_{L}}{{f}_{L}} + {tan}^{- 1} ( \frac{{D}_{PL} {W}_{L}}{{D}_{PL}^{2} + {( {y}_{L} - {y}_{p} )}^{2} - {( \frac{{W}_{L}}{2} )}^{2}} ) \approx - \frac{Δ {y}_{Mre}^{ab}}{{f}_{E}} - \frac{{W}_{L}}{{f}_{L}} + \frac{{W}_{L}}{{D}_{PL}}$$

Further, pupil size Wₚₚₗ may be accounted for, in accordance with some embodiments as Equation 5:

$${Δθ}_{re} = \{ \begin{matrix}{Δθ}_{Mre}^{ab} & Δ {y}_{Mre}^{ab} \leq {W}_{ppl} \\ {Δθ}_{Mre}^{ab} \cdot \frac{{W}_{ppl}}{\vert Δ {y}_{Mre}^{ab} \vert} & \vert Δ {y}_{Mre}^{ab} \vert > {W}_{ppl}\end{matrix} \qquad ( Equation 5 )$$

$$Δ {y}_{re} = \frac{Δ {y}_{Mre}^{ab}}{\vert Δ {y}_{Mre}^{ab} \vert} \cdot Max ( {W}_{Ppl} , \vert Δ {y}_{Mre}^{ab} \vert )$$

where the following identity was used:

$${tan}^{- 1} ( \frac{{y}_{rl}^{a} - {y}_{P}}{{D}_{PL}} ) - {tan}^{- 1} ( \frac{{y}_{rl}^{b} - {y}_{P}}{{D}_{PL}} ) = {tan}^{- 1} ( \frac{\frac{{y}_{rl}^{a} - {y}_{P} - {y}_{rl}^{b} + {y}_{P}}{{D}_{PL}}}{1 + \frac{{y}_{rl}^{a} - {y}_{P}}{{D}_{PL}} \cdot \frac{{y}_{rl}^{b} - {y}_{P}}{{D}_{PL}}} ) = {tan}^{- 1} ( \frac{{D}_{PL} ( {y}_{rl}^{a} - {y}_{rl}^{b} )}{{D}_{PL}^{2} + ( {y}_{rl}^{a} - {y}_{p} ) ( {y}_{ri}^{b} - {y}_{P} )} )$$

For ray position and angles at the retina:

$${θ}_{rr} = {θ}_{re}$$

$${y}_{rr} = {y}_{re} + {D}_{E} {θ}_{re} ,$$

the spot size and divergence of beams are therefore given, in accordance with various embodiments, by:

$$Δ {y}_{rr} = Δ {y}_{re} + {D}_{E} {Δθ}_{re}$$

$${Δθ}_{rr} = Δ {θ}_{re} .$$

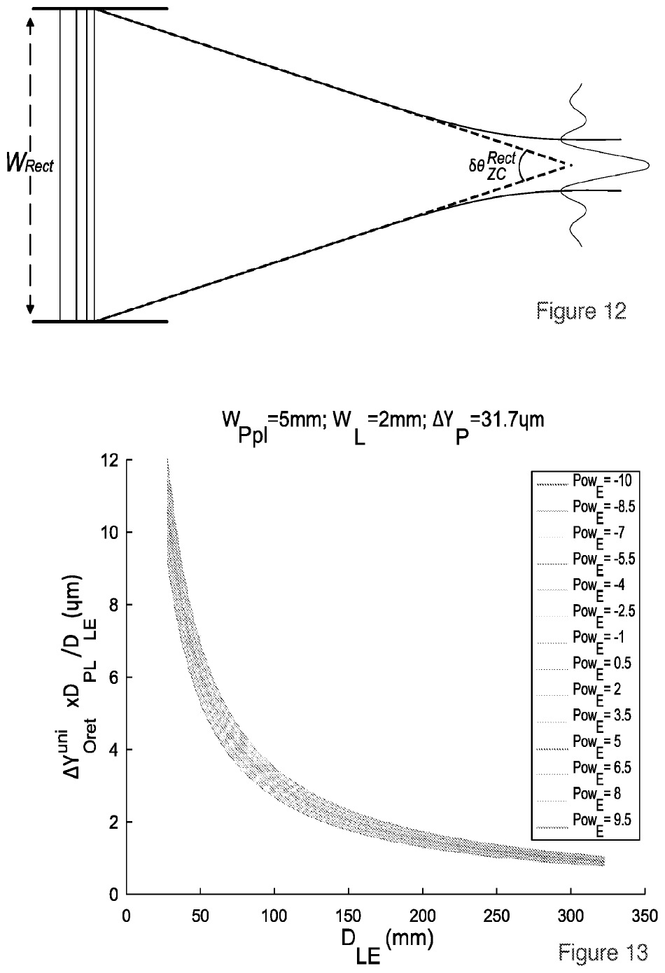

In accordance with various embodiments, various light field systems and methods may comprise the treatment of rays as a rectangular beam. One such exemplary embodiment is shown in FIG. 12, where focusing of a rectangular beam is schematically illustrated. With a ray tracing model, a beam size may approach a minimum theoretical size of zero width. In a practical system, however, a diffraction model may be employed. At the plane where the beam size is determined to be zero, the divergence of the beam may be used to obtain the actual size of the beam. Accordingly, assuming a beam with a rectangular width of W_{Rect}:

$$E = rect ( \frac{y}{{W}_{Rect}} )$$

diffracted light is therefore given by:

$${E}_{D} = Sin c ( \frac{y}{λ z} {W}_{Rect} ) ;$$

$$Sin c ( y ) = {lim}_{u \to x} \frac{Sin ( π u )}{π u}$$

To calculate beam divergence, in accordance with some embodiments, the first zero crossing width (W_{D}^{ZC}) of the diffracted beam Sinc(W_{Rect} y/λz) may be considered, which occurs at:

$$\frac{Δ y}{λ z} {W}_{Rect} = 2$$

$${W}_{D}^{ZC} = Δ y = 2 \frac{λ z}{{W}_{Rect}}$$

The divergence of the diffracted beam is therefore, and in accordance with some embodiments:

$$δ {θ}_{ZC}^{Rect} = 2 {tan}^{- 1} \frac{{W}_{Rect} / 2}{z} \to tan ( \frac{δ {θ}_{ZC}^{Rect}}{2} ) = \frac{{W}_{Rect}}{2 z}$$

$${W}_{D}^{ZC} = \frac{λ}{tan ( \frac{δ {θ}_{ZC}^{Rect}}{2} )}$$

Hence, the beam spot size on the retina W_{Mrr} may be given by Equation 6:

$${W}_{Mrr} = \frac{\vert Δ {y}_{rr} \vert}{Δ {y}_{rr}} \cdot max ( \vert Δ {y}_{rr} \vert , \vert \frac{λ}{tan ( \frac{δ {θ}_{ZC}^{Rect}}{2} )} \vert ) \qquad ( Equation 6 )$$

In accordance with various embodiments, a beam may be converging or diverging and/or collimated. For instance, considering adjacent lenslets, i.e. (y_{L}^{a}−y_{L}^{b}=P_{L}).

$$Δ {y}_{re}^{ab} = {y}_{rl}^{a} - {y}_{rl}^{b} + {D}_{LE} ( {tan}^{- 1} ( \frac{{y}_{rl}^{a} - {y}_{P}}{{D}_{PL}} ) - {tan}^{- 1} ( \frac{{y}_{rl}^{b} - {y}_{P}}{{D}_{PL}} ) + \frac{{y}_{rl}^{b} - {y}_{rl}^{a} + {P}_{L}}{{f}_{L}} ) \approx {y}_{rl}^{a} - {y}_{rl}^{b} + {D}_{LE} ( \frac{{y}_{rl}^{a} - {y}_{rl}^{b}}{{D}_{PL}} + \frac{{y}_{rl}^{b} - {y}_{rl}^{a} + {P}_{L}}{{f}_{L}} )$$

In the exemplary embodiment of D_{PL} being less than or equal to f_{L} in diverging or collimated beams, as beams are expanding, marginal rays at adjacent edges of the lenslets may be prevented from overlapping at the pupil (yᵣₗ^{a}−yᵣₗ^{b}=P_{L}−W_{L}):

$$Δ {y}_{re}^{ab} \approx {P}_{L} - {W}_{L} + {D}_{LE} ( \frac{{P}_{L} - {W}_{L}}{{D}_{PL}} + \frac{2 {P}_{L} - {W}_{L}}{{f}_{L}} ) \geq {W}_{ppl} ,$$

$$for$$

$${P}_{L} = {W}_{L} : {D}_{LE} \geq \frac{{f}_{L}}{{W}_{L}} {W}_{ppl} .$$

For a converging beam, and in accordance with some embodiments, a limit may be set on viewer distance where rays from further adjacent lenslet edges

$$( {y}_{rl}^{a} - {y}_{rl}^{b} = {W}_{L} + {P}_{L} ; {y}_{rl}^{a} = {y}_{L} + \frac{{W}_{L}}{2} ; {y}_{rl}^{b} = {y}_{L} - \frac{{W}_{L}}{2} - {P}_{L} )$$

may overlap on the pupil:

$$Δ {y}_{re}^{ab} \approx {W}_{L} + {P}_{L} + {D}_{LE} ( \frac{{W}_{L} + {P}_{L}}{{D}_{PL}} - \frac{{W}_{L}}{{f}_{L}} ) \geq {W}_{ppl}$$

For P_{L}=W_{L}:

$$2 {W}_{L} + {D}_{LE} {W}_{L} ( \frac{2}{{D}_{PL}} - \frac{1}{{f}_{L}} ) \geq {W}_{ppl}$$

Accordingly, for P_{L}=W_{L}:

$$\{ \begin{matrix}\frac{{D}_{LE}}{{D}_{PL}} \geq \frac{{W}_{ppl}}{{W}_{L}} \frac{{f}_{L}}{{D}_{PL}} & ; {D}_{PL} \leq {f}_{L} \\ \frac{{D}_{LE}}{{D}_{PL}} > \frac{{W}_{ppl} - 2 {W}_{L}}{{W}_{L} ( 2 - \frac{{D}_{PL}}{{f}_{L}} )} & ; {f}_{L} < {D}_{PL} < 2 {f}_{L} \\ \frac{{D}_{LE}}{{D}_{PL}} < \frac{{W}_{ppl} - 2 {W}_{L}}{{W}_{L} ( 2 - \frac{{D}_{PL}}{{f}_{L}} )} & ; {D}_{PL} > 2 {f}_{L}\end{matrix} \qquad ( Equation 7 )$$

Further, from Equation 2 above,

$$\frac{{D}_{LE}}{{D}_{PL}} \geq ( \frac{{W}_{ppl}}{{W}_{L}} - 1 ) .$$

While the above described conditions, in accordance with some embodiments, may not be strong if, for instance, part of the interference beam hits the pupil, it may be difficult to notice, particularly at reduced intensities (D_{PL} not equal to f_{L}), due to the inverse square law. Accordingly, the conditions arising from Equation 2 may have more significant importance.

Further, and in accordance with other embodiments, angular pitch on a pupil for a corrected eye lens may be obtained by the following, which may be more similar to a conventional eye exam:

$${θ}_{rl} = - \frac{{y}_{rl} - {y}_{L}}{{f}_{L}} + {tan}^{- 1} ( \frac{{y}_{rl} - {y}_{P}}{{D}_{PL}} )$$

$$Δ {θ}_{rl} = [ {tan}^{- 1} ( \frac{{y}_{rl} - {y}_{P}}{{D}_{PL}} ) - {tan}^{- 1} ( \frac{{y}_{rl} - ( {y}_{P} + Δ {y}_{P} )}{{D}_{PL}} ) ] = {tan}^{- 1} ( \frac{{D}_{PL} Δ {y}_{P}}{{D}_{PL}^{2} + {[ {y}_{rl} - {y}_{P} ]}^{2} - Δ {y}_{P} [ {y}_{rl} - {y}_{P} ]} ) \approx \frac{Δ {y}_{P}}{{D}_{PL}}$$

In this example, the separation between the rays from adjacent pixels on the eye lens may be obtained using Equation 5 above, with yᵣₗ^{a}=yᵣₗ^{b} and y_{L}^{a}=y_{L}^{b}.

$$Δ {y}_{re}^{Res} = {D}_{LE} [ {tan}^{- 1} ( \frac{{y}_{rl}^{a} - {y}_{P} + Δ {y}_{P}}{{D}_{PL}} ) - {tan}^{- 1} ( \frac{{y}_{rl}^{b} - {y}_{P}}{{D}_{PL}} ) ]$$

$$Δ {y}_{re}^{Res} = {D}_{LE} {tan}^{- 1} ( \frac{Δ {y}_{P} {D}_{PL}}{{D}_{PL}^{2} + ( {y}_{rl}^{a} - {y}_{P} + Δ {y}_{P} ) ( {y}_{rl}^{a} - {y}_{P} )} ) \approx {D}_{LE} \frac{Δ {y}_{P}}{{D}_{PL}}$$

After the eye lens, the angular pitch of the rays in a single nodal band (e.g. that in FIG. 10) may be found as:

$$Δ {θ}_{re}^{Res} = \frac{Δ {y}_{re}^{ab}}{{f}_{E}} + {tan}^{- 1} ( \frac{Δ {y}_{P} {D}_{PL}}{\begin{matrix}{D}_{PL}^{2} + ( {y}_{rl}^{a} - {y}_{P} + Δ {y}_{P} ) \\ ( {y}_{rl}^{a} - {y}_{P} )\end{matrix}} ) \approx - \frac{{D}_{LE}}{{D}_{PL}} \frac{Δ {y}_{P}}{{f}_{E}} + \frac{Δ {y}_{P}}{{D}_{PL}} = \frac{Δ {y}_{P}}{{D}_{PL}} [ 1 - \frac{{D}_{LE}}{{f}_{E}} ]$$

$$Δ {y}_{ret} = {D}_{LE} \frac{Δ {y}_{P}}{{D}_{PL}} + [ 1 - \frac{{D}_{LE}}{{f}_{E}} ] \frac{Δ {y}_{P}}{{D}_{PL}} {D}_{E} = [ ( 1 - \frac{{D}_{e}}{{f}_{e}} ) + \frac{{D}_{e}}{{D}_{LE}} ] \frac{{D}_{LE}}{{D}_{PL}} Δ {y}_{P} = \frac{Δ {y}_{rr}^{Δ N}}{{N}_{P}}$$

In accordance with some embodiments, a light field angular view (FoV) may be given by the distance from the display to the eye, and the MLA position. Assuming there are sufficient lenslets to support the display size of a light field system, the FoV and spread on the retina may therefore, in accordance with some embodiments, by given by, respectively:

$$( Equation 8 )$$

$$FoV = ( \frac{Floor ( \frac{{D}_{LE}}{{D}_{LE} + {D}_{PL}} \frac{{W}_{Disp}}{{P}_{L}} ) {P}_{L}}{{D}_{LE}} ) + max ( \frac{{W}_{ppl}}{{D}_{LE}} - \frac{{W}_{ppl}}{{f}_{E}} , 0 )$$

$$and$$

$${Sprd}_{ret} = FoV \cdot {D}_{E} .$$

Having established various relationships between parameters accessible to various light field systems and methods, various exemplary embodiments will now be described with reference to Equations 1 to 7 above and to FIGS. 13 to 17D.

In accordance with at least one embodiment, the uniform spacing of retinal beam spots for overlapping nodal bands are plotted in FIG. 13 as a function of D_{LE} and F_{E}, normalised to D_{PL}/D_{LE} using Equation 3 above. In this example, spacing is dependent on the eye focal length. Using Δyᵣₑₜ^{req}=7.27 μm and choosing D_{LE}>150 mm allows for spacing of less than 3 μm×D_{LE}/D_{PL}. Accordingly, D_{LE}/D_{PL} may be greater than 2.6.

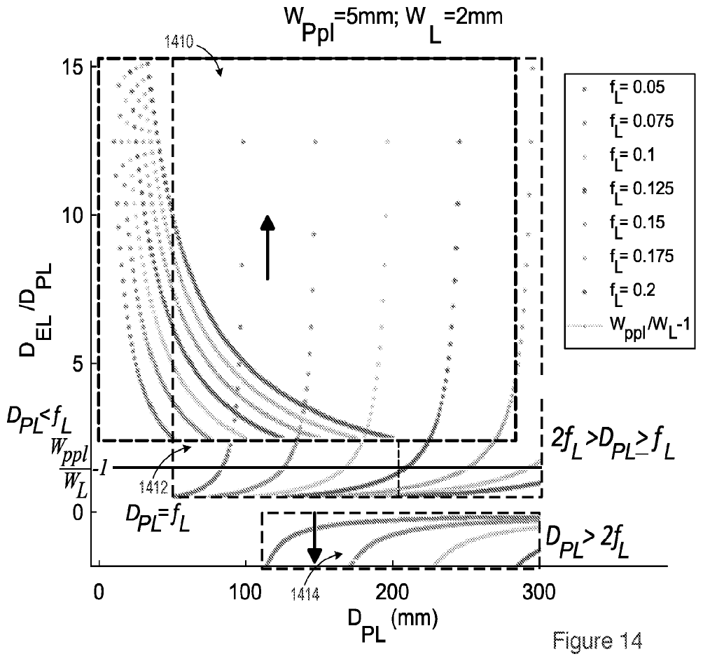

FIG. 14 shows an exemplary plot of the different regions of D_{LE}/D_{PL}, where arrows indicate the direction that satisfies the condition of Equation 7 above. The (in some embodiments, stronger) condition of Equation 2 may require that D_{LE}/D_{PL}>1.5. To have a reasonable form factor, and in accordance with some embodiments, the regions 1410 and 1412 of FIG. 14, and/or those in which D_{PL} is less than or equal to F_{L}, may be preferred for embodiments of a light field system or method. Conversely, region 1414 of FIG. 14 may comprise one that may not be satisfied, as beyond this range, a system form factor may rapidly escalate to one that is unreasonable or undesirable.

In accordance with at least one embodiment, a light field system may comprise values of D_{PL}=100 mm and D_{LE}=250 mm, with f_{L}=100 mm.

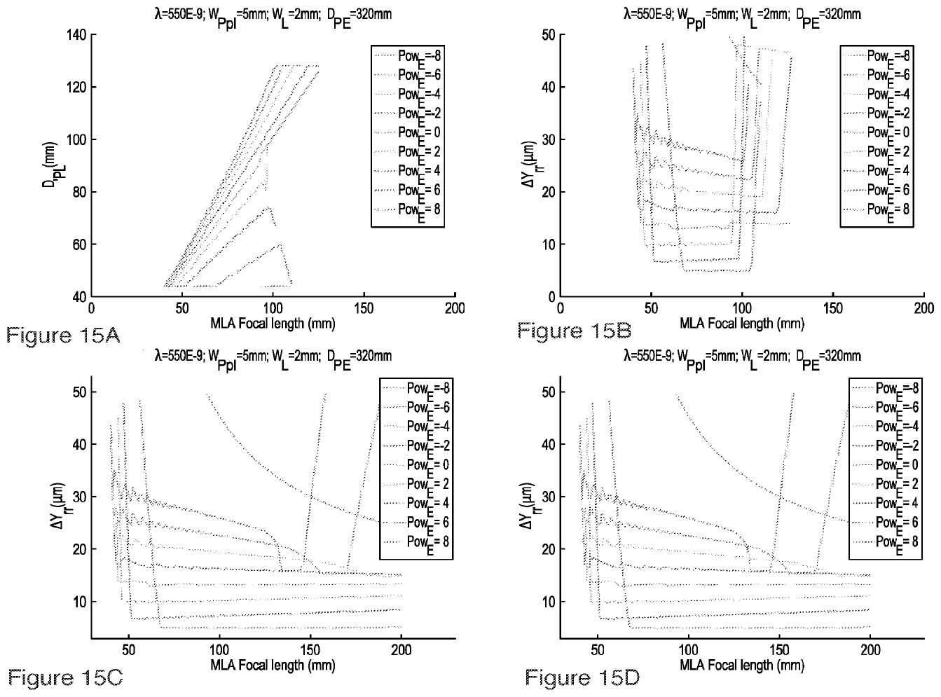

FIGS. 15A to 15D are illustrative plots for a system having a display-to-eye distance D_{PE} of 320 mm. In these exemplary embodiments, the calculated minimum beam size is a function of the MLA focal length and display-to-MLA distance. FIGS. 15A and 15B are illustrative plots showing calculations performed with the condition of avoiding single pixel beam interference from different lenslets. On the other hand, FIGS. 15C and 15D show the calculation without this condition applied.

Such calculations, in accordance with some embodiments, may be used to evaluate, for instance, potential device specifications, rather than evaluating actual retinal beam size values. For example, the actual spot size in an eye that is complex in nature may differ from that determined when using a simple lens model. Accordingly, and in accordance with different embodiments, the actual retinal spot size applied relative to the retinal spot spacing may be dependent on an eye functionality and/or empirical data, and may be calibrated experimentally.

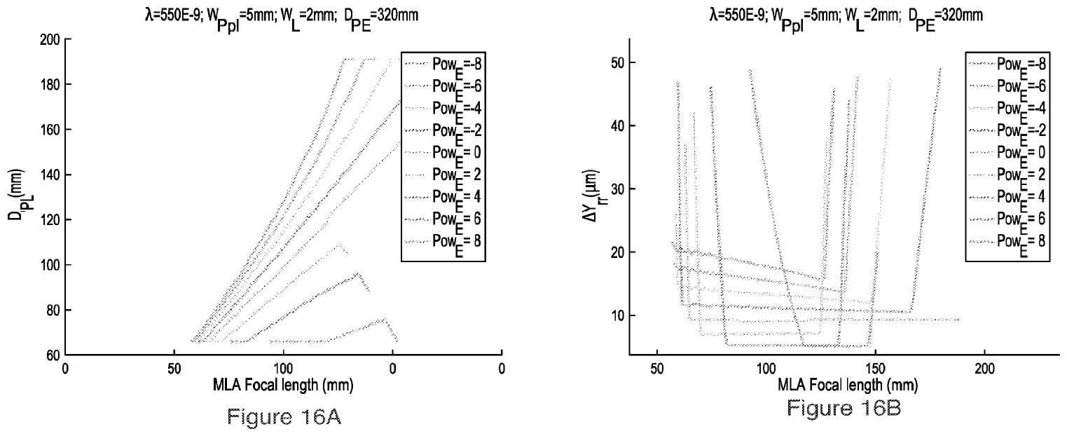

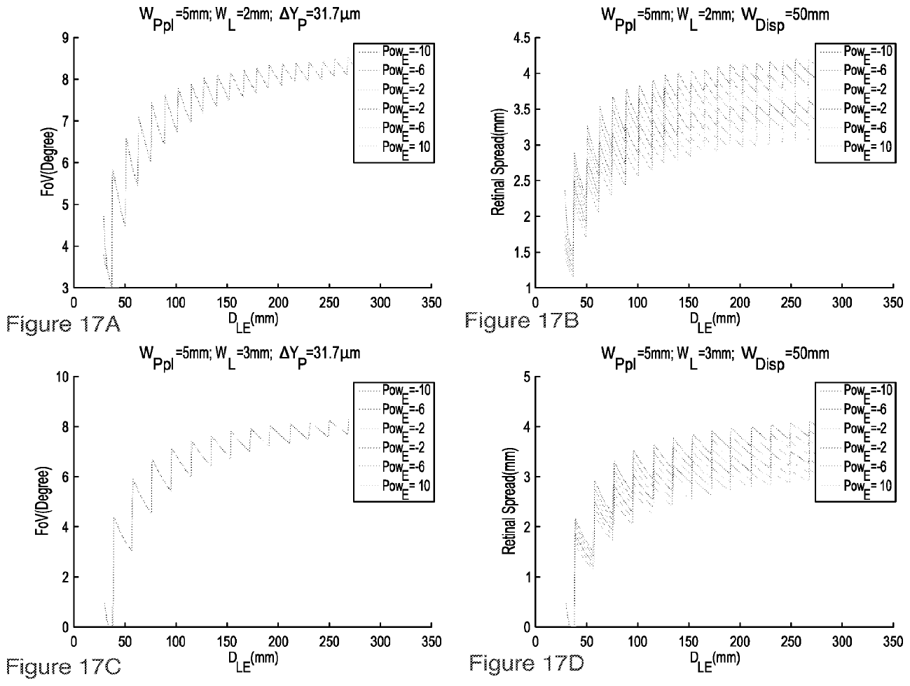

Further, and in accordance with various embodiments, increasing the lenslet size may contribute to minimising the retinal spot. This is shown, by way of example, only, as the non-limiting illustrative plots of FIGS. 16A and 16B.

In accordance with various embodiments, FIGS. 17A to 17D show exemplary plots of the FoV and retina spread for a display-to-eye distance of 320 mm. In these examples, and in accordance with various embodiments, larger lenslets result in smaller FoV and retinal spread. Further, retinal spread may be reduced for positive refractive errors, an effect that may be considered when calculating a light field to be displayed, in accordance with various embodiments.

In accordance with at least one embodiment, Table 1 below shows exemplary dioptric error corrections obtained in practice for various D_{PL} distances in millimetres. In this non-limiting example, the light field display system comprised an MLA with a lenslet focal length of 106 mm, with 2 mm pitch and lenslet width, a 31.7 μm pixel size display, and an eye-to-display distance of 320 mm.

TABLE 1
Dioptric Error vs D_{PL}

| | Diopter Error | | | | | | |
|---|---|---|---|---|---|---|---|
| | −8.25 | −7.25 | −6.25 | −5.25 | −4.25 | −3.25 | −2.25 |
| D_{PL} (mm) | 120 | 110 mm | 100 | 99 | 85 | 73 | 53 |

In the example of Table 1, while measured values did not exactly match calculated values, the general trend expected based on Equations 1 to 7 were observed, in accordance with various embodiments. That is, while the actual eye is more complex than the simple lens assumed in calculations, such deviation is expected, and calibration may be performed, for instance, based on eye functionality statistical data or the like, in accordance with some embodiments.

Nevertheless, the results of Table 1 highlight, in accordance with some embodiments, how a dioptric error range may be enhanced over that of conventional systems through, for instance, a dynamic displacement of a LFSL relative to a pixelated display. For instance, while conventional systems may maintain a fixed MLA at a distance from the display corresponding to the focal length of the lenslets, placing the LFSL at a distance other than that corresponding to the lenslet focal length from the display, or dynamically displacing it relative thereto, may increase the dioptric range of perception adjustments accessible to the light field display system.

In yet other embodiments, both the pixelated display and the LFSL may remain in place while still achieving a similar effect by otherwise adjusting an optical path length between them. Indeed, the selective introduction of one or more liquid cells or transparent (e.g. glass) blocks within this optical path may provide a similar effect. Other means of dynamically adjusting an optical path length between the pixelated display and LFSL may also be considered without limitation, and without departing from the general scope and nature of the present disclosure.

In any such embodiment, such a LFSL (i.e. one that may be disposed at a selectively variable optical path length from a display screen), herein also referred to as a dynamic LFSL, may comprise various light field shaping layers known in the art, such as an MLA, a parallax barrier, an array of apertures, such as a pinhole array, or the like, and may be fabricated by various means known in the art.

In accordance with various embodiments, a dynamic LFSL may be coupled with a display screen via, for instance, one or more actuators that may move the LFSL towards or away from (i.e. perpendicularly to) a digital display, and thus control, for instance, a range of dioptric corrections that may be generated by the digital display. Naturally, an actuator may otherwise dynamically displace a pixelated display relative to a fixed LFSL to achieve a similar effect in dynamically adjusting an optical path length between them.

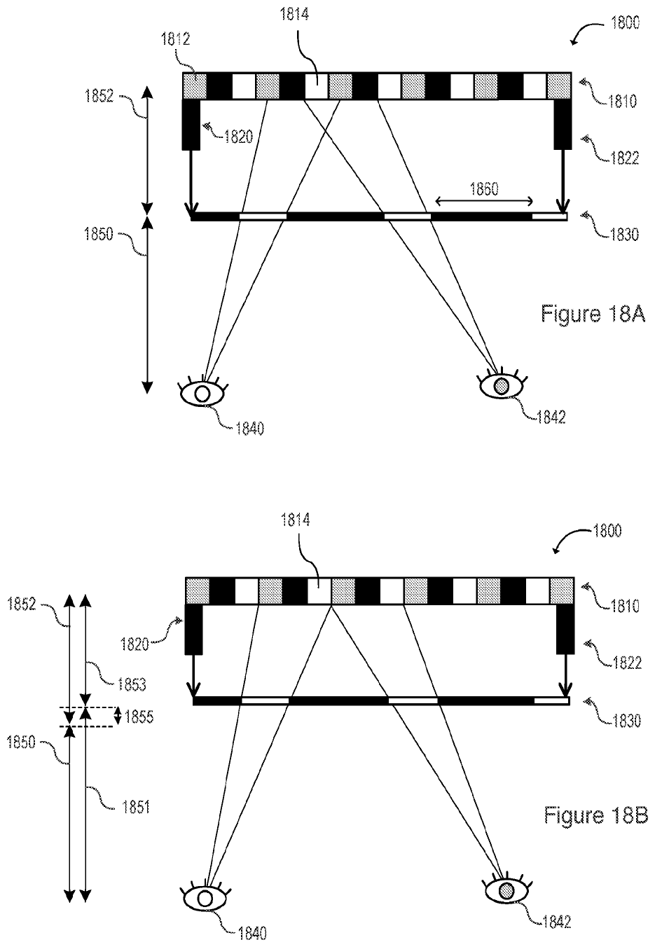

For instance, FIG. 18A shows a schematic of a display system 1800 (not to scale) comprising a digital display 1810 having an array of pixels 1812. In this example, conventional red, green, and blue subpixels are shown as grey, black, and white pixels, respectively. In order to generate a light field, a LFSL 1830, represented in FIG. 18A as a parallax barrier 1830 having a barrier width (pitch) 1860, is coupled to the display 1810 via actuators 1820 and 1822, and is disposed between the display 1810 and two viewing locations 1840 and 1842, represented by white and grey eyes, respectively.

In accordance with various embodiments, view zones 1840 and 1842 may correspond to, for instance, two different eyes of a user, or eyes of two or more different users. While various light field shaping systems may, in accordance with various embodiments, address more than one eye, for instance to provide two different dioptric corrections to different eyes of the user, this embodiment will henceforth be described with reference to the first eye 1840, for simplicity. Further, it will be appreciated that while the dynamic LFSL 1830 in FIG. 18A is shown in one dimension, various other embodiments relate to the LFSL 1830 comprising a two dimensional configuration, such as a lenslet array or a static or dynamic 2D parallax barrier (e.g. an LCD screen that may be activated to provide an arbitrary configuration of opaque and transparent pixels), and/or may comprise a MLA 1830 that may be dynamically adjusted or placed at various distances from the display 1810 to provide a designated light field to, for instance, enable a designated range of dioptric corrections via activation of appropriate pixels 1812.

FIG. 18A shows a first configuration in which pixels of the display 1810 are activated to provide, for instance, a perception adjustment corresponding to a designated dioptric error correction (e.g. a dioptric error of −4.25), for a viewer at the first viewing location 1840. In this example, the user is at a distance 1850 from the dynamic LFSL 1830, while the LFSL 1830 is at a distance 1852 from the screen 1810, where the distance 1852 corresponds to, for instance, the focal length of the microlens LFSL 1830, as may be employed in conventional light field systems. Without adjustment, the user, at a distance corresponding to the sum of distances 1850 and 1852 (e.g. the distance between a display screen and the user's eye in a refractor system used for displaying various optotypes), is unable to perceive, at a sufficiently high rendering quality, the dioptric adjustment (e.g. −4.25 diopters) provided by the activated pixels, for instance due to geometrical constraints and/or limitations in screen resolution. For instance, the configurational parameters may lead to an undesirable viewing experience, and/or a lack of perception of the visual correction requiring a particular configuration of activated pixels as a result of the considerations noted above. For example, while a perceptive adjustment may be rendered, it may not exhibit the level of resolution and optical discernment required to conduct an effective vision test.

In accordance with various embodiments, actuators 1820 and 1822 may translate the dynamic LFSL 1830 towards or away from the display 1810, i.e. at a distance within or beyond the MLA focal length for a lenslet LFSL, to dynamically improve a perception adjustment rendering quality consistent with testing requirements and designated or calibrated in accordance with the corrective range of interest. In FIG. 18B, actuators 1820 and 1822 have reconfigured the display system 1800 such that the LFSL 1830 has been dynamically shifted towards the display 1810 by a distance 1855, resulting in a new distance 1851 between the LFSL 1830 and viewing location 1840, and a new separation 1853 between the display 1810 and LFSL 1830. In this more optimised configuration, perception adjustment quality can be improved to a desired, designated or optimized level thereby allowing for a given optical effect or test to be adequately or optimally conducted. The skilled artisan will appreciate that this description is for illustrative purposes, only, and that various dynamic LFSLs, such as a dynamic MLA, may function within this scope of the disclosure in accordance with various mechanisms to provide, for instance, a larger or designated range of image perception adjustments.

The skilled artisan will appreciate that various actuators may be employed to dynamically adjust a LFSL with, for instance, high precision, while having a robustness to reliably adjust a LFSL or system thereof (e.g. a plurality of LFSLs, a LFSL comprising a plurality of PBs, MLAs, or the like). Furthermore, embodiments comprising heavier LFSL substrates (e.g. Gorilla glass or like tempered glass) may employ, in accordance with some embodiments, particularly durable and/or robust actuators, examples of which may include, but are not limited to, electronically controlled linear actuators, servo and/or stepper motors, rod actuators such as the PQ12, L12, L16, or P16 Series from Actuonix® Motion Devices Inc., or the like. The skilled artisan will further appreciate that an actuator or actuator step size may be selected based on a screen or lenslet size, whereby larger elements may, in accordance with various embodiments, require only larger steps to introduce distinguishable changes in user perception of various pixel configurations. Further, various embodiments relate to actuators that may communicate with a processor/controller via a driver board, or be directly integrated into a processing unit for plug-and-play functionality.

While FIGS. 18A and 18B show a dynamic adjustment of a LFSL layer in a direction perpendicular to the screen, the skilled artisan will appreciate that such perpendicular adjustments (i.e. changing the separation 1853 between the display 1810 and LFSL 1830) may result in a modification of an optimal viewing distance 1851 from the LFLS 1830. As such, the separation 1853 may be adjusted to configure a system 1800 for a wide range of preferred viewing positions in addition to, or alternatively to, providing a range of or designated perception adjustments.

Furthermore, as readily available actuators may finely adjust and/or displace the LFSL 1830 with a high degree of precision (e.g. micron-precision), various embodiments of a dynamic LFSL as herein described relate to one that may be translated perpendicularly to a digital display to enhance user experience.

In accordance with various embodiments, a translatable LFSL, such as that of FIGS. 18A and 18B, may be employed in a vision-based testing platform. For instance, and as discussed above, a refractor or phoropter for assessing a user's visual acuity may comprise a translatable LFSL operable to be placed at, for instance, a designated distance from a display screen, so to provide a system geometry that enables display of a preferred perception adjustment (e.g. dioptric correction). For instance, a conventional light field refractor having a LFSL (e.g. MLA) disposed at a fixed distance (e.g. a focal length away) from a pixelated display screen may have inherent limitations arising from, for instance, screen resolution or size. Such systems may therefore only enable display of a particular range of optotypes without the use of additional optical components. However, and in accordance with various embodiments, a light field refractor having a translatable LFSL may allow for adjustment of the system geometry, thereby enabling an adjusted range of optotypes that may be displayed, without the use of one or more of the optical elements required by the conventional system.

Similarly, and in accordance with other embodiments, a light field display system having a translatable LFSL may be employed to perform cognitive impairment tests, as described above. In some embodiments, the use of a light field display in performing such assessments may allow for accommodation of a reduced visual acuity of a user undergoing a test. For instance, a dioptric correction may be applied to displayed content to provide the user with an improved perception thereof, and therefore improve the quality of evaluation. Further, such a system may employ a translatable or dynamic LFSL to quickly and easily apply the appropriate dioptric correction for the user. In some embodiments, such a light field system may be portable (e.g. to quickly assess an athlete's cognitive ability following a collision during performance of a sport), and may therefore benefit from the removal of one or more optical components required to generate an adequate range of optotypes for testing.

A LFSL as herein disclosed, in accordance with various embodiments, may further or alternatively be dynamically adjusted in more than one direction. For instance, in addition to providing control of the distance between a display and a single LFSL (e.g. a single parallax barrier) oriented substantially parallel thereto, the LFSL may further be dynamically adjustable in up to three dimensions. The skilled artisan will appreciate that actuators, such as those described above, may be coupled to displace any one LFSL, or system comprising a plurality of light field shaping components, in one or more directions. Yet further embodiments may comprise one or more LFSLs that dynamically rotate in a plane of the display to, for instance, change an orientation of light field shaping elements relative to a pixel or subpixel configuration. Furthermore, in addition to providing control over the distance between a LFSL and a screen, a LFSL as herein described may further allow for dynamic control of a LFSL layer pitch, or barrier width, in embodiments comprising a parallax barrier. In accordance with various further embodiments, a light field shaping system or device may comprise a plurality of independently addressable parallax barriers. These and similar embodiments are further described in the above-referenced U.S. Patent Application No. 63/056,188.

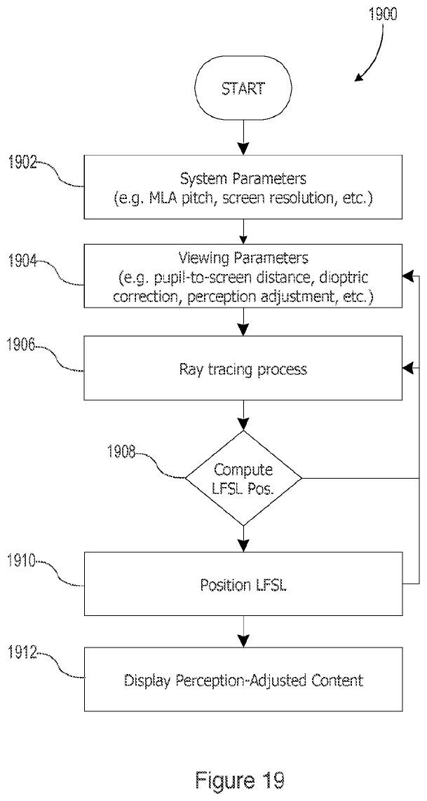

In accordance with at least one embodiment, FIG. 19 is a diagram illustrating an exemplary process, generally referred to using the numeral 1900, for providing a designated user perception adjustment using, at least in part, a dynamic LFSL. In this example, the process 1900 comprises input of parameters 1902 required by a light field display system, such as the type of LFSL employed (e.g. MLA, parallax barrier, or the like), and any required parameters related thereto or related to the display system. The scope and nature of parameters required as input for displaying perception-adjusted content will be understood by the skilled artisan. In some embodiments, where pupil location(s) or view zones are not input or received with parameters 1902, the pupil location may be determined, for instance using eye tracking processes, in process step 1904. Further, any designated perception adjustments 1904 (e.g. dioptric correction), or range thereof 1904, may further be received the display system or a processor associated therewith.

In accordance with various embodiments, the process 1900 may comprise any one or more ray tracing processes 1906 to compute display content according to any or all of the input parameters 1902, 1904, and 1906. Further, any ray tracing processes may further consider a LFSL position, as described above, which may, in accordance with various embodiments, comprise a range of potential positions that may be calculated 1908 to be optimal or preferred in view of, for instance, the range of dioptric corrections 1904 required. Accordingly, ray tracing 1906 and LFSL position 1908 calculations may be solved according to the Equations described above, or iteratively calculated based on, for instance, system constraints and/or input parameters. Upon determining a preferred LFSL position (e.g. distance from the display screen), the system may then position 1910 the LFSL, for instance via one or more automated actuators, at the preferred position for display 1912 of the perception adjusted content.

The skilled artisan will appreciate that the process steps depicted in the exemplary embodiment of FIG. 19 may, in accordance with various embodiments, be performed in various orders without departing from the scope or nature of the disclosure. Further, any or all process steps may be performed at the initiation of a viewing session, or may be periodically or continuously updated throughout viewing. For instance, viewing parameters 1904 may be updated as eye tracking processes determine a new viewing location, which may initiate calculation 1908 of and displacement 1910 to a new corresponding LFSL position. Similarly, should a different dioptric adjustment be preferred, for instance during the course of an eye examination, a LFSL may be displaced 1910 during display of content 1912. Further, and in accordance with various embodiments, adjustment 1910 of the LFSL may be performed in an iterative or feedback-based process, whereby the LFSL may be adjusted, for instance in real-time, to improve or optimise a display-to-LFSL distance during operation.

Generally, a LFSL position may be selected (e.g. calculated 1908) or adjusted based on the application at hand. Similarly, system specifications (e.g. the resolution of a pixelated display screen, the pitch and/or focal length of lenslets of a microlens array, or the like) may be selected based on the particular needs of an assessment or display system. For example, and in accordance with one embodiment, a given eye examination may require a relatively large range of dioptric power adjustments (e.g. 20+ diopter range), while also requiring a visual content quality parameter (e.g. visual content provided with resolution of 1 arcminute angular pitch or smaller, a view zone width, or the like). A system operable to this end may therefore comprise different components or configurations than a system that, for instance, is designed to be portable and is subject to various weight and/or size restrictions. For example, and in accordance with one embodiment, a portable system for performing saccade and/or vergence tests to assess for a cognitive impairment may require lower resolution and/or range of accessible perception adjustments than is required for a stationary eye examination system. Further, a portable system may be further limited in the amount of space over which a LFSL may be translated to affect a depth-based perception adjustment. Accordingly, various embodiments relate to systems and methods for providing perception adjustments with components and/or ranges of LFSL translation that are selected for a particular application.

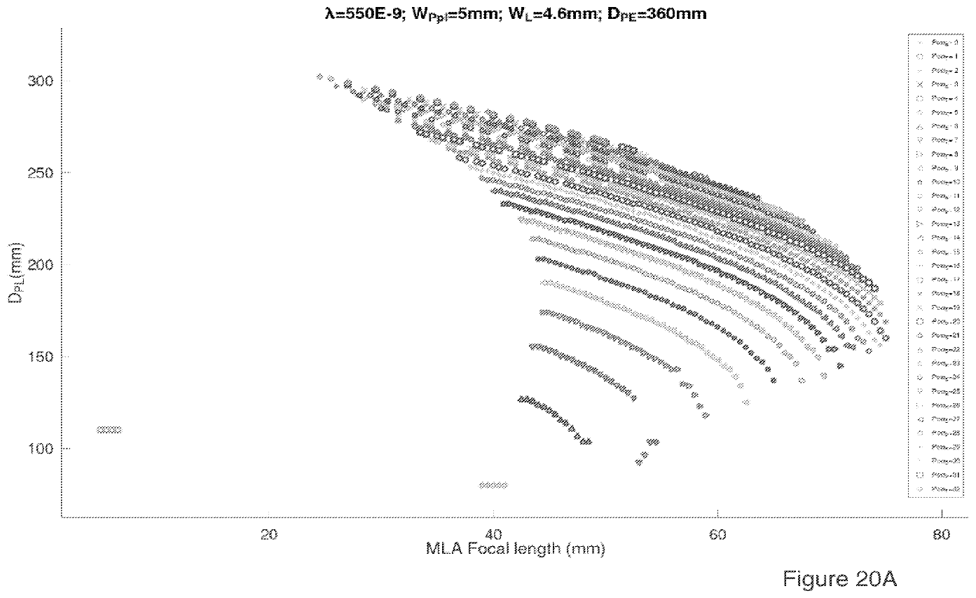

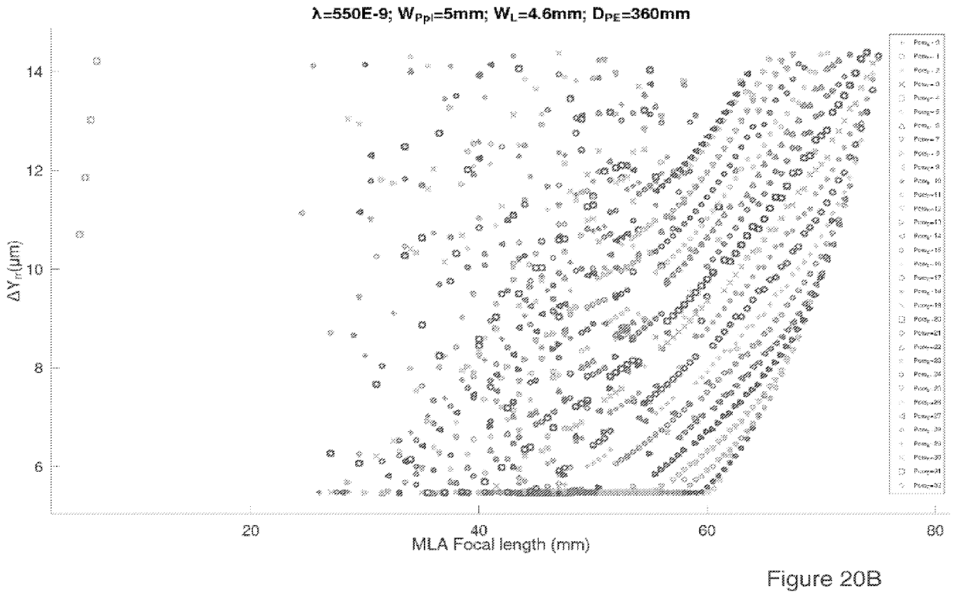

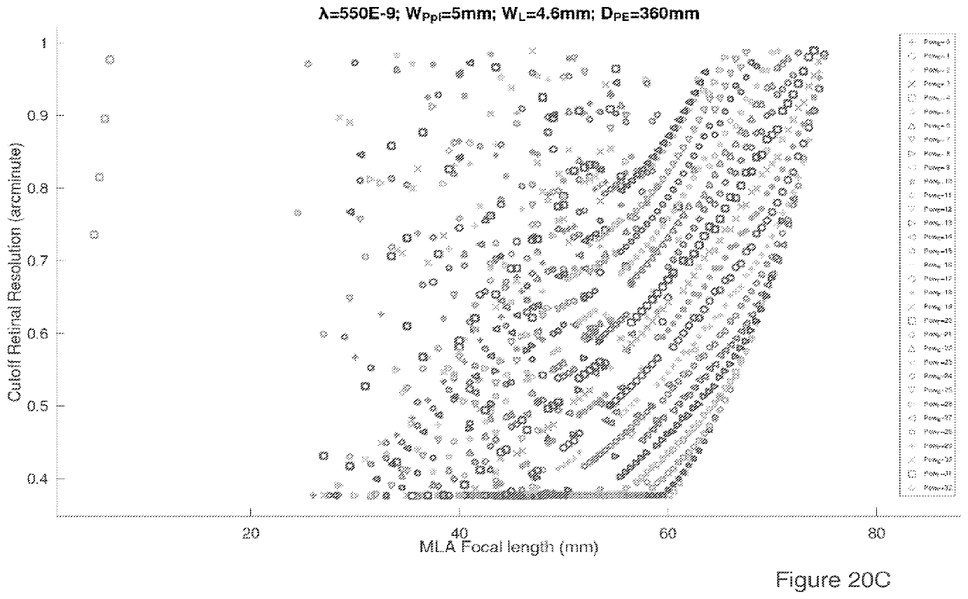

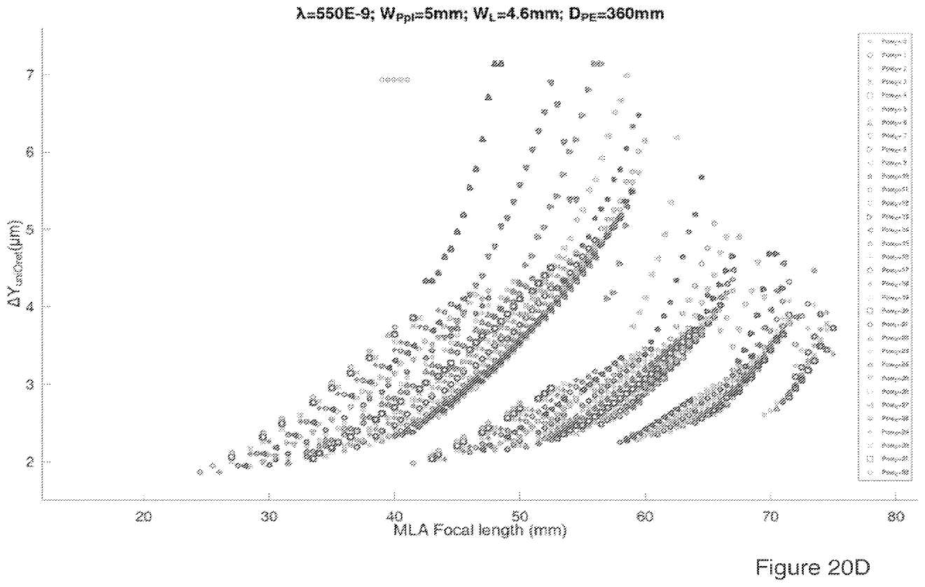

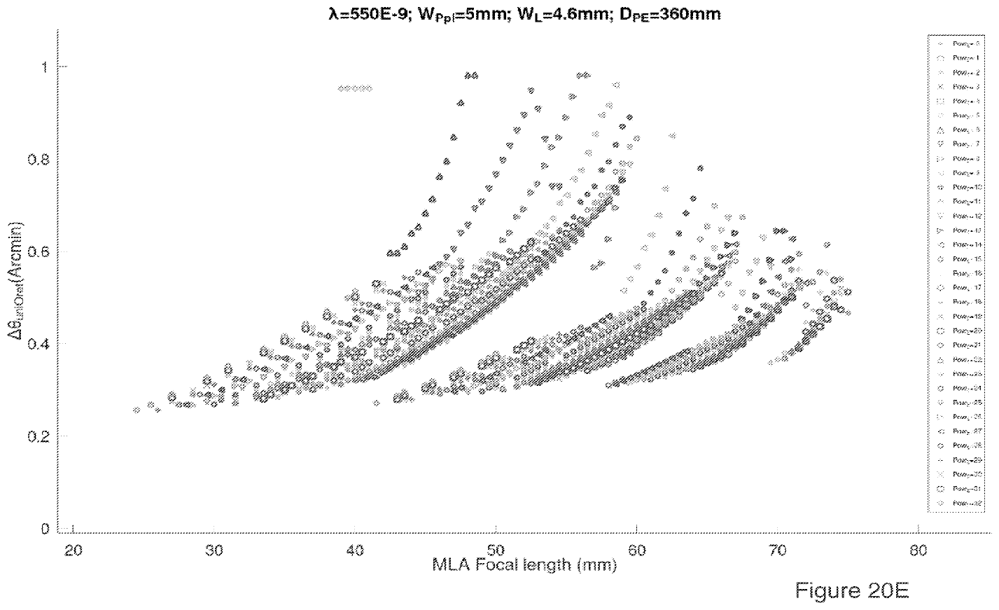

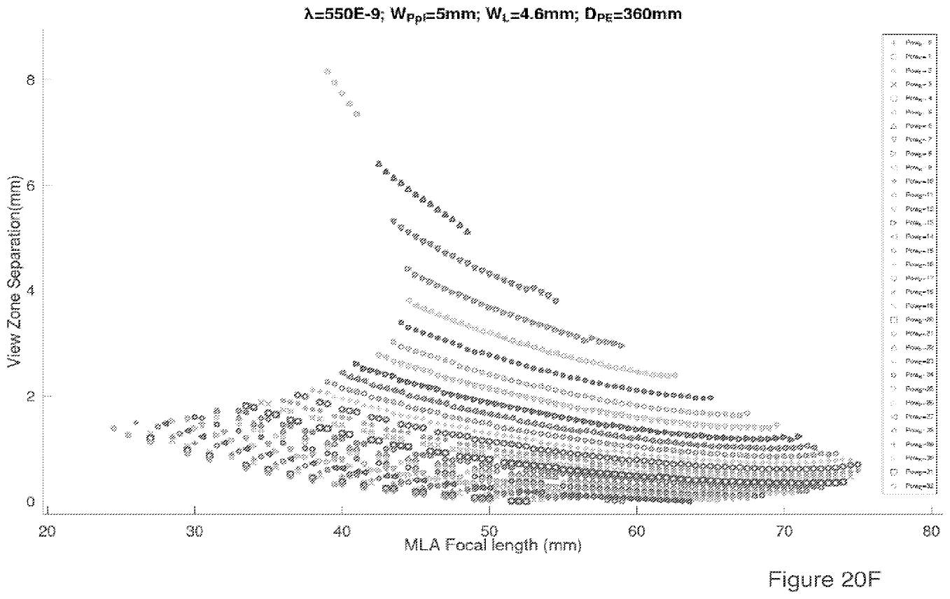

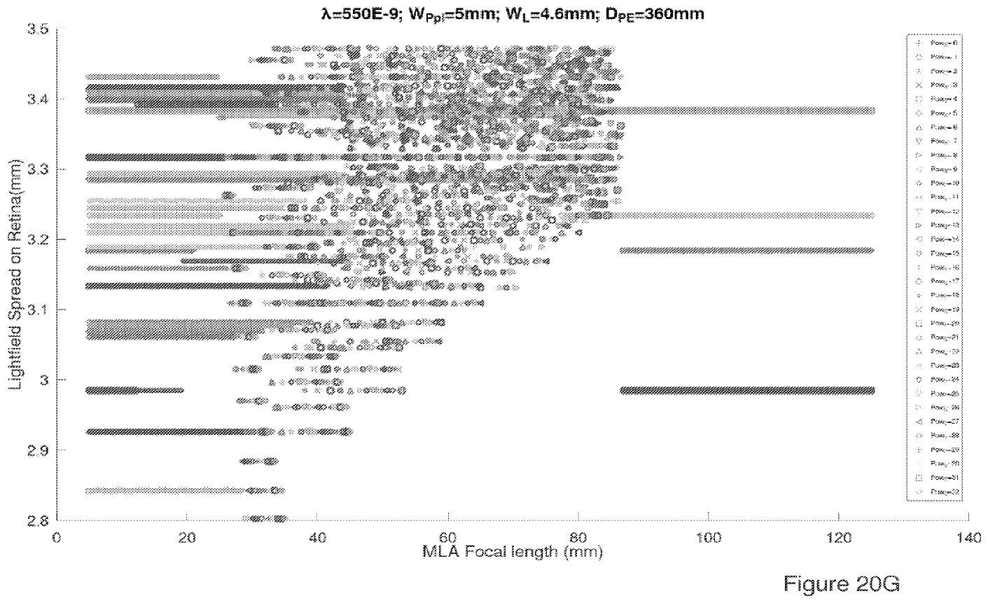

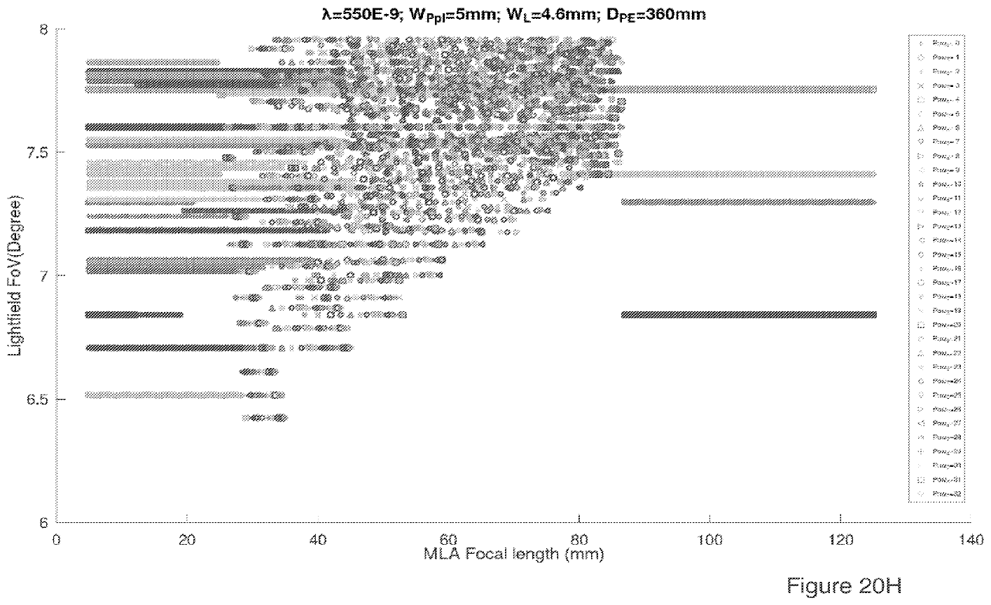

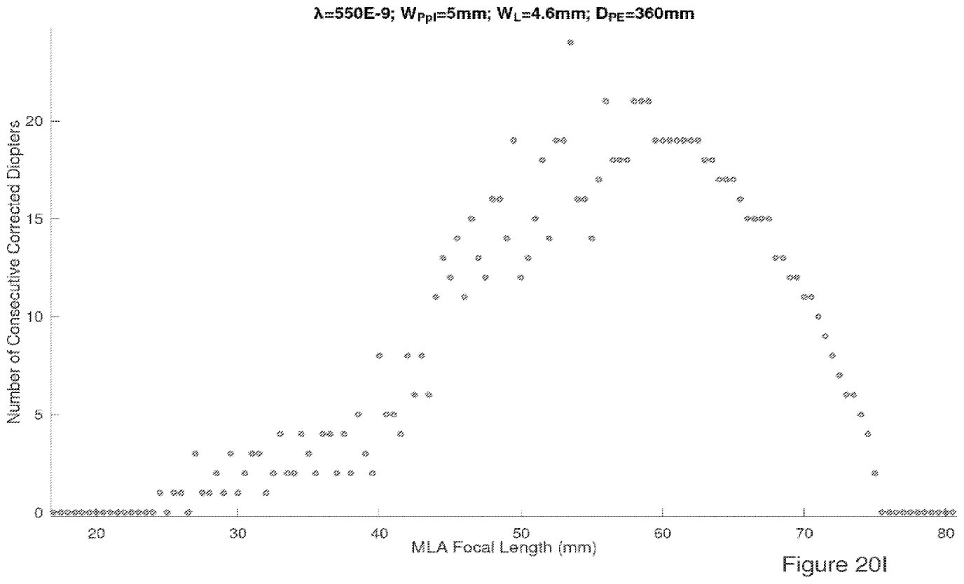

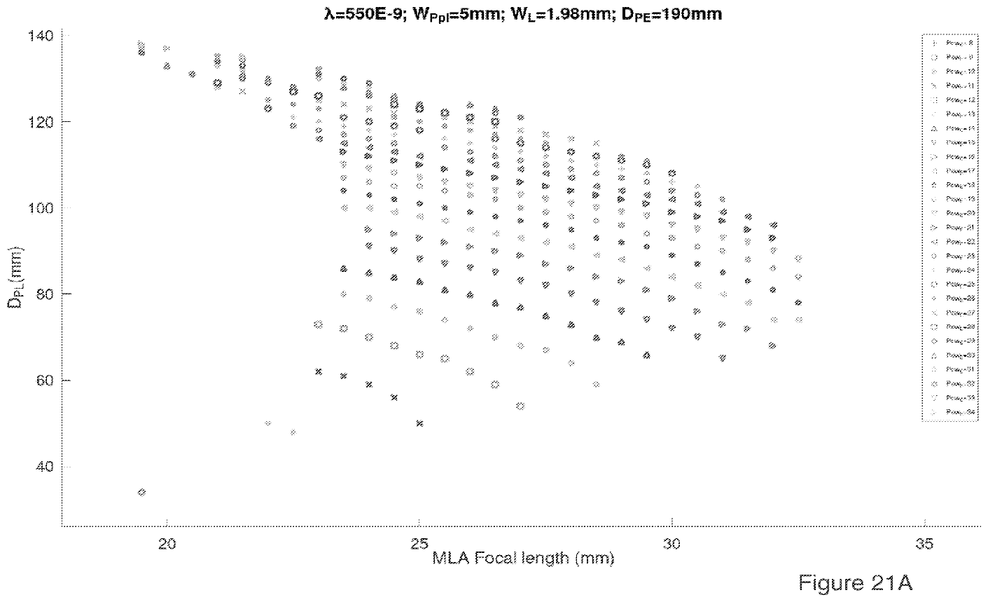

To this end, FIGS. 20A to 23J are exemplary plots illustrating non-limiting parameter spaces for exemplary light field shaping system configurations. It will be appreciated that, in accordance with various embodiments, system configurations and/or component specifications may enable, for instance, different perception adjustments or ranges thereof in view of one or more system or application parameters. For example, an MLA having a given lenslet pitch and focal length may enable a distinct range of perception adjustments for visual content (e.g. shifts in dioptric powers) at or below a required resolution threshold for a distinct system geometry (e.g. a particular LFSL position). If a perception adjustment is then required during, for instance, an eye examination or cognitive impairment assessment that is outside of that distinct range in view of, for instance, the resolution requirement for the presented content, the LFSL position may then be adjusted to select a new distinct geometry corresponding to a new distinct range of perception adjustments.

It will be appreciated that such distinct ranges of perception adjustments may or may not overlap, in accordance with different embodiments. For example, a first distinct range of perception adjustments corresponding to a first geometry may enable a dioptric power range of −5 to ±5 diopters, while a second distinct range of perception adjustments corresponding to a second geometry may enable a dioptric power range of −1 to ±8 diopters. Accordingly, a system geometry may be selected to, for instance, select the entire range of perception adjustments required for a particular application (e.g. a vision-based cognitive assessment). In accordance with another embodiment, a plurality of system geometries may be selected over the course of an examination in order to select different perception adjustment ranges which, together, enable the entire range of perception adjustments required for an assessment.

In accordance with one exemplary embodiment, FIGS. 20A to 201 illustrate various exemplary parameters simulated as a function of MLA focal length for various dioptric powers accessible to a light field system having a particular configuration and component specifications. This exemplary embodiment relates to a LFSL and display that may be arranged to maximise the range of dioptric corrections that may be obtained by a translating LFSL (in this case an MLA), as described above. In this example, the display screen comprises a Sharp™ display screen (51.84 mm×51.84 mm active area) with a 24 μm pixel pitch. As illustrated in FIG. 20I, the maximal range of continuous dioptric corrections that is achievable with this system while maintaining a cut-off resolution of 1 arcminute angular pitch or less is 23 diopters. This maximal range was obtained with an MLA characterised by an ˜53.5 mm focal length and a 4.6 mm pitch, while a 360 mm eye-to-display distance was maintained. While this 23-diopter range corresponds to a dioptric correction of an eye power error between 7 to 30 diopters, this range may be shifted to, for instance, correct an eye error within a different dioptric range using an additional optical component. For example, and in accordance with one embodiment, a 15-diopter lens placed in front of the eye of a user may enable the presentation of visual content with a perception adjustment corresponding to an eye dioptric error of −8 to 15 diopters, thereby enabling, for instance, an eye examination in this range. In accordance with another embodiment, a tunable lens may be employed to selectively increase or decrease a perception adjustment based on the needs of a particular assessment.

In this example, FIG. 20A is a plot of the display-to-MLA distance as a function of MLA focal length for different dioptric powers, highlighting the ability of a translatable LFSL for enabling a selection of a preferred perception adjustment by selecting or adjusting a system geometry. FIG. 20B is a plot of the corresponding focus spot size at the retina. FIG. 20C is a plot of the corresponding cut-off angular pitch/resolution values as a function of MLA focal length. FIG. 20D is a plot of the uniform spatial pitch between focused beams on the retina, while FIG. 20E is a plot of the corresponding cut-off resolution. FIG. 20F is a plot of view zone separation from a reference pupil of 5 mm diameter, while FIG. 20G is a plot of spread on the retina. FIG. 20H is a plot of the corresponding field of view as a function of MLA focal length. Finally, FIG. 20I is a plot of the maximum number of continuous dioptric powers (with 1 diopter step) that can be corrected for by changing the MLA-to-display distance.

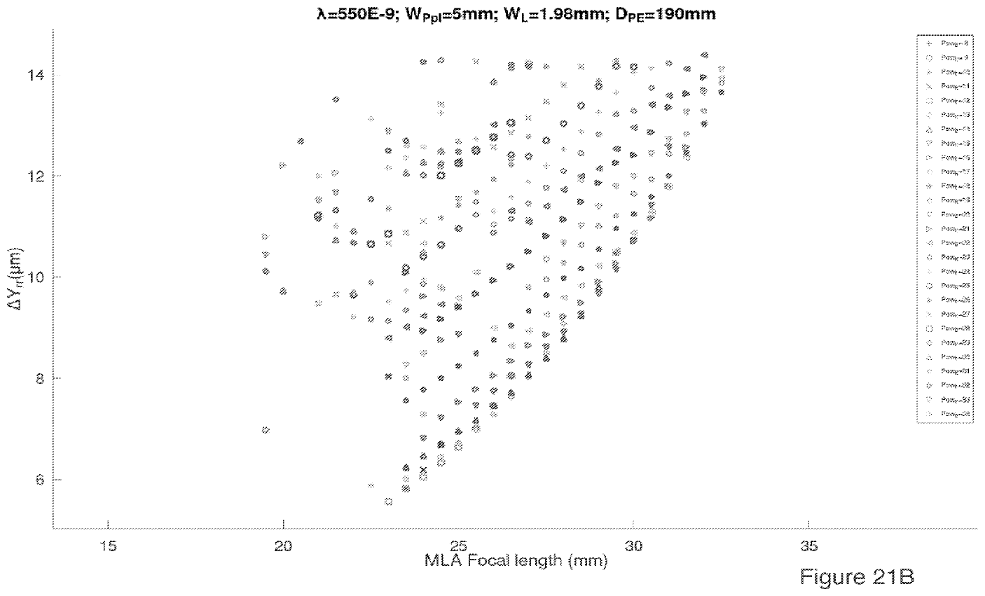

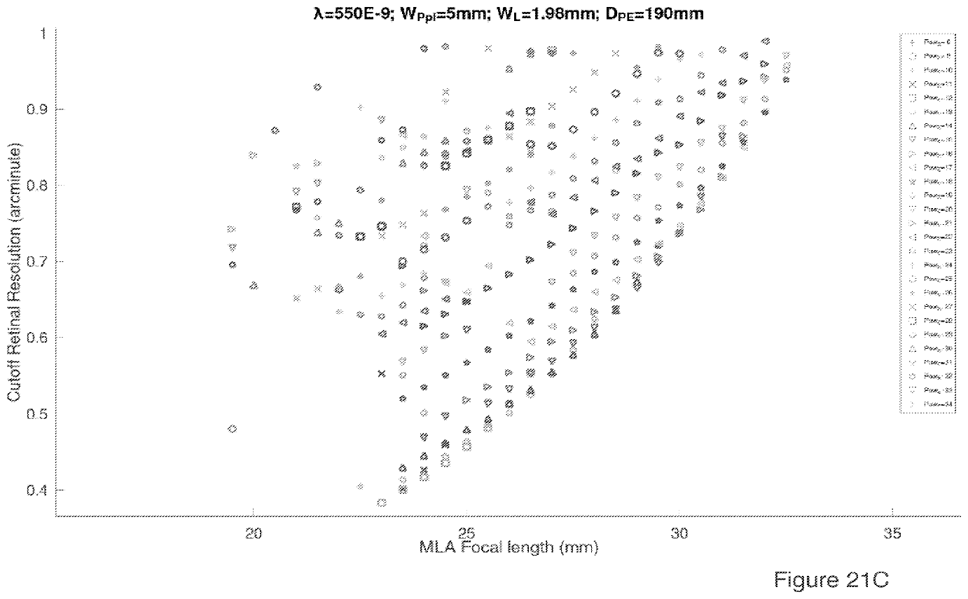

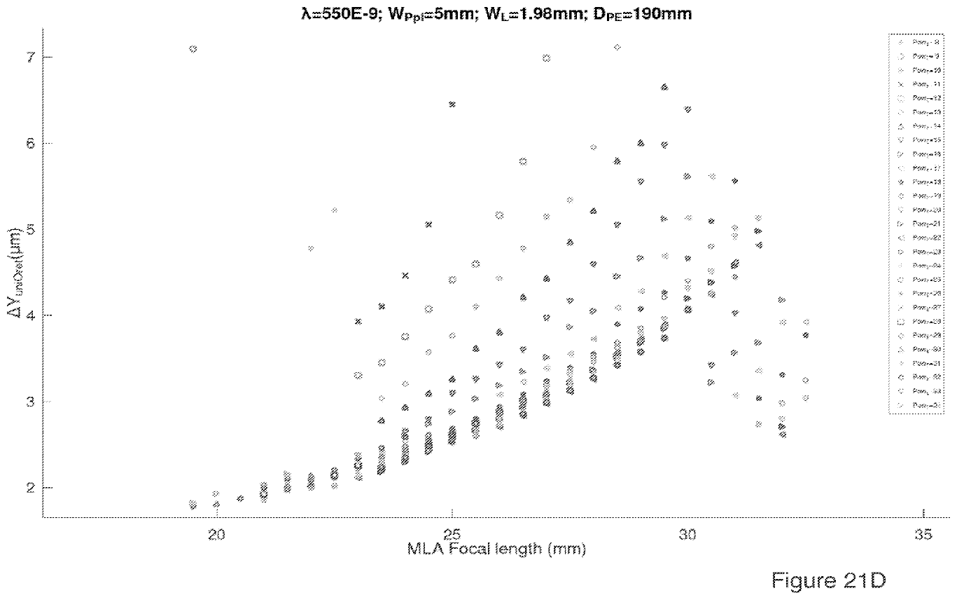

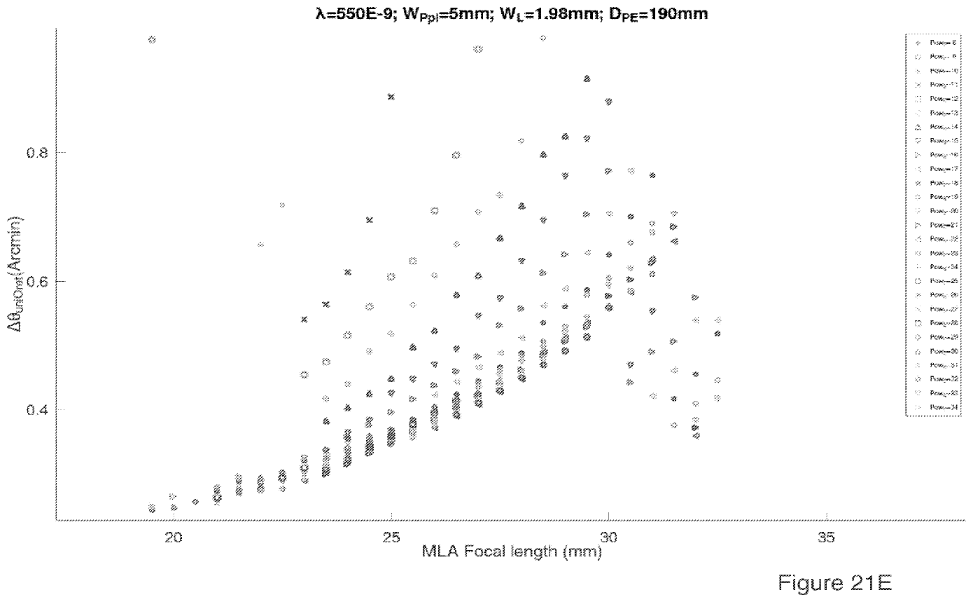

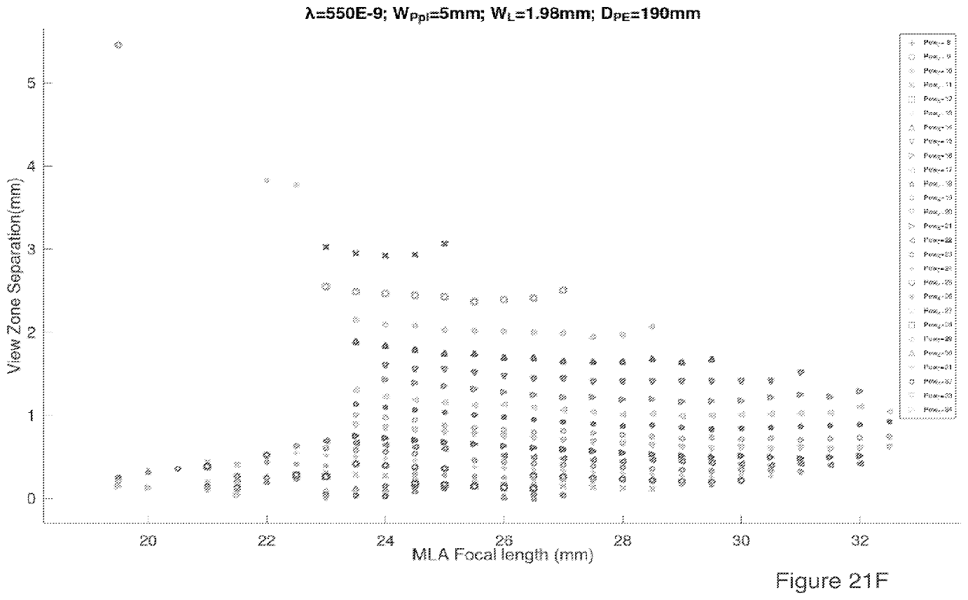

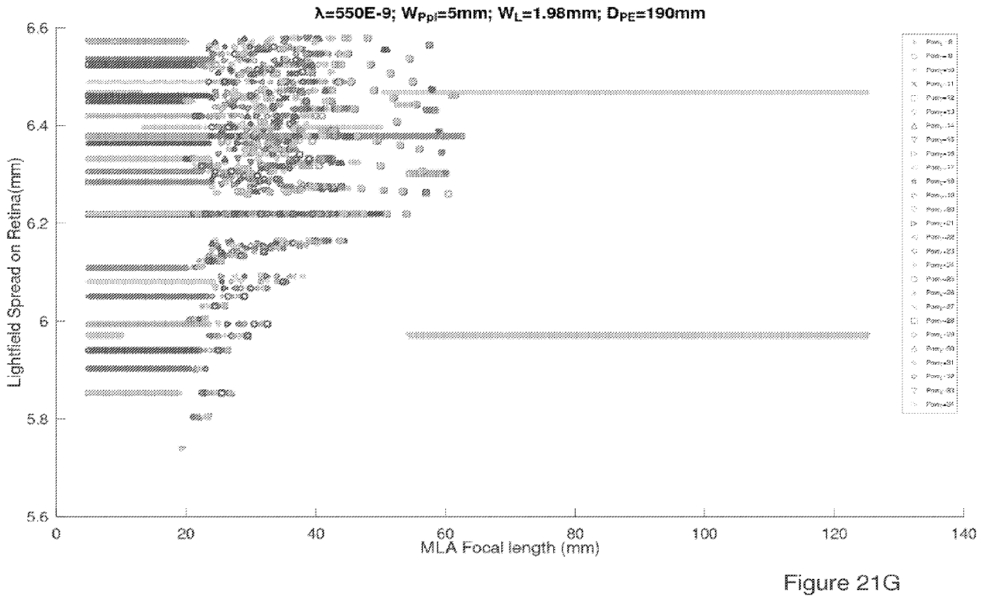

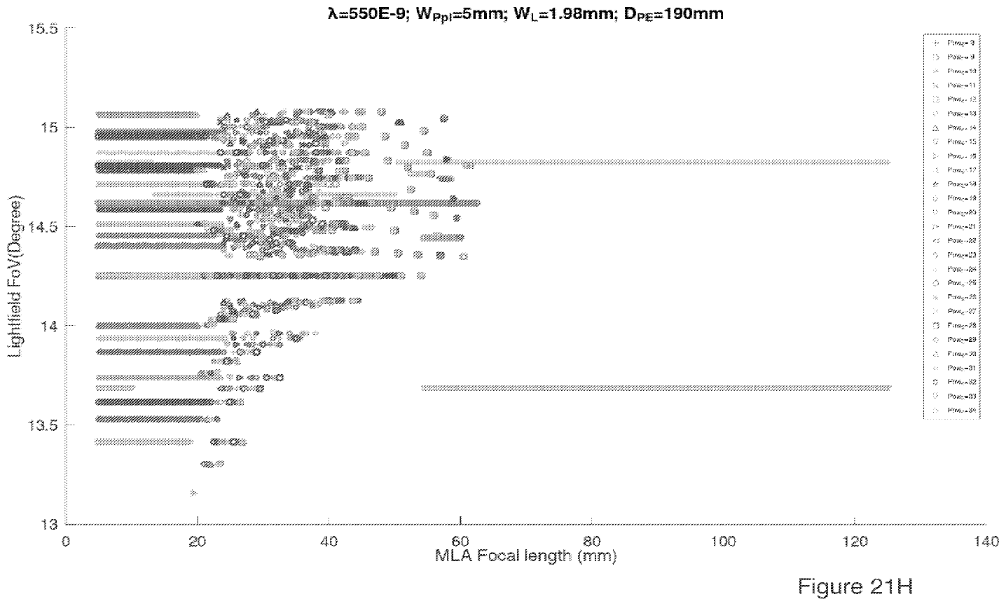

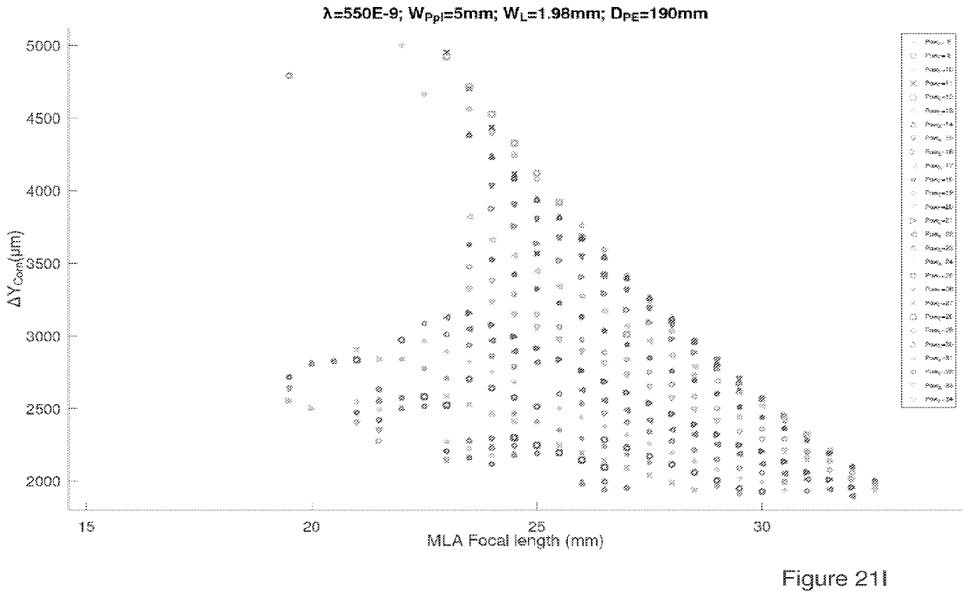

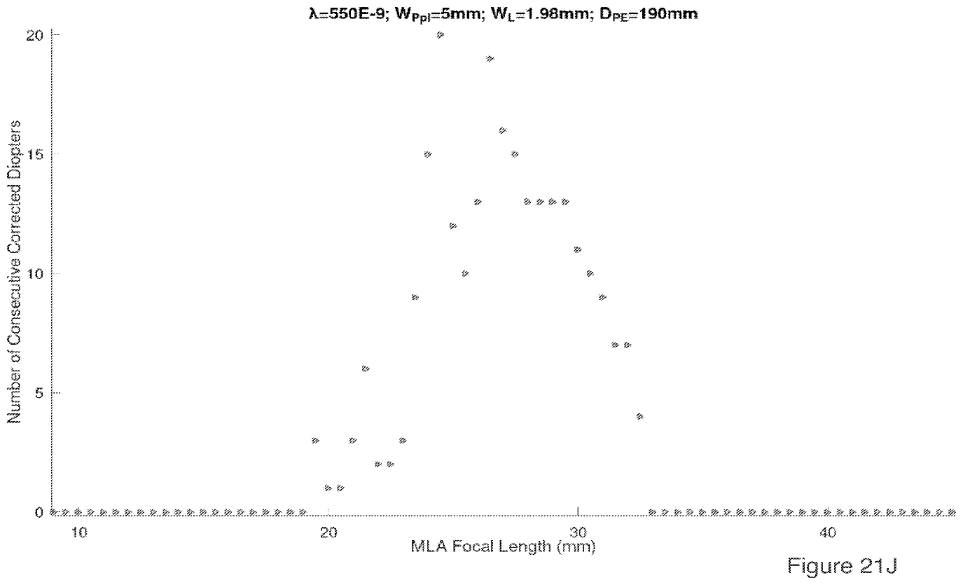

In accordance with another embodiment, FIGS. 21A to 21J illustrate various exemplary parameters simulated as a function of MLA focal length for various dioptric powers accessible to a different light field system having a different configuration and different component specifications. In this example, the LFSL layer comprises an MLA having a pitch of 1.98 mm and focal length of ˜24.5 mm, while the eye-to-display distance was selected as 190 mm. The system again comprised a display with 24 μm pixel pitch. In this example, a maximum continuous span of diopters that was achievable at 1 arcminute or less angular pitch/resolution was 19 diopters, corresponding to perception adjustments between 11 and 30 diopters. As described above, depending on the application at hand, an additional optical component may be introduced to the system to shift this range of dioptric corrections. For example, a 15-diopter lens placed in front of the eye of a user may enable correction for a range of eye power errors between-4 and 15 diopters.

In this example, FIG. 21A is a plot of the MLA-to-display distance as a function of MLA focal length for different eye power errors. FIG. 21B is a plot of the corresponding focus spot size at the retina. Similarly, FIG. 21C is a plot of the corresponding cut-off angular pitch/resolution values as a function of MLA focal length. FIG. 21D is a plot of the uniform spatial pitch between focused beams on the retina, while FIG. 21E is a plot of the corresponding angular pitch/resolution for a uniform distribution of focused beams on the retina. FIG. 21F is a plot of view zone separation from a reference 5 mm diameter pupil, while FIG. 21G is a plot of spread on the retina. FIG. 21H is a plot of the corresponding field of view as a function of MLA focal length. FIG. 21I is a plot of the beam size on the cornea. Finally, FIG. 21J is a plot of the maximum number of continuous dioptric power error points (spaced by 1 diopter steps) that can be corrected for by changing the MLA-to-display distance.

In addition to, or as an alternative to maintaining a threshold resolution of presented content, various other visual content quality parameters may be considered when selecting, for instance, light field system components (e.g. LFSL specifications) and/or geometries (e.g. LFSL position). For example, and without limitation, various embodiments relate to selecting system components and configurations in view of a corneal beam size constraint. For example, one embodiment relates to the selection of an MLA with specifications and a position within the system such that the beam size on the cornea of a user is maintained between 2.7 mm and 3.7 mm. This may, in accordance with one embodiment, enable a maximisation of focus range of retinal beam spots.

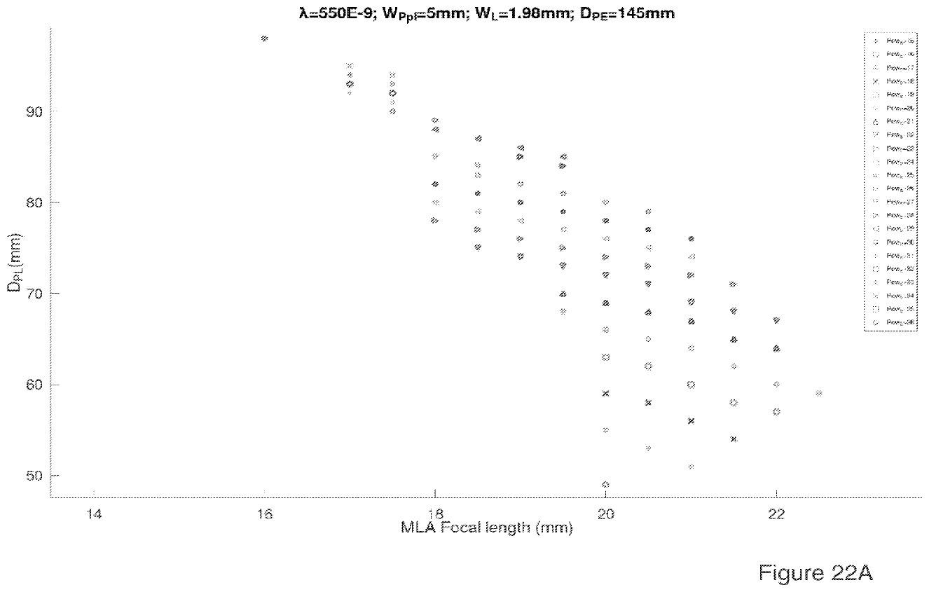

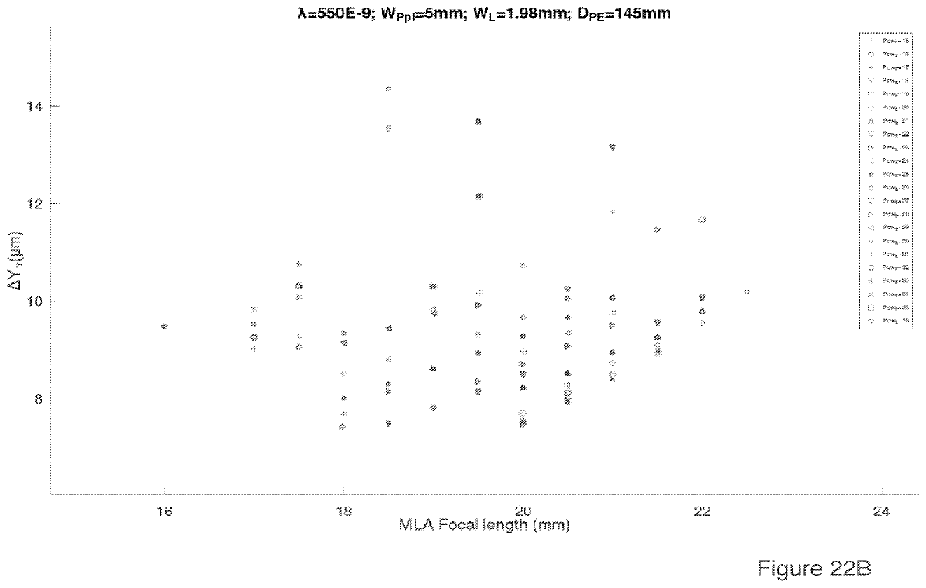

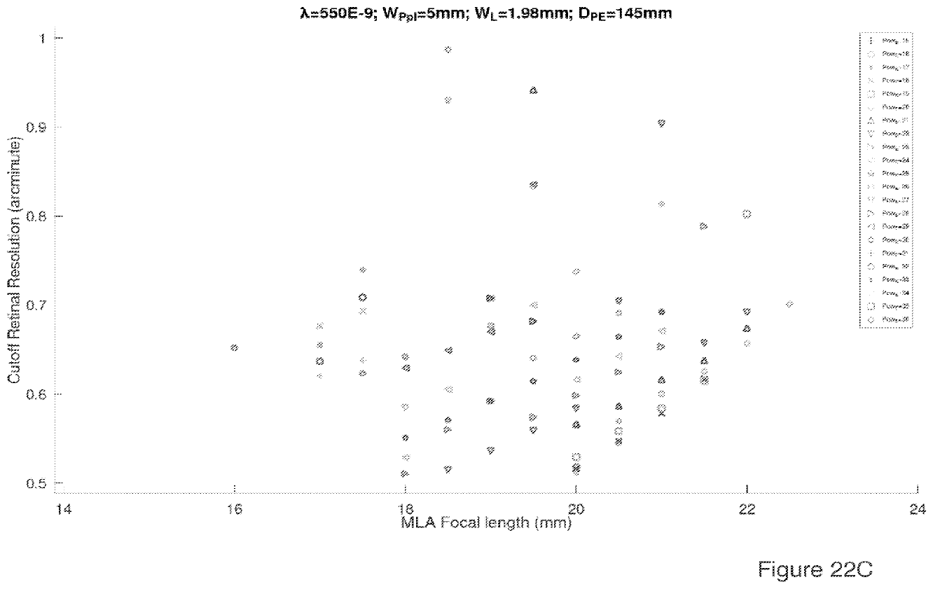

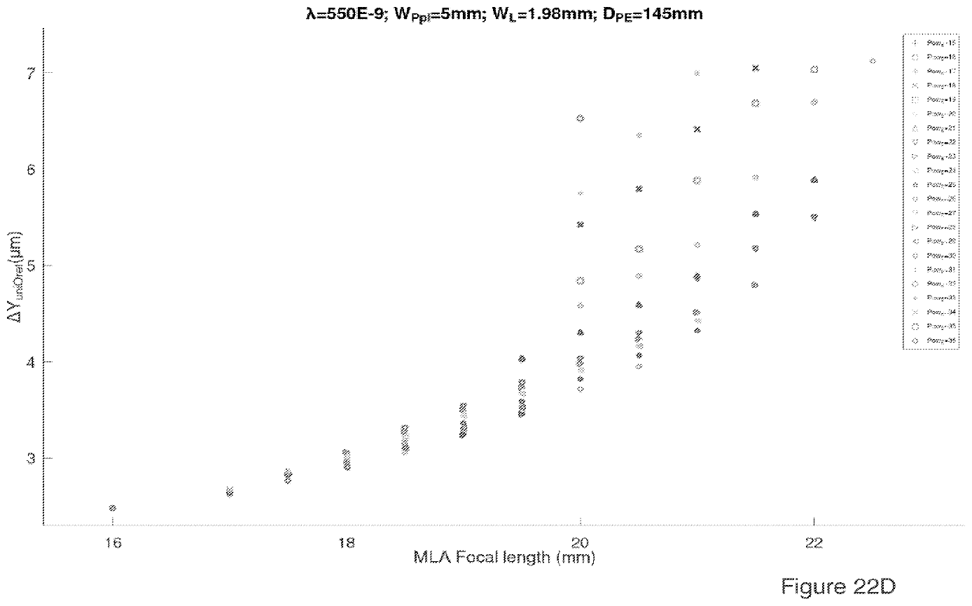

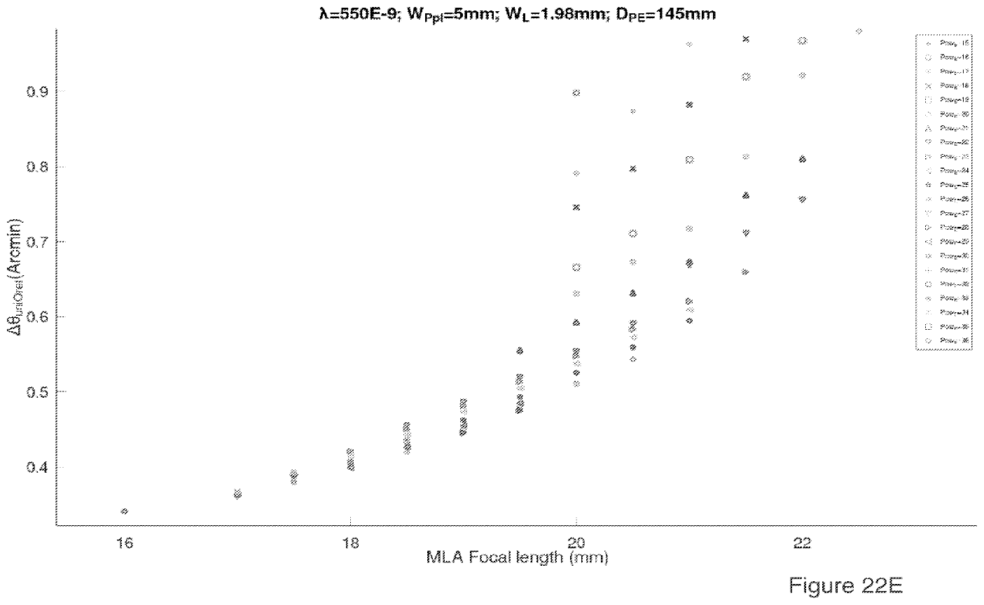

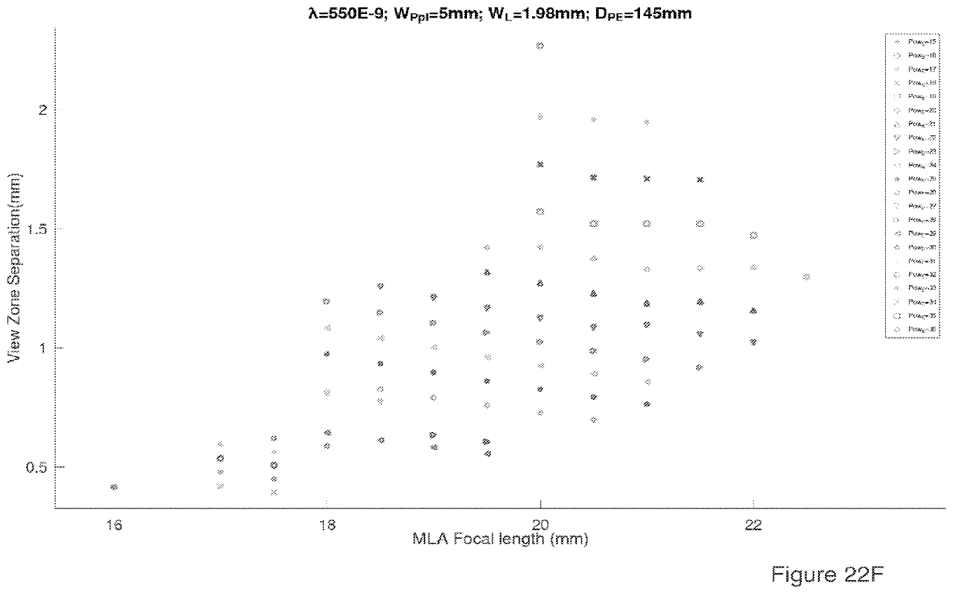

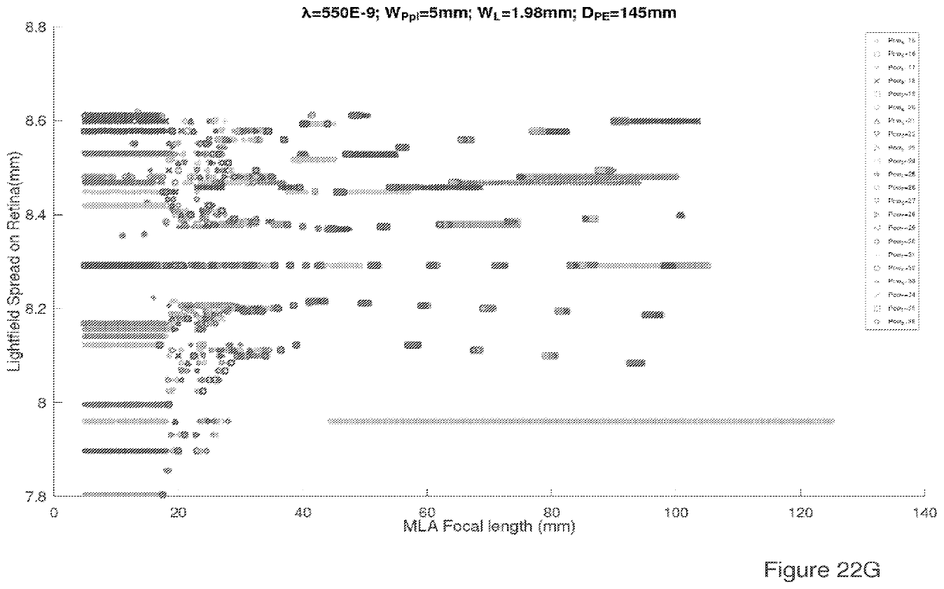

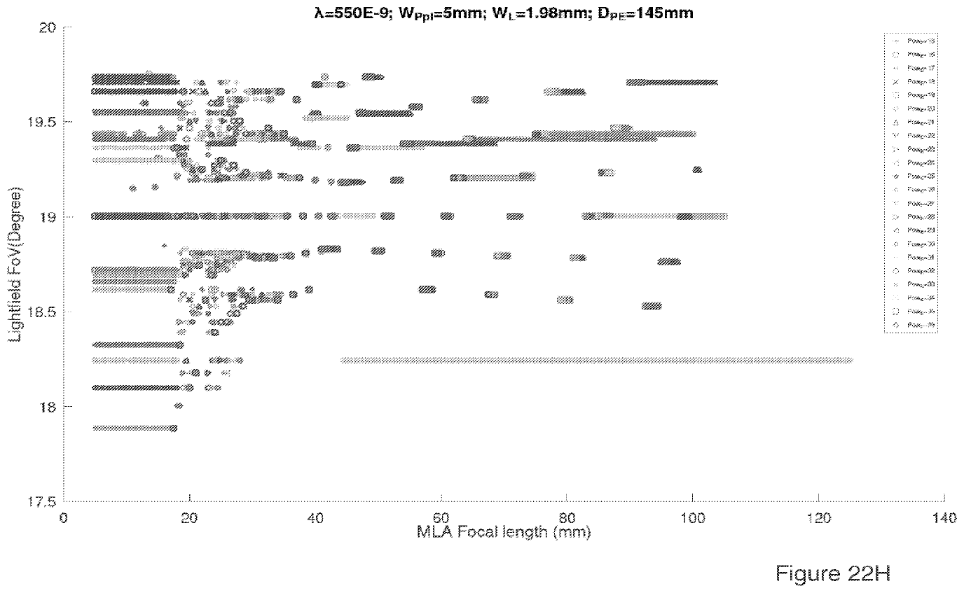

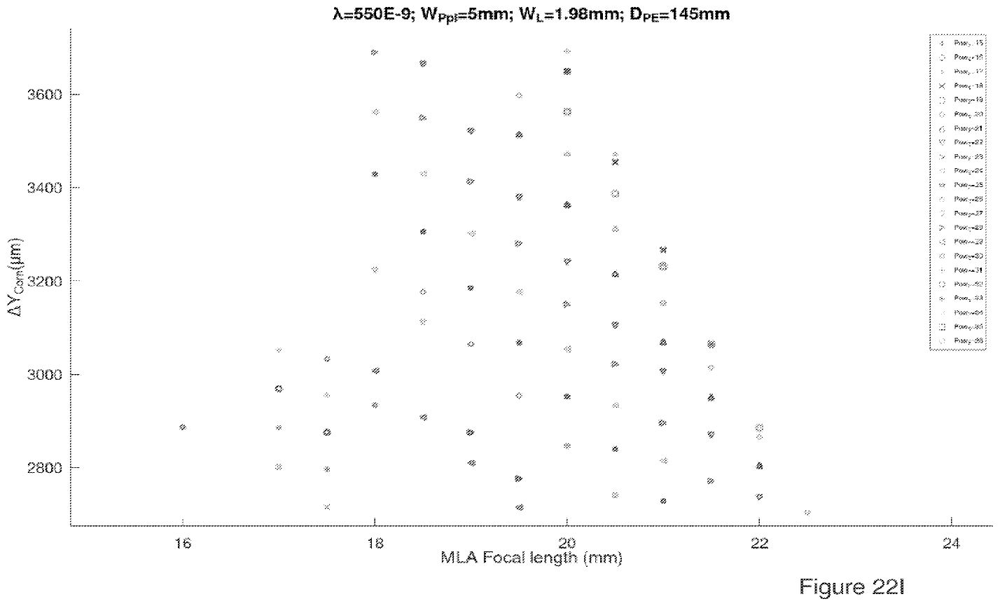

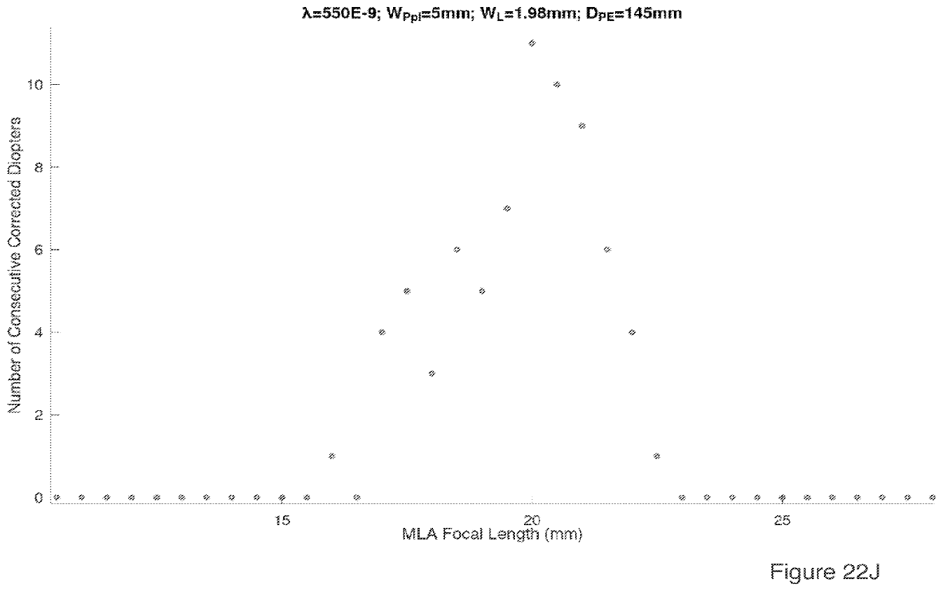

Applying this constraint, FIGS. 22A to 22J illustrate various exemplary parameters simulated as a function of MLA focal length for various dioptric powers accessible to an exemplary light field system. In this example, the display is characterised by a 24 μm pitch, the MLA selected comprised a lenslet pitch of 1.98 mm and focal length of ˜20 mm, and the system was characterised by an eye-to-display distance of 145 mm. In this example, a continuous range of 10 diopters was achieved, corresponding to a perception adjustment range of 16 to 26 diopters, which, again, may be shifted via the employ of an additional optical component.

In this exemplary embodiment. FIG. 22A is a plot of the MLA-to-display distance as a function of MLA focal length for different dioptric powers. FIG. 22B is a plot of the corresponding focus spot size at the retina. Similarly, FIG. 22C is a plot of the corresponding cut-off angular pitch/resolution values as a function of MLA focal length. FIG. 22D is a plot of the uniform spatial pitch between focused beams on the retina, while FIG. 22E is a plot of the corresponding angular pitch/resolution for a uniform distribution of focused beams on the retina. FIG. 22F is a plot of view zone separation from a reference 5 mm pupil, while FIG. 22G is a plot of spread on the retina. FIG. 22H is a plot of the corresponding field of view as a function of MLA focal length. FIG. 22I is a plot of the beam size on the cornea. Finally, FIG. 22J is a plot of the maximum number of continuous dioptric powers that can be corrected for by changing the MLA-to-display distance.

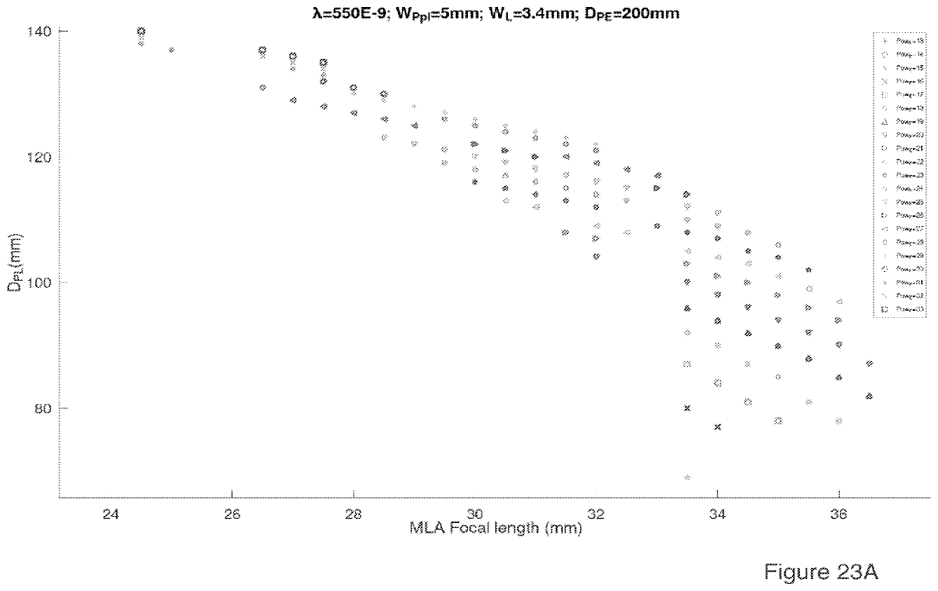

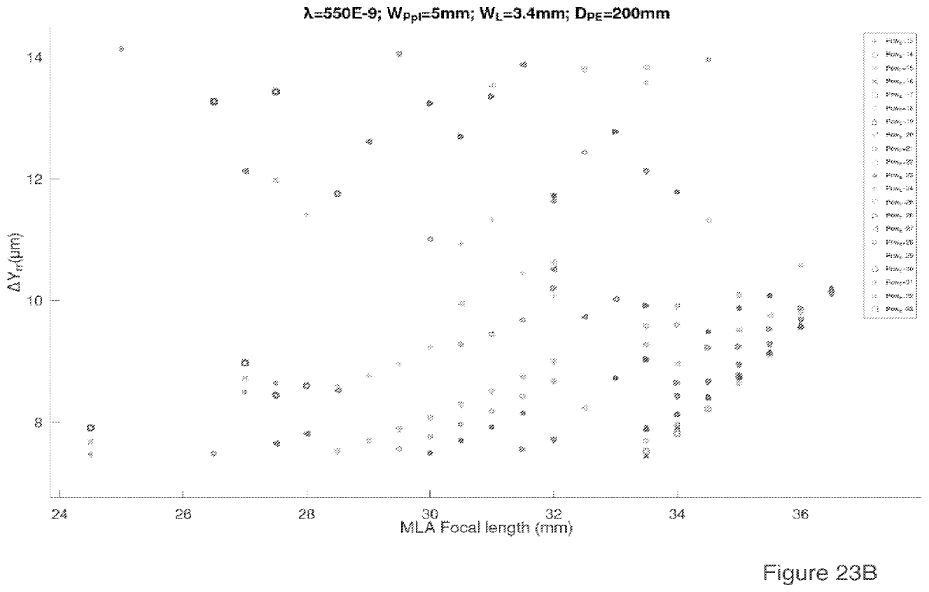

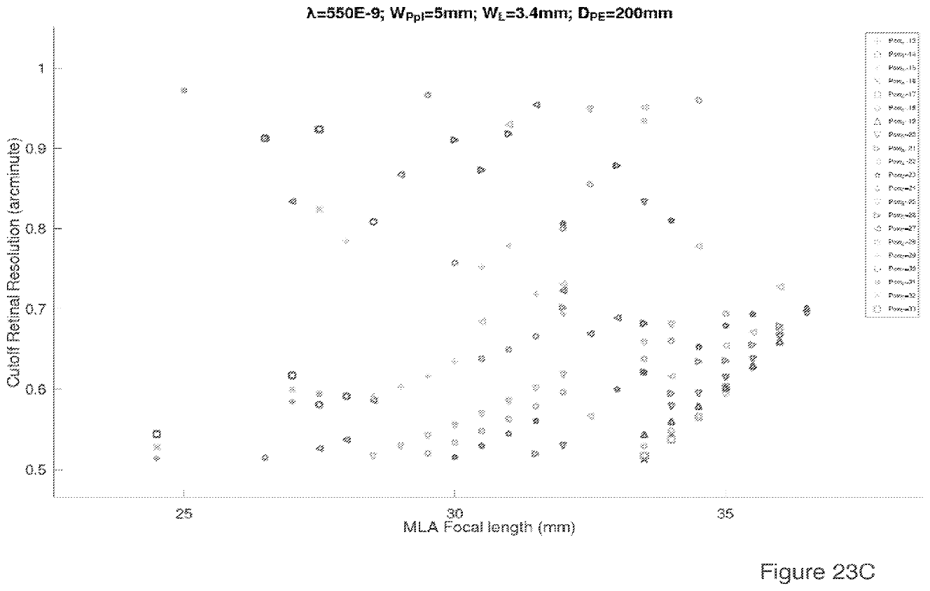

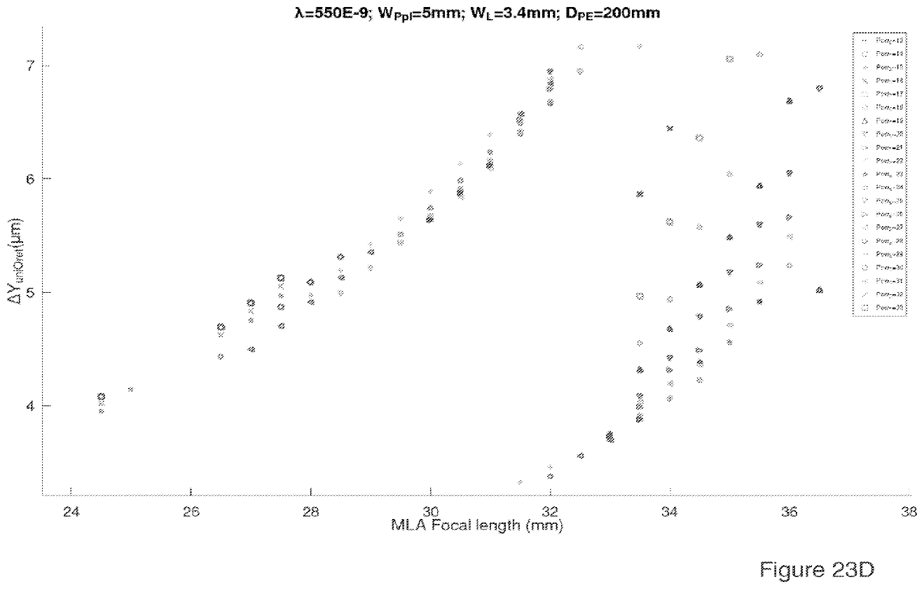

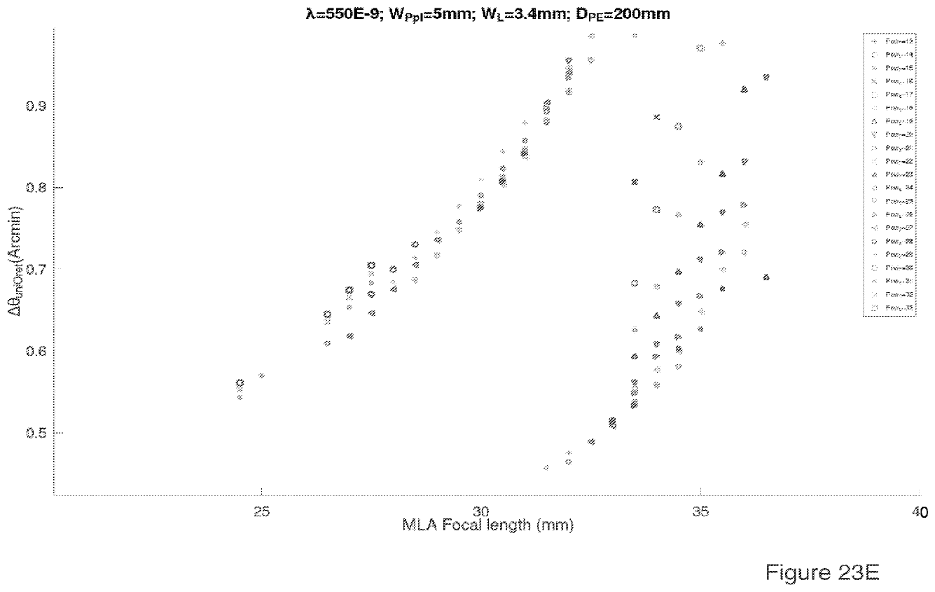

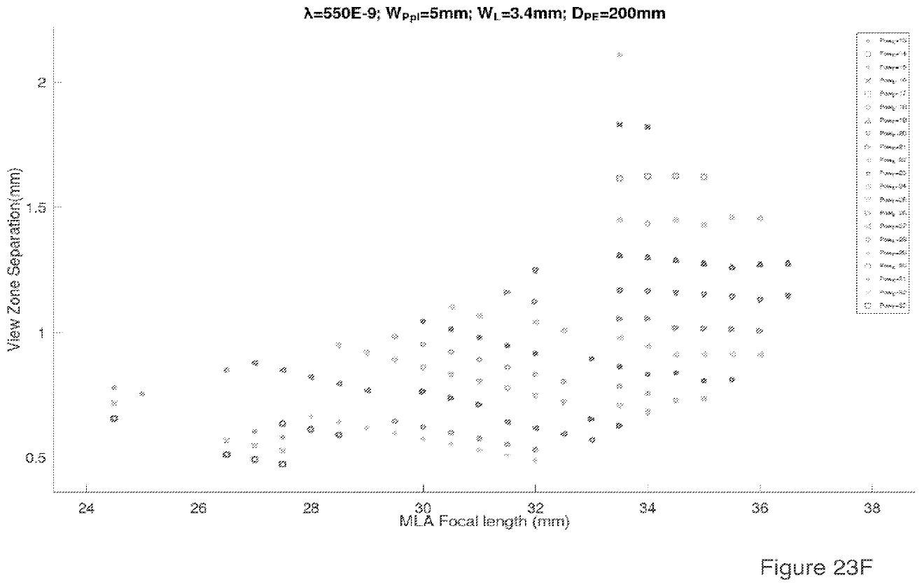

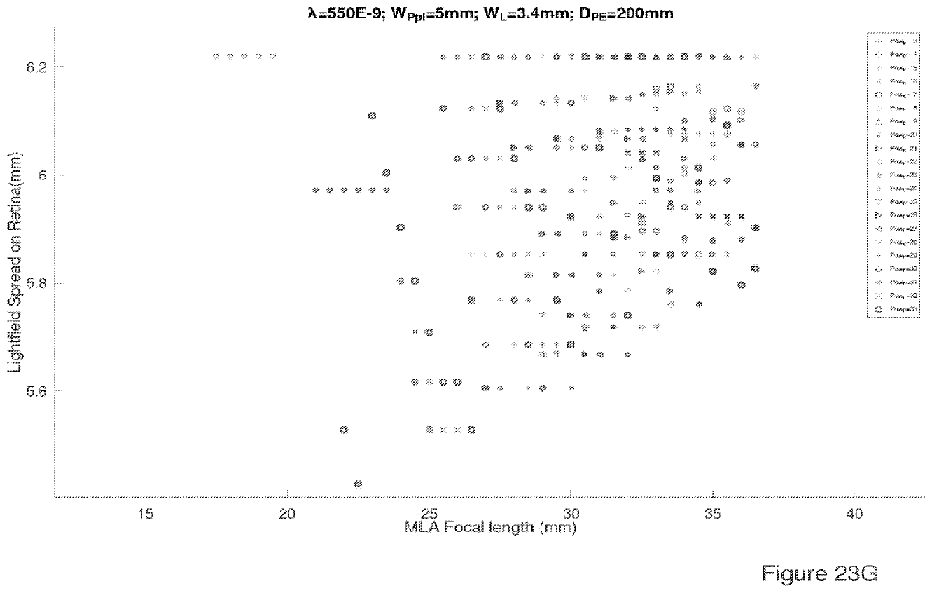

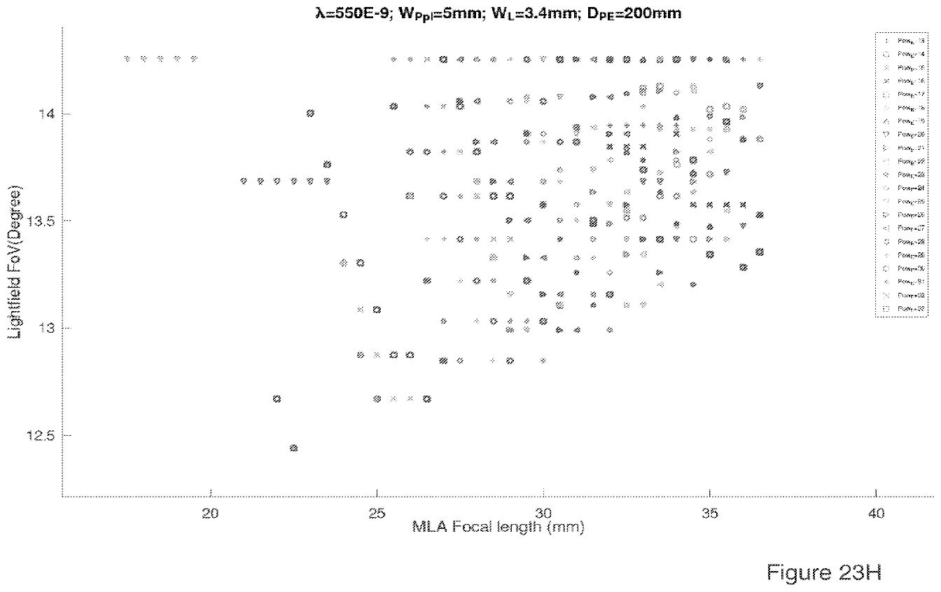

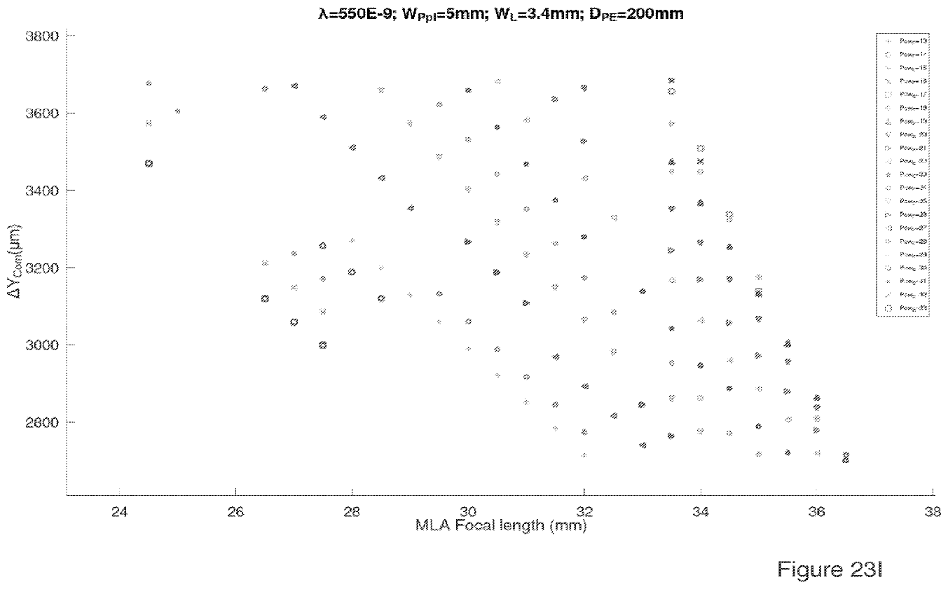

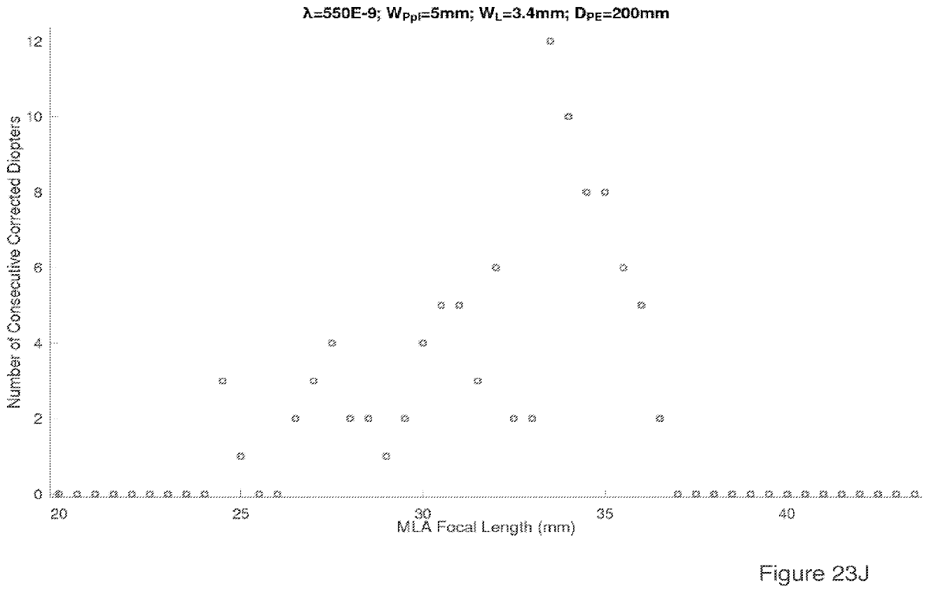

Again applying the visual content quality parameter constraints of maintaining a corneal beam size between 2.7 and 3.7 mm and a perception-adjusted content resolution of 1 arcminute angular pitch at most, FIGS. 23A to 23J illustrate various exemplary parameters simulated as a function of MLA focal length for various dioptric powers accessible to another exemplary light field system. In this example, the display again comprises a 24 μm pixel pitch, while the LFSL comprises an MLA characterised by a lenslet pitch of 3.4 mm and a focal length ˜34.5 mm. The eye-to-display distance is 200 mm. In this exemplary embodiment, a continuous range of 11 diopters is achievable, corresponding to perception adjustments between 15 and 26 diopters.

In this exemplary embodiment, FIG. 23A is a plot of the MLA-to-display distance as a function of MLA focal length for different dioptric powers. FIG. 23B is a plot of the corresponding focus spot size at the retina. Similarly, FIG. 23C is a plot of the corresponding cut-off angular pitch/resolution values as a function of MLA focal length. FIG. 23D is a plot of the uniform spatial pitch between focused beams on the retina, while FIG. 23E is a plot of the corresponding angular pitch/resolution for a uniform distribution of focused beams on the retina. FIG. 23F is a plot of view zone separation from 5 mm diameter reference pupil, while FIG. 23G is a plot of spread on the retina. FIG. 23H is a plot of the corresponding field of view as a function of MLA focal length. FIG. 23I is a plot of the beam size on the cornea. Finally, FIG. 23J is a plot of the maximum number of continuous dioptric power points (separated by 1 diopter) that can be corrected for by changing the MLA-to-display distance.

As described above, various system components and/or configurations may be employed depending on, for instance, the application at hand (e.g. a vision assessment, a cognitive impairment assessment, or the like), or a visual content quality parameter associated therewith. For example, a visual acuity examination may require a large range of dioptric corrections (e.g. −8 to ±8 diopters). Accordingly, a system designed to this end may comprise components and configurations similar to those described with respect to FIGS. 20A to 20I that relating to a continuous range of 23 diopters with at least 1 arcminute resolution. Conversely, a cognitive impairment assessment system operable to perform saccadic and vergence tests may have less strict content quality parameters associated therewith. For example, less resolution (larger angular pitch) may be required to generally follow a moving stimulus in a pursuit assessment, and a smaller range of dioptric corrections may be needed to perform a vergence test. Accordingly, a cognitive impairment assessment device may employ simpler and/or more cost-effective components than a visual acuity refractor/phoropter. Similarly, more compact or lightweight components may be employed in order to, for instance, make a cognitive impairment assessment system more portable for in-field assessments.

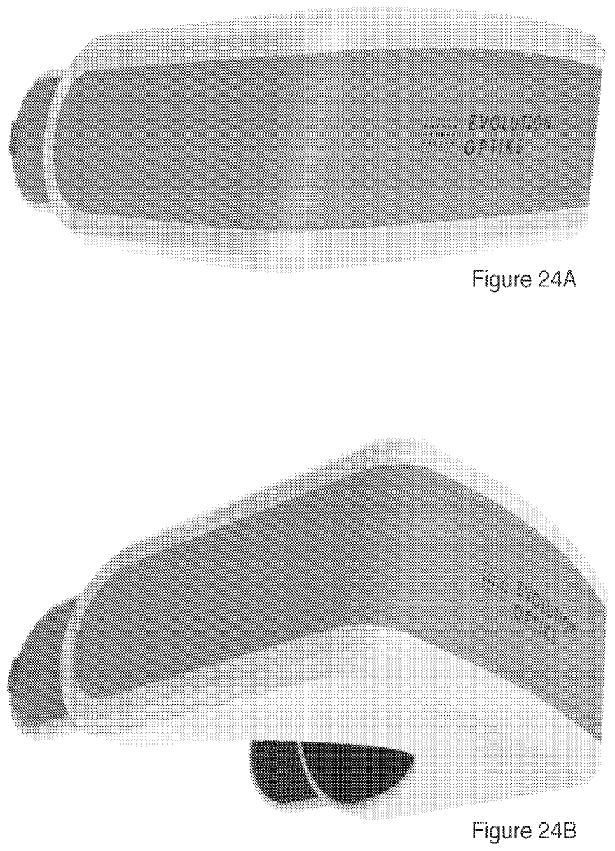

For example, FIGS. 24A and 24B show perspective views of a portable cognitive impairment assessment device. Due in part to its compact nature, the range of dioptric adjustments required for a vergence test may, in accordance with some embodiments, require that a LFSL be translated relative to a display screen.

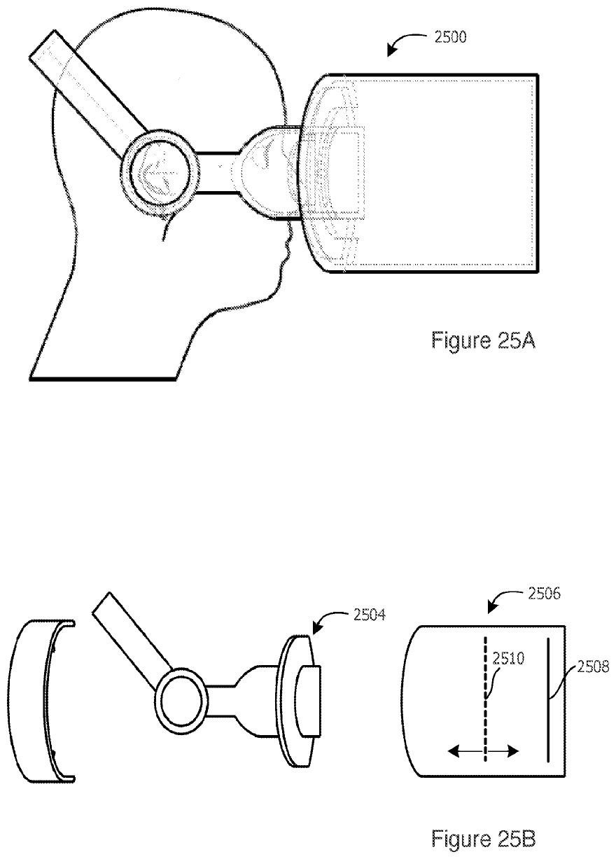

This is further schematically illustrated in FIGS. 25A and 25B. In this exemplary embodiment, a different cognitive impairment assessment device 2500 comprises a lightweight, user-interfacing potion 2504 and a load-bearing portion 2506. FIG. 25A shows the device 2500 while in use by a subject 2508, while FIG. 25B schematically shows an exploded view of various exemplary components of the device 2500. In this exemplary embodiment, the load-bearing portion 2506 comprises a pixelated display screen 2508 and a displaceable light field shaping layer (LFSL) 2510 disposed between the user and the display screen 2508 to shape a light field emanating therefrom, thereby enabling adjustable perception adjustments.

In accordance with various embodiments, various device configurations and components may be employed to perform a vision-based test.

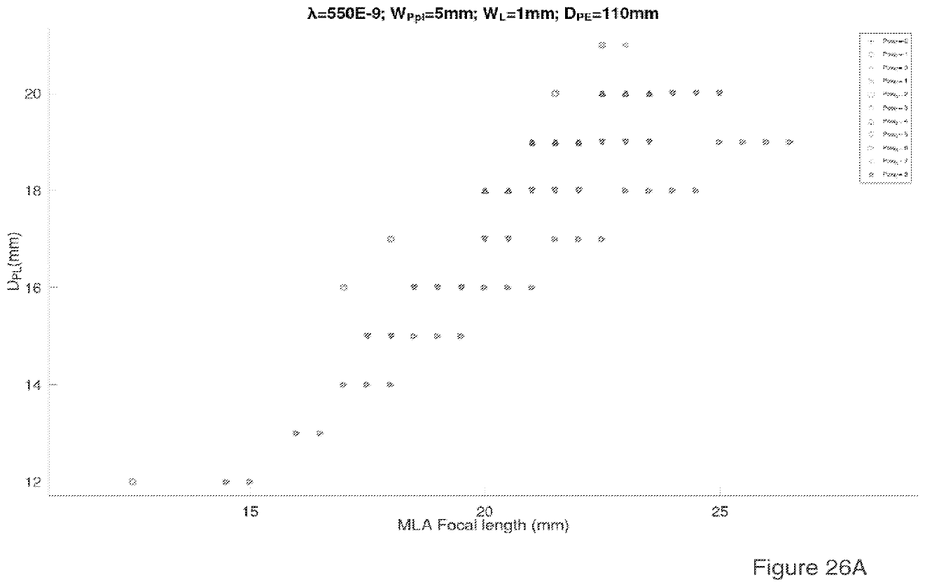

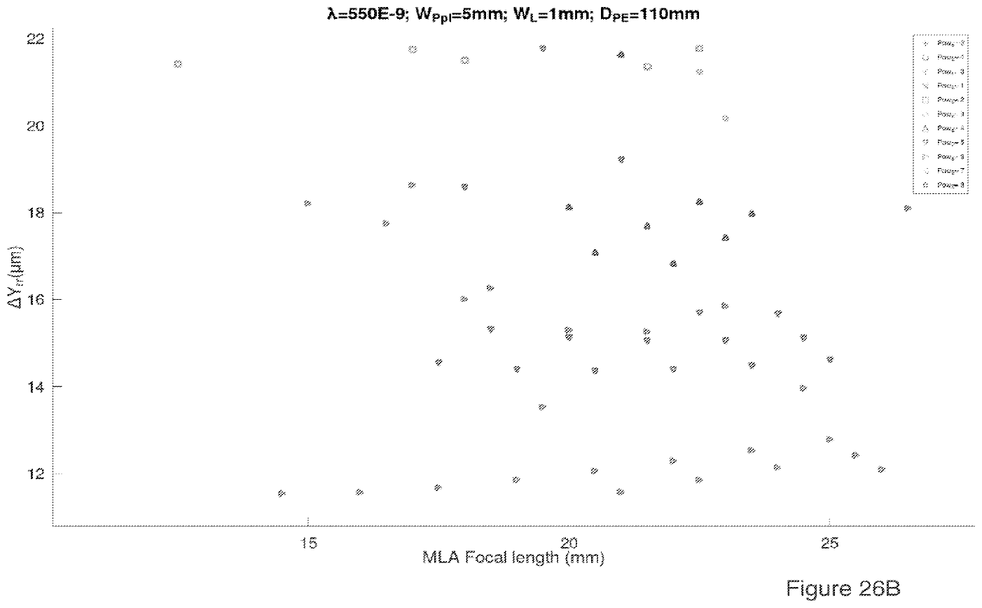

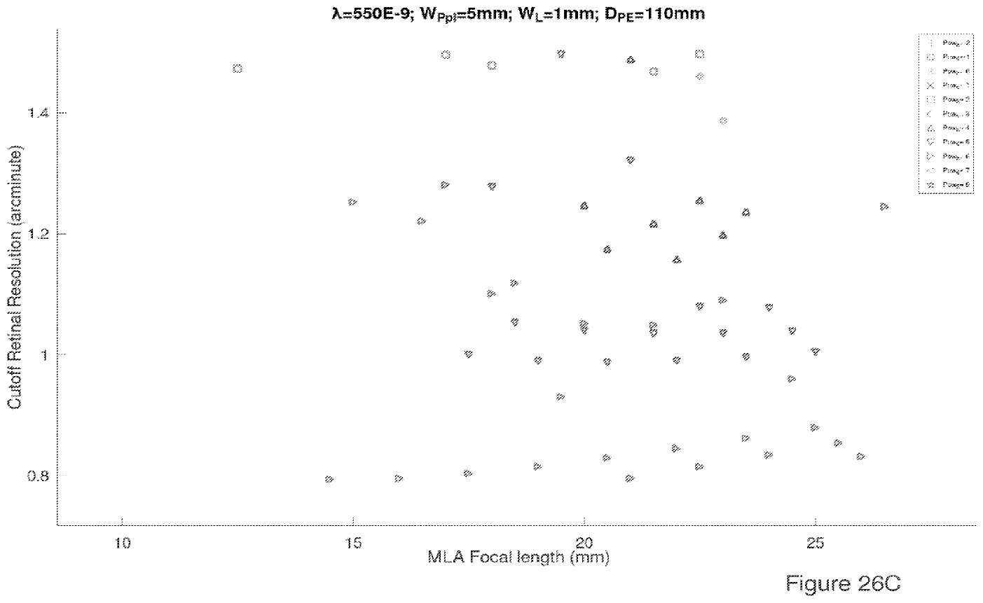

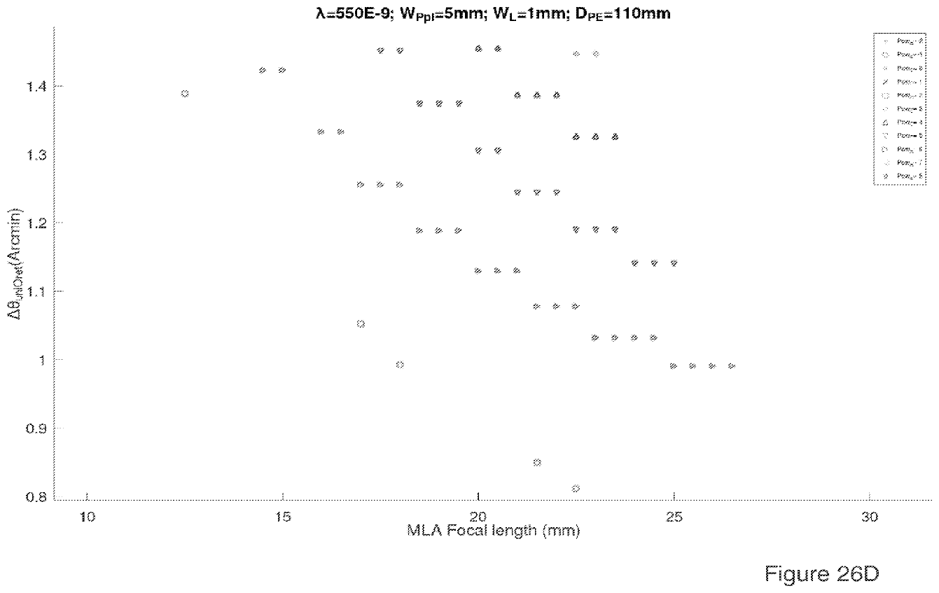

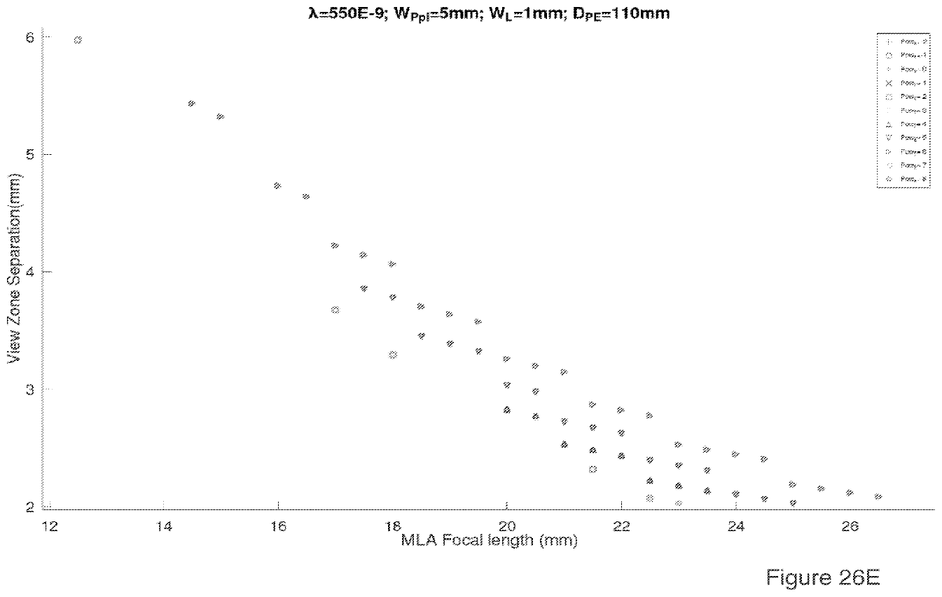

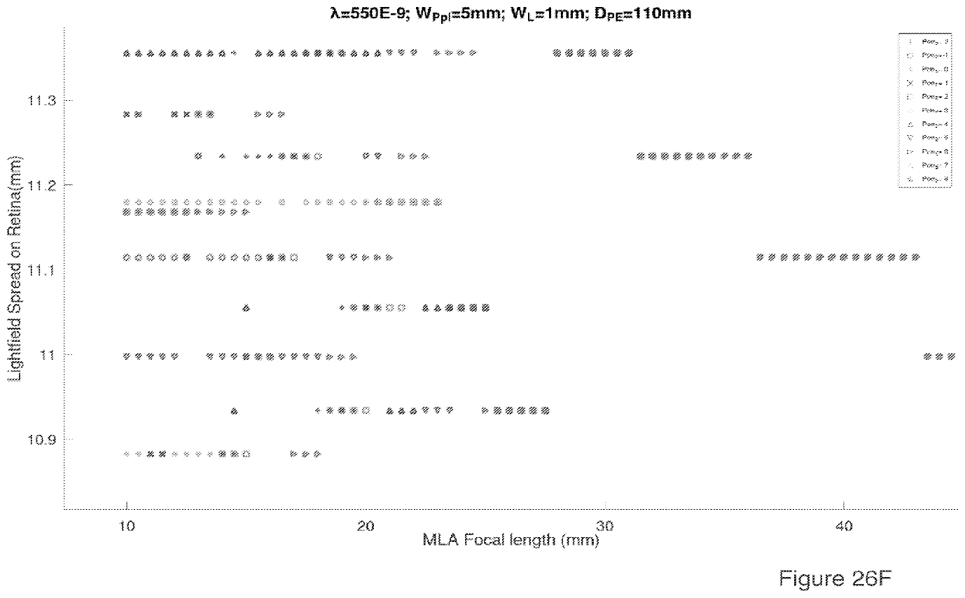

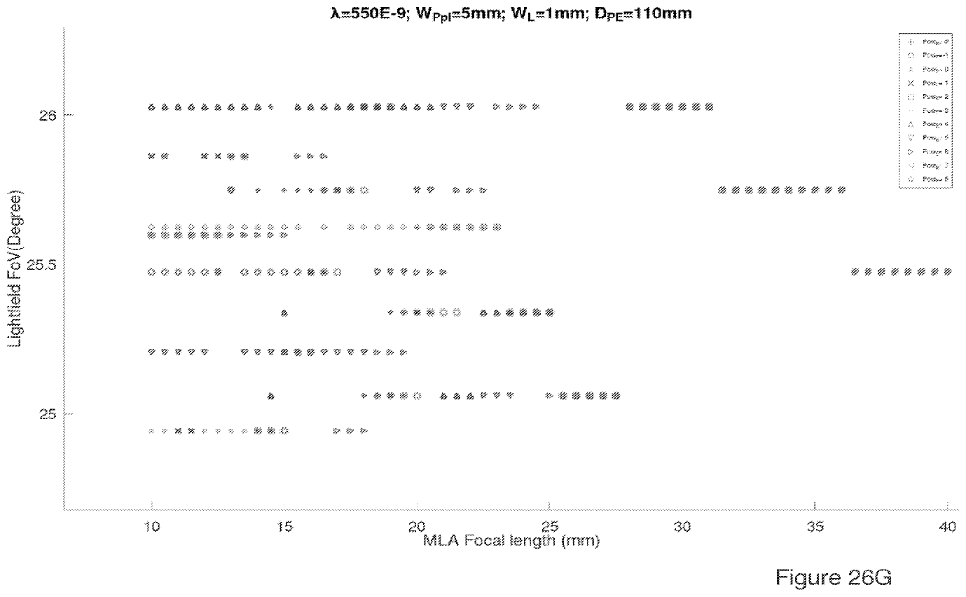

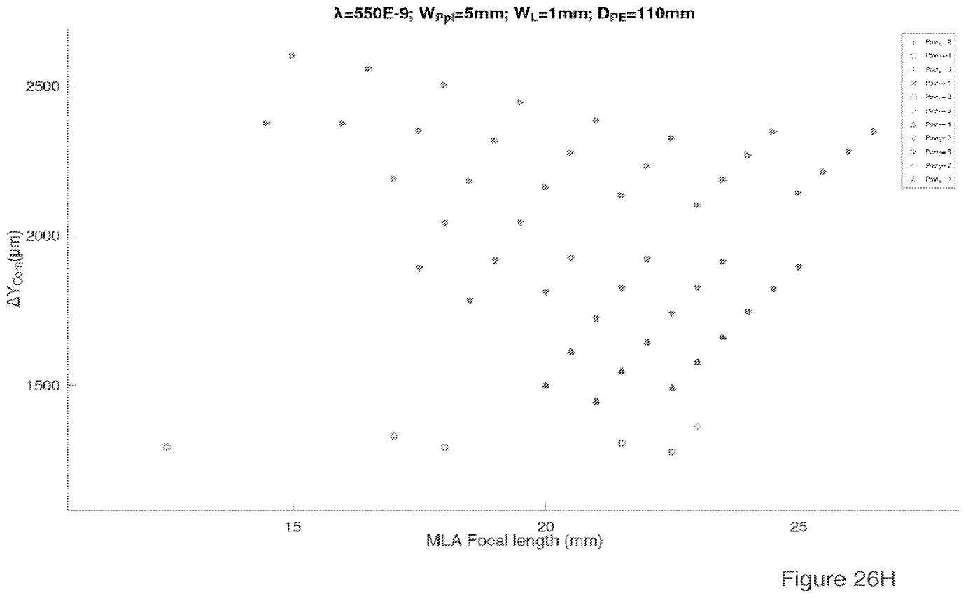

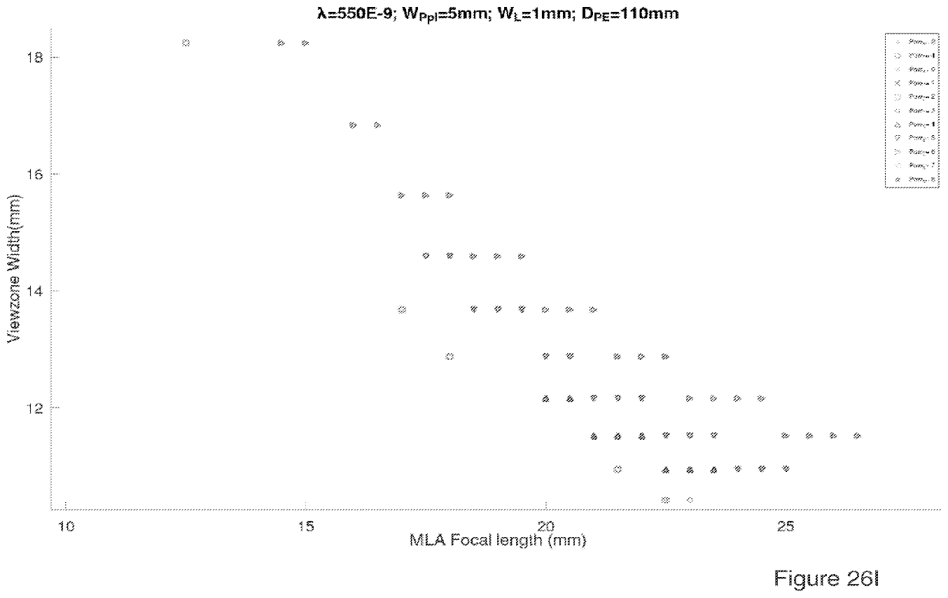

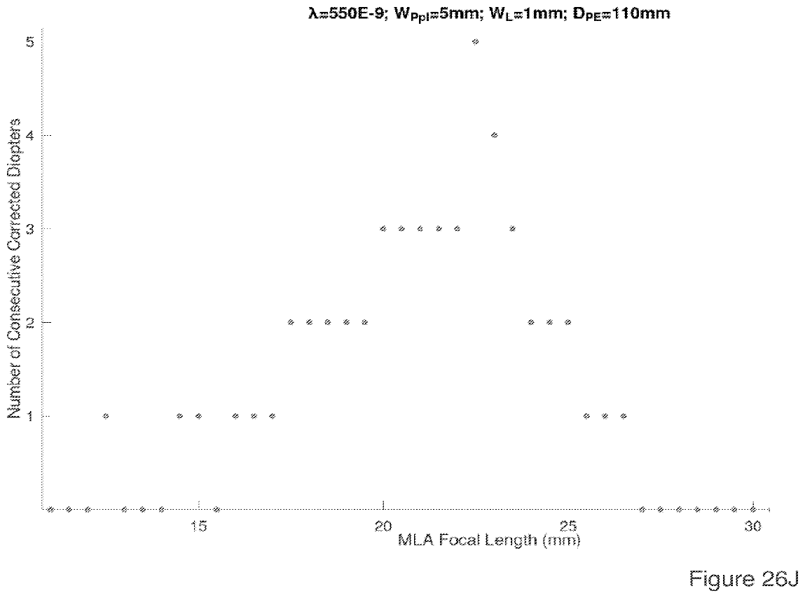

For instance, FIGS. 26A to 26J are exemplary plots of different parameters of one such embodiment. In this example, a display screen comprising a 24 μm pixel pitch is disposed 110 mm from the eye of a user. A translatable LFSL layer comprising an MLA with a 1 mm pitch ˜22.5 mm focal length is also employed. In comparison to the exemplary embodiments described above, a content quality parameter comprising a 1.5 arcminute angular resolution threshold may be applied, which results in a continuous perceptive correction range of 4 diopters, corresponding to perception adjustments of 2 to 6 diopters eye accommodation power. This range may, in accordance with one embodiment, be shifted by placing, for instance, a 2-diopter lens in front of the eye(s) of a user, enabling perception adjustments for eye accommodation power between 0 and 4 diopters, corresponding to an accommodation range of 25 cm to infinity. Furthermore, this may allow for a minimum view zone size of ˜10.4 mm, and a minimum separation of 2.07 mm between a centred pupil of 5 mm diameter and the neighbouring view zone edge. This may allow for the user's eye pupil(s) to move at least 2.07 mm (or 9.5 degrees) to either side of centre without resulting in an overlap with neighboring view zones, or a dark area in which the user cannot perceive visual content. In some embodiments, a pupil position tracking system can be used to update a rendered light field and shift a view zone position along with view zone size control via software to further increase the possible range of motion of the pupil without adversely impacting performance of the device. While such aspects may be less important for acuity assessments, they may be useful in, for instance, saccade or pursuit tests of a cognitive assessment, where a user's eyes are not fixed. Accordingly, employing a translatable LFSL enables various system components, specifications, and/or geometries that may be more optimally suited to a particular application.

In this exemplary embodiment, FIG. 26A is a plot of the MLA-to-display distance as a function of MLA focal length for different dioptric powers. FIG. 26B is a plot of the corresponding focus spot size at the retina. Similarly, FIG. 26C is a plot of the corresponding cut-off angular pitch/resolution values as a function of MLA focal length. FIG. 26D is a plot of the corresponding angular pitch/resolution for a uniform distribution of focused beams on the retina. FIG. 26E is a plot of view zone separation from a 5 mm diameter reference pupil, while FIG. 20F is a plot of spread on the retina. FIG. 26G is a plot of the corresponding field of view as a function of MLA focal length. FIG. 26H is a plot of the beam size on the cornea. FIG. 26I is a plot of the view zone width as a function of MLA focal length. Finally, FIG. 26J is a plot of the maximum number of continuous dioptric powers (separated by steps of 1 diopter) that can be corrected for by changing the MLA-to-display distance.

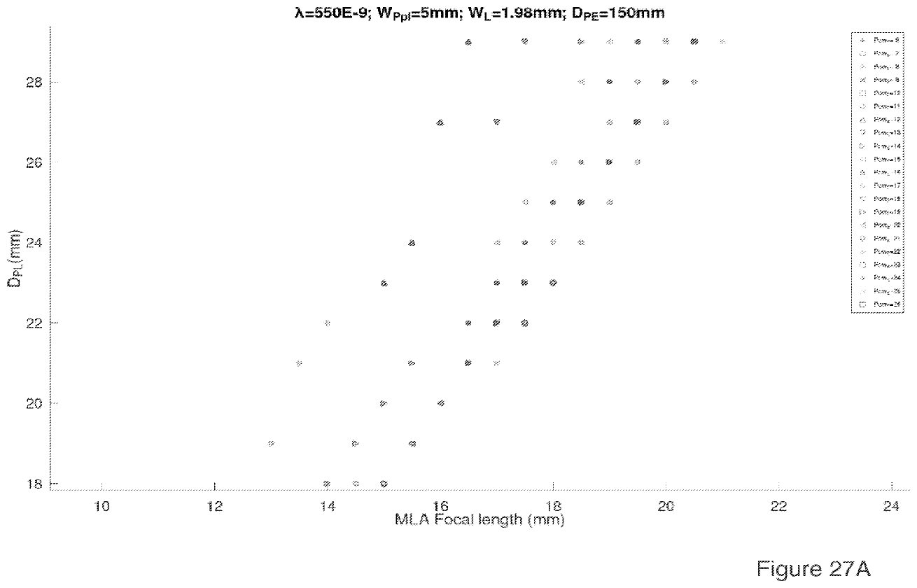

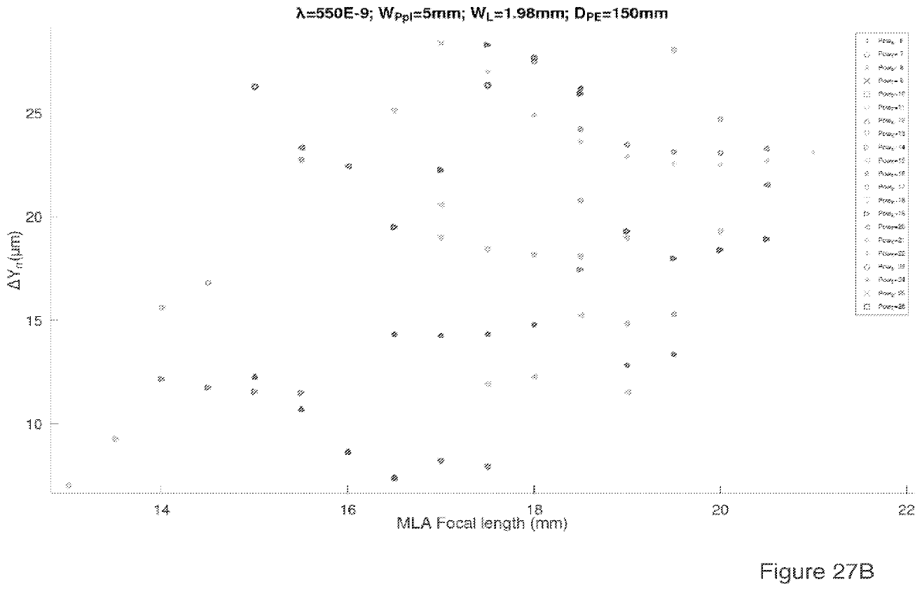

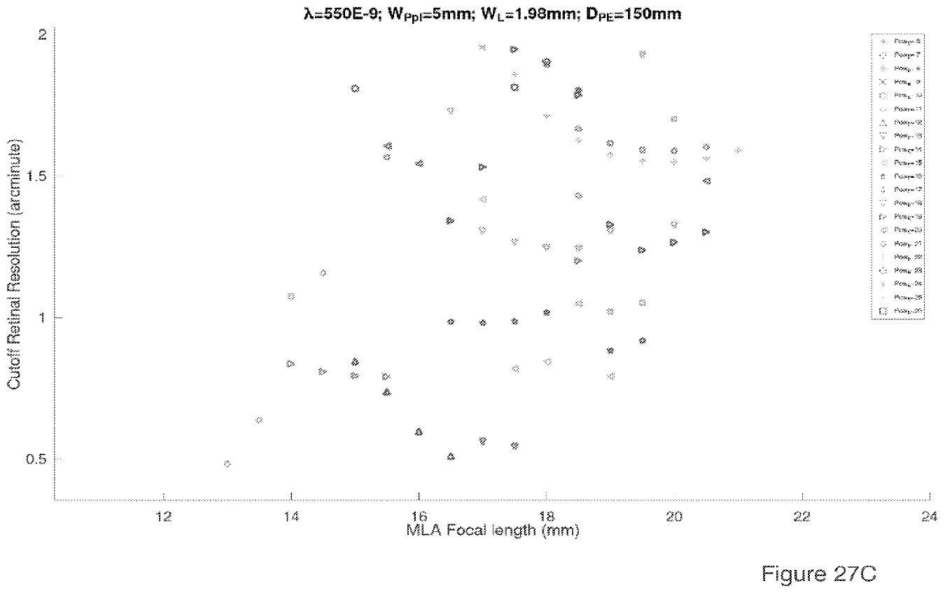

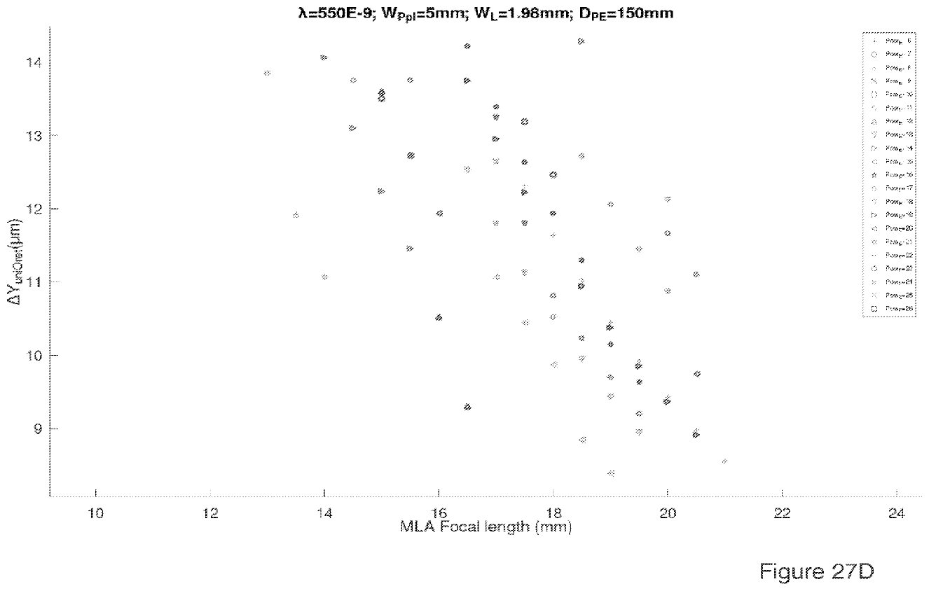

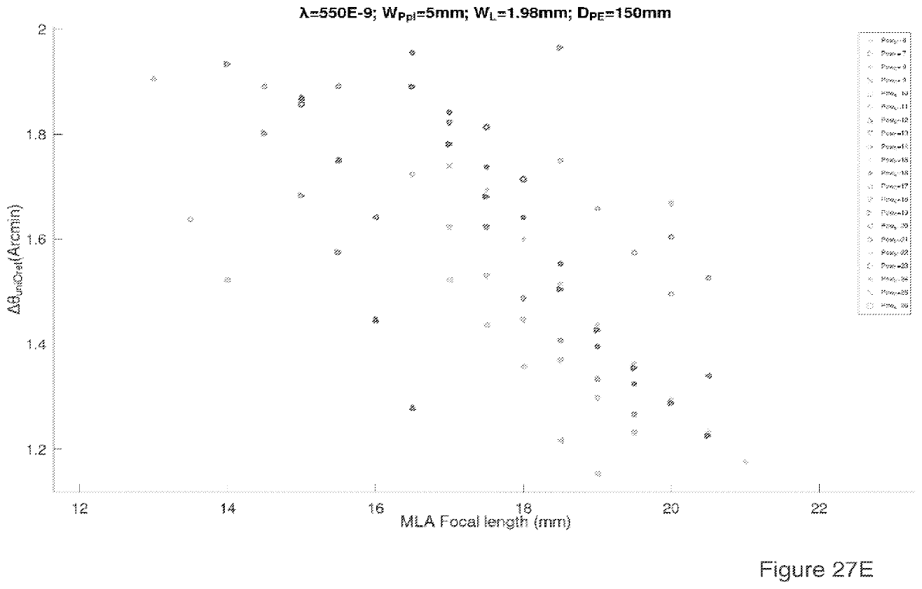

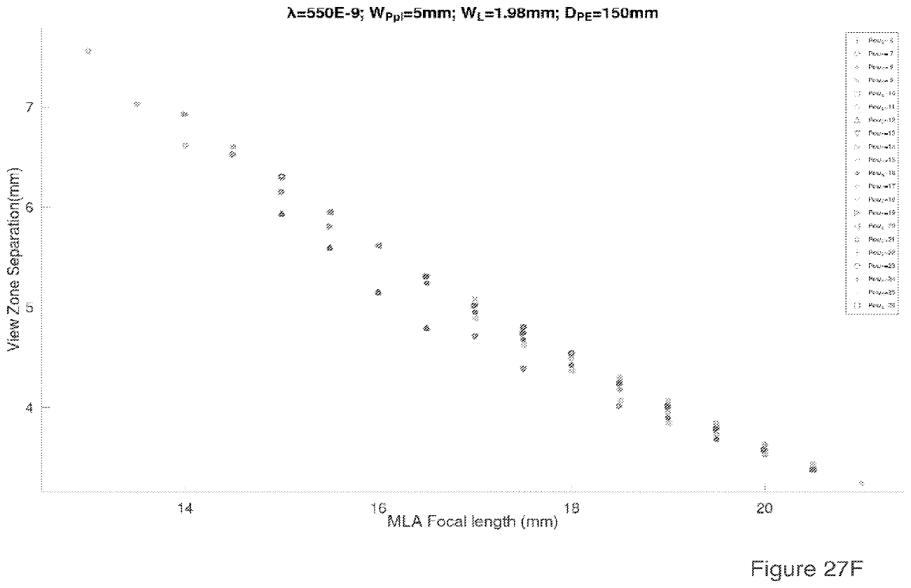

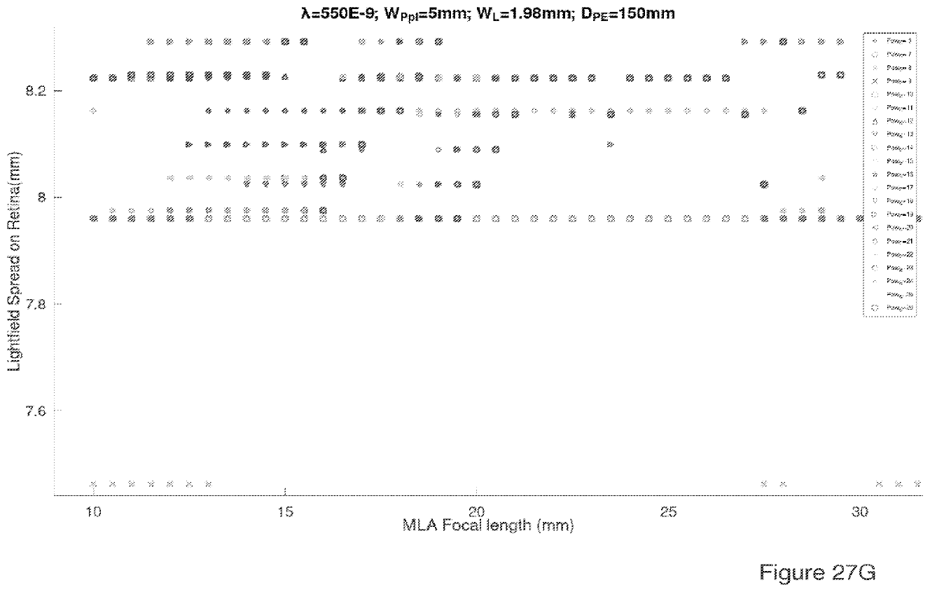

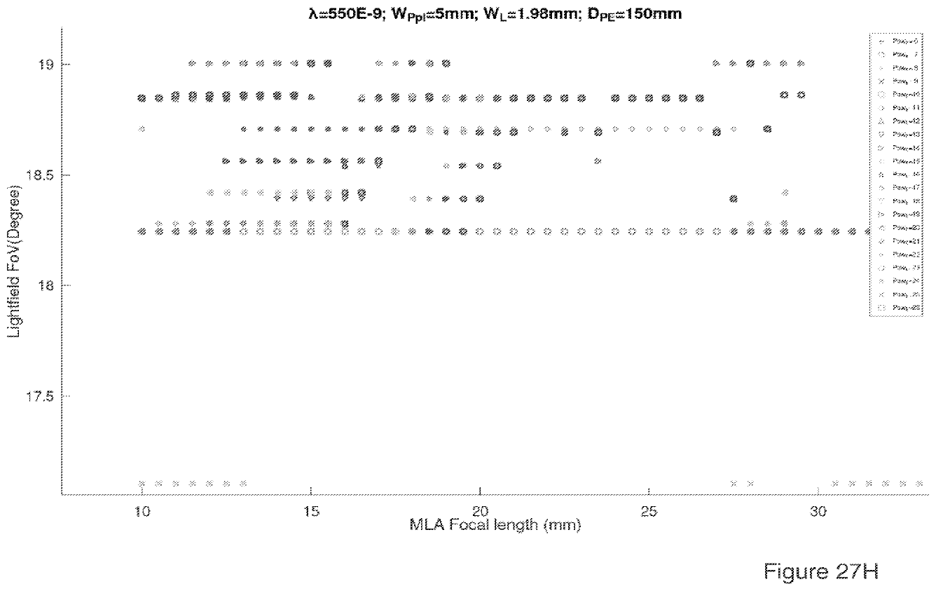

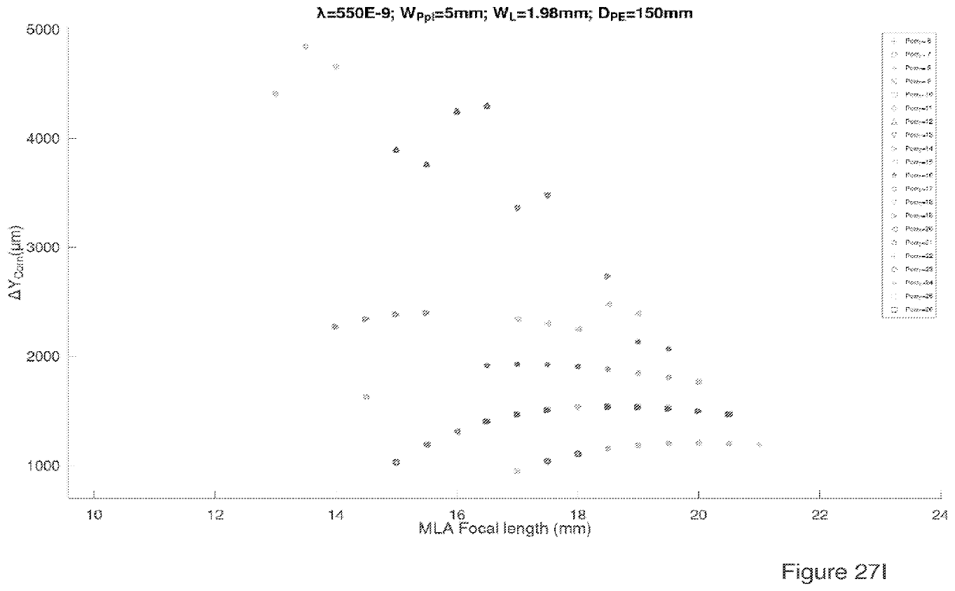

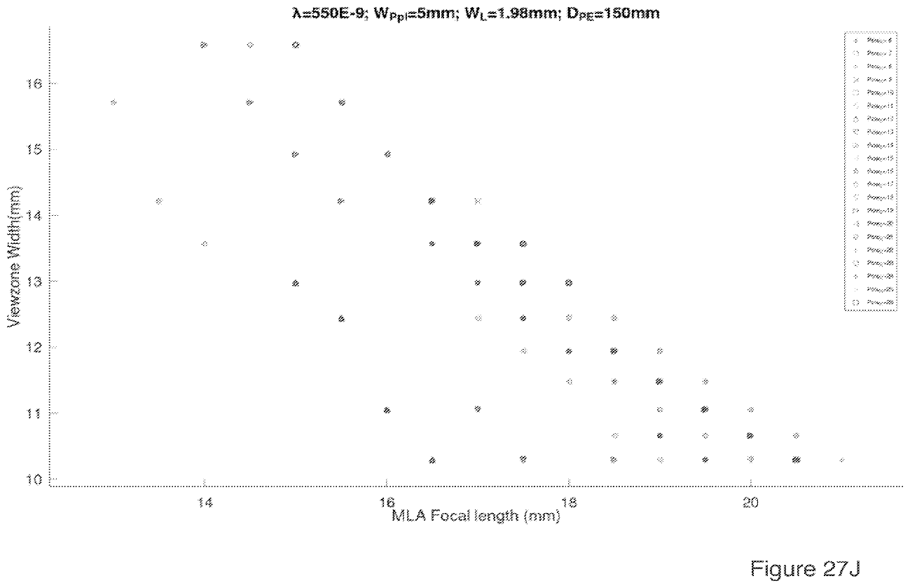

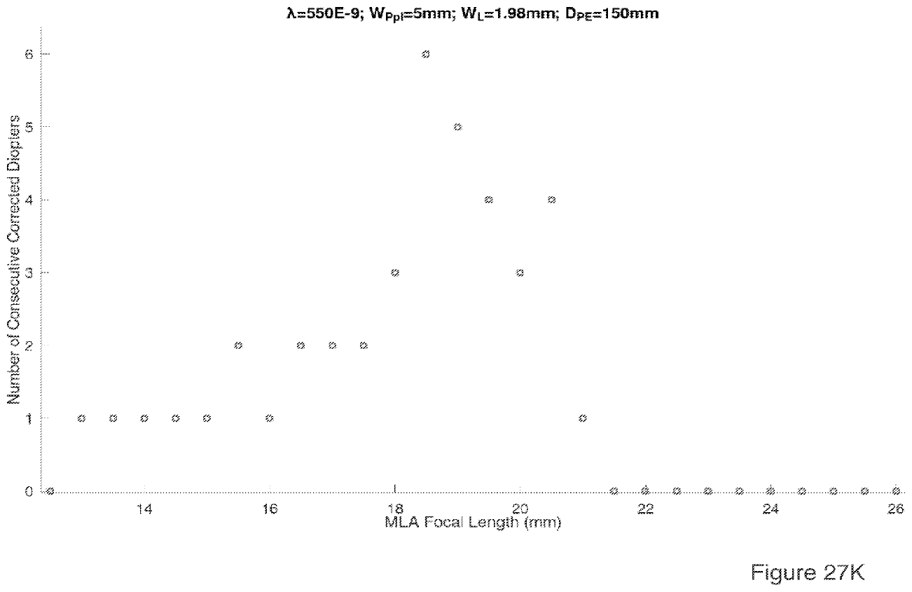

Similarly, FIGS. 27A to 27K are exemplary plots of various parameters corresponding to another light field system. In this example, a 24 μm pixel pitch display was disposed 150 mm in front of the eye, while the LFSL comprised a pitch of 1.98 mm and a focal length of ˜18.5 mm. This configuration allowed for a continuous perception adjustment range of 5 diopters, between 14 and 19 diopters, with a 2-arcminute cut-off angular pitch/resolution. Again, this range may be shifted as needed, for instance by placing a 14-diopter lens in front of the eye, resulting in a light field power correction range for 0 to 5 diopters of accommodation eye power, which corresponds to accommodation range of 20 cm to infinity. Further, this may allow for a minimum view zone size of ˜10 mm, and a minimum of 4 mm separation between a centered pupil of 5 mm diameter and the next view zone edge. This may allow pupil movement of over 2.5 mm to each side without experiencing overlap with neighbouring view zones or a dark region, again benefiting assessments related to, for instance, saccades, pursuit, vergence, or the like. Again, in some embodiments, a pupil position tracking system can be used to update a rendered light field and shift a view zone position along with view zone size control via software to further increase the possible range of motion of the pupil without adversely impacting performance of the device.

In this exemplary embodiment, FIG. 27A is a plot of the MLA-to-display distance as a function of MLA focal length for different dioptric powers. FIG. 27B is a plot of the corresponding focus spot size at the retina. Similarly, FIG. 27C is a plot of the corresponding cut-off angular pitch/resolution values as a function of MLA focal length. FIG. 27D is a plot of the uniform spatial pitch between focused beams on the retina, while FIG. 27E is a plot of the corresponding angular pitch/resolution for a uniform distribution of focused beams on the retina. FIG. 27F is a plot of view zone separation from a reference pupil of 5 mm diameter, while FIG. 27G is a plot of spread on the retina. FIG. 27H is a plot of the corresponding field of view as a function of MLA focal length. FIG. 27I is a plot of the beam size on the cornea, and FIG. 27J is a plot of the view zone width. Finally, FIG. 27K is a plot of the maximum number of continuous dioptric powers that can be corrected for by changing the MLA-to-display distance.

More generally, various embodiments relate to a dynamic LFSL system in which a system of one or more LFSLs may be incorporated on an existing display operable to perception-adjusted content. Such embodiments may, for instance, relate to a clip-on solution that may interface and/or communicate with a display or digital application stored thereon, either directly or via a remote application (e.g. a smart phone application) and in wired or wireless fashion. Such a LFSL may be further operable to rotate in the plane of a display via, for instance, actuators as described above, to improve user experience by, for instance, introducing a pitch mismatch offset between light field shaping elements and an underlying pixel array. Such embodiments therefore relate to a LFSL that is dynamically adjustable/reconfigurable for a wide range of existing display systems (e.g. televisions, dashboard displays in an automobile, a display board in an airport or train terminal, a refractor, a smartphone, or the like).

Some embodiments relate to a standalone light field shaping system in which a display unit comprises a LFSL and smart display (e.g. a smart TV display having a LFSL disposed thereon). Such systems may comprise inherently well calibrated components (e.g. LFSL and display aspect ratios, LFSL elements and orientations appropriate for a particular display pixel or subpixel configuration, etc.).

In either a detachable LFSL device or standalone dynamically adjustable display system, various systems herein described may be further operable to receive as input data related to one or more view zone and/or user locations, or required number thereof (e.g. two or three view zones in which to display perception-adjusted content). For instance, data related to a user location may be entered manually or semi-automatically via, for example, a TV remote or user application (e.g. smart phone application). For example, a television or LFSL may have a digital application stored thereon operable to dynamically adjust one or more LFSLs in one or more dimensions, pitch angles, and/or pitch widths upon receipt of user instruction, via manual clicking by a user of an appropriate button on a TV remote or smartphone application. In accordance with various embodiments, a number a view zones may be similarly selected.

In applications where there is one-way communication (e.g. the system only receives user input, such as in solutions where user privacy is a concern), a user may adjust the system (e.g. the distance between the display and a LFSL, etc.) with a remote or smartphone application until they are satisfied with the display of one or more view zones. Such embodiments may alternatively relate to, for instance, remote eye exams, wherein a doctor remotely adjusts the configuration of a display and LFSL. Such systems may, for instance, provide a high-performance, self-contained, simple system that minimises complications arising from the sensitivity of view zone quality based on, for instance, minute differences from predicted relative component configurations as predicted by, for instance, Equations 1 to 7 above, component alignment, user perception, and the like.

The skilled artisan will appreciate that while a smartphone application or other like system may be used to communicate user preferences or location-related data (e.g. a quality of perceived content from a particular viewing zone), such an application, process, or function may reside in a system or application and be executable by a processing system associated with the display system. Furthermore, data related to a user or viewing location may comprise a user instruction to, for instance, adjust a LFSL, based on, for instance, a user perception of an image quality, or the like.

Alternatively, or additionally, a receiver, such as a smartphone camera and digital application associated therewith, may be used to calibrate a display, in accordance with various embodiments. For instance, a smartphone camera directed towards a display may be operable to receive and/or store signals/content emanating from the LFSL or display system. A digital application associated therewith may be operated to characterise a quality of a particular view zone through analysis of received content and adjust the LFSL to improve the quality of content at the camera's location (e.g. to improve on a calculated LFSL position relative to display that was determined theoretically, for instance using one or more of Equations 1 to 7 above).

For instance, a calibration may be initially performed wherein a user positions themselves in a desired viewing location and points a receiver at a display generating red and blue content for respective first and second view zones. A digital application associated with the smartphone or remote receiver in the first viewing location may estimate a distance from the display by any means known in the art (e.g. a subroutine of a smartphone application associated with a light field display and operable to measure distances using a smartphone sensor). The application may further record, store, and/or analyse (e.g. in mobile RAM) the light emanating from the display to determine whether or not, and/or in which dimensions, angle, etc., to adjust a dynamic LFSL to maximise the amount of red light received in the first view zone while minimising that of blue (i.e. reduce cross talk between view zones).

For example, and in accordance with some embodiments, a semi-automatic LFSL may self-adjust until a digital application associated with a particular view zone receives less than a threshold value of content from a neighbouring view zone (e.g. receives at least 95% red light and less than 5% blue light, in the abovementioned example). The skilled artisan will appreciate that various algorithms and/or subroutines may be employed to this end. For instance, a digital application subroutine may calculate an extent of crosstalk occurring between view zones, or a degree of image sharpness corresponding to an intended perception adjustment (e.g. −6.0 dioptric error correction), to determine in which ways displayed content is blended or improperly adjusted based on content received, to determine which LFSL parameters may be optimised to actuate an appropriate system response. Furthermore, the skilled artisan will appreciate that various means known in the art for encoding, displaying, and/or identifying distinct content may be applied in such embodiments. For example, a display having a LFSL disposed thereon may generate distinct content corresponding to a perception adjustment or dioptric shift that may comprise one or more of, but is not limited to, distinct colours, IR signals, patterns, or the like, to determine a displayed content quality, and initiate compensatory adjustments in a LFSL.

Furthermore, and in accordance with yet further embodiments, a semi-automatic LFSL calibration process may comprise a user moving a receiver within a designated range or region (e.g. a user may move a smartphone from left to right, or forwards/backwards) to acquire display content data. Such data acquisition may, for instance, aid in LFSL layer adjustment, or in determining a LFSL configuration that is acceptable for one or more users of the system within an acceptable tolerance (e.g. all users receive 95% of their intended display content, or a resolution of at least 1 arcsecond is achieved in an eye examination device) within the geometrical limitations of the LFSL and/or display.

The skilled artisan will appreciate that user instructions to any or all of these ends may be presented to a user on the display or smartphone/remote used in calibration for ease of use (i.e. guide the user in during calibration and/or use). Similarly, if, for instance, physical constraints (e.g. LSFL or display geometries) preclude an acceptable adjusted image resolution, an application associated with the display, having performed the appropriate calculations, may guide a user to move to a different location (or to move the display) to provide for a better experience.

In yet other embodiments, one or more user locations may be determined automatically by a display system. For instance, a pupil location may be determined via the use of one or more cameras or other like sensors and/or means known in the art for determining user, head, and/or eye locations, and dynamically adjusting a LFSL in one or more dimensions to render content so to be displayed at one or more appropriate locations. Yet other embodiments relate to a self-localisation method and system that maintains user privacy with minimal user input or action required to determine one or more view zone locations, and dynamically adjust a LFSL to display appropriate content thereto.

Yet further applications may utilise a dynamic light field shaping layer subjected to oscillations or vibrations in one or more dimensions in order to, for instance, improve perception of an image generated by a pixelated display. Or, such a system may by employed to increase an effective view zone size so as to accommodate user movement during viewing. For example, a LSFL may be vibrated in a direction perpendicular to a screen so to increase a depth of a view zone in that dimension to improve user experience by allowing movement of a user's head towards/away from a screen without introducing a high degree of perceived crosstalk, or to improve a perceived image brightness.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

### LIST OF TERMINOLOGY

D_{E}: Eye depth
D_{PL}: Horizontal distance between the pixel (display) and the lenslet (multi-lens array)
D_{LE}: Horizontal distance between the lenslet and the Eye.
δθ_{ZC}^{Rect}: divergance angle of rectangular aperature baser on diffracted sinc 1st zero crosing.
Δθ_{Req}^{p}: Pixel(display) angular view required.
Δᵣₑ; Δθᵣᵣ: beam divergence after the eye lens, eam divergence on retina.
Δᵣₑ^{Res}: Angular separation between the rays from adjacent pixels on the eye lens.
Δθᵣₑ^{ab}: Beams a and b angle difference after the eye lense.
Δθ_{Mre}^{ab}: Difference in marginal rays a and b (divergence) after the eye lens.
Δyᵣₑ^{ab}: Difference in positions of rays a and b on eye lens.
Δyᵣₑ^{Res}: The separation between the rays from adjacent pixels on the eye lens.
Δy_{Mre}^{ab}: Difference in positions of marginal rays a and b on eye lens.
Δyᵣₑₜ: Retinal spot spacing per one nodal band.
Δy_{dis}^{ΔN}: Nodal band width on the display.
Δyₚ: Pixel pitch.
Δyᵣₑₜ^{req}: Required retinal spot spacing.
Δyᵣₑ; Δyᵣᵣ: bean size on the eye lens; retinal beam spot size (based on ray tracing).
Δyᵣᵣ^{ΔN}: Retinal spot spacing in one nodal ray band.
Δyᵣᵣ^{N}: Retinal Nodal spots(band centers)spacing.
Δyₒᵣₑₜ^{uni}: Uniform retinal spots spacing resulting from overlapping nodal bands on retina.
Δyᵣₑ^{ab}: difference in rays a and b positions on eye.
f_{E}: Eye focal length.
f_{L}: MLA lenslet focal length.
FoV: Field of View
λ: wavelength.
MLA: Multi-lens array.
N_{P}^{ΔN}: Number of pixels in one nodal band.
Oᵣₑₜ: Number of overlapping nodal bands on retina.
P_{L}: Pitch of MLA lenslets.
θᵣₗ: ray angle after the MLA lenslet.
θᵣₚ: Pixel ray angle.
θᵣₑ: ray angle after the eye lens.
Sprdᵣₑₜ: Light field spread width on retina.
W_{Disp}: display width (size).
W_{L}: Lenslet width.
Wₚₚₗ: Pupil width (diameter).
W_{Mrr}: diffraction based beam size on retina.
y_{L}; y_{L}^{a}, y_{L}^{b}: MLA lenslet position, positions of lenslets a and b.
y_{P}, yₚ^{a}, y_{P}^{b}: Pixel position, pixel a position, pixel b position.
yᵣₑ: ray position on the eye lens.
yᵣₗ; yᵣₗ^{b}; ray position on the MLA lenslet; positions of rays a and b on NIA lenslets.

1. A light field shaping system for interfacing with light emanated from pixels of a digital display to govern display of perception-adjusted content, the system comprising:
a light field shaping layer (LFSL) comprising an array of light field shaping elements and disposable relative to the digital display so to align said array of light field shaping elements with the pixels in accordance with a current light field shaping geometry to thereby define a perception adjustment of displayed content in accordance with said current geometry;
an actuator operable to adjust an optical path length between said LFSL and the digital display to adjust alignment of said light field shaping elements with the pixels in accordance with an adjusted geometry thereby defining an adjusted perception adjustment of displayed content in accordance with said adjusted geometry; and
a digital data processor operable to activate said actuator to translate said LFSL to adjust the perception-adjusted content.

2. The light field shaping system of claim 1, wherein said LFSL comprises a microlens array.

3. The light field shaping system of claim 1, wherein said adjusted perception adjustment corresponds to a reduced visual acuity of a user.

4. The light field shaping system of claim 1, wherein said actuator is operable to translate said LFSL in a direction perpendicular to the digital display.

5. The light field shaping system of claim 1, wherein said light field shaping geometry relates to a physical distance between the digital display and said LFSL.

6. The light field shaping system of claim 1, wherein said adjusted geometry corresponds to a selectable range of perception adjustments of displayed content, wherein distinctly selectable geometries correspond with distinct selectable ranges of perception adjustments.

7. The light field shaping system of claim 6, wherein said distinct selectable ranges comprise distinct dioptric correction ranges.

8. The light field shaping system of claim 1, wherein said digital data processor is further operable to:
receive as input a requested perception adjustment as said adjusted perception adjustment;
based at least in part on said requested perception adjustment, calculate an optimal optical path length to thereby define an optimal geometry as said adjusted geometry; and
activate said actuator to adjust said optical path length to said optimal optical path length and thereby optimally achieve said requested perception adjustment.

9. The light field shaping system of claim 1, wherein said digital data processor is further operable to:
receive as input feedback data related to a quality of said adjusted perception adjustment; and
dynamically adjust said optical path length via said actuator in response to said feedback data.

10. The light field shaping system of claim 1, wherein the light field shaping system comprises a system for administering a vision-based test.

11. The light field shaping system of claim 10, wherein said vision-based test comprises a visual acuity examination, and the perception-adjusted content comprises an optotype.

12. The light field shaping system of claim 10, wherein said vision-based test comprises a cognitive impairment test.

13. The light field shaping system of claim 1, wherein said actuator selectively introduces an optical path length-increasing medium within said optical path length to selectively adjust said optical path length.

14. The light field shaping system of claim 1, wherein said digital data processor is operable to translate said LFSL to adjust the perception-adjusted content while satisfying a visual content quality parameter.

15. The light field shaping system of claim 14, wherein said visual content quality parameter comprises one or more of a perception-adjusted content resolution, a corneal beam size, a view zone size, or a distance between a pupil and a view zone edge.

16. A method for dynamically adjusting a perception adjustment of displayed content in a light field display system comprising a digital processor and a digital display defined by an array of pixels and a light field shaping layer (LFSL) disposed relative thereto, the method comprising, by said digital processor:
accessing display geometry data related to one or more of the light field display system and a user thereof, said display geometry data at least in part defining the perception adjustment of displayed content;
digitally identifying a preferred display geometry based, at least in part, on said display geometry data, said preferred display geometry comprising a desirable optical path length between said LFSL and the pixels to optimally produce a requested perception adjustment of displayed content; and
automatically adjusting said optical path length, via the digital processor and an actuator operable to adjust said optical path length and thereby adjust the perception adjustment of displayed content to said requested perception adjustment.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. The method of claim 16, wherein the light field display system comprises a system for administering a vision-based visual acuity examination, and the perception-adjusted content comprises an optotype.

33. The method of claim 16, wherein the light field display system comprises a system for administering a cognitive impairment test.

34. The method of claim 16, further comprising:
receiving as input a requested perception adjustment as said adjusted perception adjustment;
based at least in part on said requested perception adjustment, calculating an optimal optical path length to thereby define an optimal geometry as said adjusted geometry; and
activating said actuator to adjust said optical path length to said optimal optical path length and thereby optimally achieve said requested perception adjustment.

35. The method of claim 16, further comprising:
receiving as input feedback data related to a quality of said adjusted perception adjustment; and
dynamically adjusting said optical path length via said actuator in response to said feedback data.

36. The method of claim 16, wherein said preferred display geometry corresponds to a selectable range of perception adjustments of displayed content, wherein distinctly selectable geometries correspond with distinct selectable ranges of perception adjustments, wherein said distinct selectable ranges comprise distinct dioptric correction ranges.
